(19) United States
(12) Patent Application Publication
(10) Pub. No.: US 2024/0263251 A1
(43) Pub. Date: Aug. 8, 2024

(54) COMPOSITIONS AND METHODS FOR PROVIDING SECONDARY BILE ACIDS TO A SUBJECT

(71) Applicant: Finch Therapeutics Holdings LLC, Boston, MA (US)

(72) Inventors: Mark SMITH, Cambridge, MA (US); Sonia TIMBERLAKE, Brookline, MA (US); Rotem Gura SADOVSKY, San Antonio, TX (US); Daniel PETERSON, Seattle, WA (US); Christopher WEIDENMAIER, Brookline, MA (US)

(73) Assignee: Finch Therapeutics Holdings LLC, Boston, MA (US)

(21) Appl. No.: 18/451,985

(22) PCT Filed: Feb. 18, 2022
(86) PCT No.: PCT/US2022/017049

§ 371 Date: Aug. 18, 2023

Related U.S. Application Data
(60) Provisional application No. 63/218,122, filed on Jul. 2, 2021.
(60) Provisional application No. 63/163,497, filed on Mar. 19, 2021.
(60) Provisional application No. 63/151,495, filed on Feb. 19, 2021.

(51) Int. Cl. C12Q 1/689; A61K 35/74; A61K 38/44; A61K 48/00; A61P 3/00

(52) U.S. Cl. CPC C12Q 1/689; A61K 35/74; A61K 38/44; A61K 48/005; A61P 3/00; C12Y 101/01145; C12Y 103/01003; C12Y 103/01022; C12Q 2600/106

(57) ABSTRACT

The present disclosure relates to microbial compositions for enhancing bile acid metabolism in a subject, methods of manufacturing the compositions, and methods of selecting subjects in need of treatment with the compositions. Certain bile acid transforming or converting genes and bile acid transforming or converting bacteria are used to select desired bacterial isolates or suitable donors or patients, predict treatment success, or track treatment response.

### CROSS-REFERENCE TO RELATED APPLICATIONS

This application claims the benefit of U.S. Provisional Application No. 63/151,495, filed Feb. 19, 2021, U.S. Provisional Application No. 63/163,497, filed Mar. 19, 2021, and U.S. Provisional Application No. 63/218,122, filed Jul. 2, 2021, all of which are incorporated by reference in their entireties herein.

### INCORPORATION-BY-REFERENCE OF MATERIALS FILED ON A COMPACT DISC

The instant application contains a Sequence Listing which has been submitted electronically in ASCII format and is hereby incorporated by reference in its entirety. Said ASCII copy, created on Feb. 18, 2022, is named P35054WO00_SL.txt and is 931,093 bytes in size.

### BACKGROUND

Implantation or administration of human colonic microbiota into the bowel of a sick patient is called Fecal Microbiota Transplantation (FMT), also commonly known as fecal bacteriotherapy. FMT is believed to repopulate the gut with a diverse array of microbes that control key pathogens by creating an ecological environment inimical to their proliferation and survival. It represents a therapeutic protocol that allows a fast reconstitution of a normal compositional and functional gut microbial community.

FMT has been used to treat Clostridium difficile infection (CDI). FMT has also been suggested in treating other gut infective agents such as E. coli and Vancomycin resistant Enterococci (VRE). It entails infusions through a colonoscope, an enema or via a nasojejunal tube of human microbiota either in the form of homogenized stool, or cultured stool components such as Clostridia, to implant in the colon and thereby displace or eradicate pathogenic bacteria, e.g., C. difficile. Fecal bacteriotherapy has also been successful in treating conditions having a neurological component, such as ASD, Parkinson's Disease, and Multiple Sclerosis and Chronic Fatigue Syndrome.

Compositions for performing FMT typically incorporate the fecal microbiota of stool collected from healthy human donors. However, the microbial content of stool is not uniform across donors, or even longitudinally across different samples collected from the same donor. As a result, different doses of an FMT composition can vary in microbial constitution, for example in the presence or absence of a specific species or strain of bacteria or in the relative abundance of a bacterial species or strain.

An inability to regulate the identity and/or relative abundances of species or strain of bacteria in an FMT formulation may result in reduced and/or variable efficacy when administered to patients, if a particular bacterial species or strain or group of species or strain is associated with or needed for the treatment of a condition, for example by inducing a therapeutic response in a patient's cells. Further, a patient population to be administered a live biotherapeutic product may vary significantly in the identities and relative abundances of bacterial species or strain in the microbiome, even among patients exhibiting similar symptoms.

### SUMMARY

In an aspect, the present disclosure provides a method of selecting a subject in need of supplementation with bile acid transforming bacteria that produce a secondary bile acid from a primary bile acid, the method comprising: determining a number of bacterial clades or species in a microbiome of the individual that comprise a nucleic acid comprising a bile acid metabolism consensus sequence associated with production of the secondary bile acid from the primary bile acid; and selecting the individual as the subject on the basis of determining that the number of bacterial clades or species is less than a threshold value.

In another aspect, the present disclosure provides a method of treating a deficiency in the metabolism of a primary bile acid to a secondary bile acid in a subject in need thereof, the method comprising: administering a bacterial isolate to the subject to treat the deficiency, wherein the bacterial isolate comprises a bai gene.

In another aspect, the present disclosure provides a method of manufacturing a pharmaceutical composition for administration to a subject to alleviate a deficiency in bile acid metabolism, the method comprising: selecting a bacterial isolate from a stool of a human donor on the basis of a sequence identity of a nucleic acid of the bacterial isolate to a nucleic acid of a bacterial clade or species in a fecal microbiota of a subject who does not have a history of or is in remission from a disorder related to an intestinal dysbiosis, wherein the nucleic acid comprises a bile acid metabolism consensus sequence that codes for all or a portion of an enzyme that functions in a metabolic pathway producing a primary or a secondary bile acid, and wherein the sequence identity is at least 97%; and incorporating the bacterial isolate into the pharmaceutical composition.

In another aspect, the present disclosure provides a method, comprising: selecting a stool of a donor on the basis of the presence of one or more strains of bile acid metabolizing bacteria in the donor, wherein the one or more strains of bile acid metabolizing bacteria are capable of producing a bile acid by transforming a primary bile acid to a secondary bile acid or a first secondary bile acid to a second secondary bile acid; isolating the one or more strains of bile acid metabolizing bacteria; and incorporating the bile acid metabolizing bacteria into a pharmaceutical composition.

In a further aspect, the present disclosure provides a method of treating inflammatory bowel disease in a subject in need thereof, the method comprising: determining that a fecal microbiota of the subject comprises one or fewer bacterial clades or species capable of transforming a primary bile acid to a secondary bile acid; and administering to the subject a bacterial species or strain comprising one or more bai genes.

In yet another aspect, the present disclosure provides a method of treating a secondary bile acid deficiency in a subject lacking a functional bai operon, the method comprising administering to the subject a bacterial species or strain comprising the functional bai operon.

In another aspect, the present disclosure provides a method of restoring a bai operon to a microbiome of a subject lacking a functional copy of the bai gene operon, the method comprising administering to the subject a bacterial isolate comprising the bai gene operon.

In a further aspect, the present disclosure provides a method of treating a disorder related to an intestinal dysbiosis in a subject in need thereof, the method comprising: determining that a fecal microbiota of the subject comprises three or fewer bacterial clades or species comprising a bile acid metabolism consensus sequence, wherein a bacterial clade or species comprising the bile acid metabolism consensus sequence produces a gene product that catalyzes a step in a metabolic pathway that converts a first secondary bile acid to a second secondary bile acid; and administering to the subject one or more bacterial strains comprising the gene encoding the gene product.

In another aspect, the present disclosure provides a method of treating a subject in need of supplementation with bile acid converting bacteria, the method comprising: determining a number of bacterial clades or species in a microbiome of an individual that comprise a nucleic acid comprising a bile acid metabolism consensus sequence; selecting the individual as the subject on the basis of determining that the number of bacterial clades or species is less than a threshold value; and administering to the subject the bile acid converting bacteria; wherein the bile acid converting bacteria comprises bacteria that produce isoalloLCA.

In another aspect, the present disclosure provides a method of treating a subject in need of supplementation with bile acid converting bacteria, the method comprising: determining a deficiency of a secondary bile acid in the subject, wherein the deficient secondary bile acid comprises at least isoalloLCA; and administering to the subject bile acid converting bacteria, wherein following administration the bile acid converting bacteria produce the isoalloLCA in the subject.

In another aspect, the present disclosure provides a method of manufacturing a pharmaceutical composition for administration to a subject to alleviate a deficiency in bile acid metabolism, the method comprising: selecting a donor based on a presence or abundance of isoalloLCA above a threshold level in a stool of the donor; obtaining at least one bacterial isolate from the stool of the donor, wherein the bacterial isolate comprises a DNA sequence coding for an enzyme that synthesizes isoalloLCA; and incorporating the at least one bacterial isolate into the pharmaceutical composition.

In another aspect, the present disclosure provides a method comprising: selecting a stool on the basis of the presence of one or more bile acid metabolizing bacteria in a stool of a donor, wherein the one or more bile acid metabolizing bacteria are capable of producing isoalloLCA; isolating the one or more bile acid metabolizing bacteria; and incorporating the bile acid metabolizing bacteria into a pharmaceutical composition.

In another aspect, the present disclosure provides a method of treating dysbiosis in a subject in need thereof, the method comprising: determining that a fecal microbiota of the subject comprises one or fewer bacterial clades or species capable of producing isoalloLCA; and administering to the subject at least one bacterial species comprising a gene selected from the group consisting of a gene encoding 5β-reductase, a gene encoding 5α-reductase, a gene encoding 3β-HSDH, and a combination thereof.

In another aspect, the present disclosure provides a method of supplementing a microbiome of a subject with isoalloLCA, the method comprising administering to the subject a DNA sequence that encodes an enzyme that functions in the metabolic pathway that synthesizes isoalloLCA.

### BRIEF DESCRIPTION OF THE DRAWINGS

FIG. 1A: A diagram showing the secondary bile acid levels of Crohn's disease (CD), and ulcerative colitis (UC) subjects relative to subjects that do not have Crohn's or UC.

FIG. 1B: A diagram showing the relationship between the ratio of the secondary bile acid DCA to its corresponding primary bile acid CA (DCA:CA_log 10) and the ratio of the secondary bile acid LCA to its corresponding primary bile acid CDCA (LCA:CDCA_log 10) in non-IBD subjects and patients with UC and CD.

FIG. 2: A diagram showing the correlation between secondary bile acid expression and bai gene abundance.

FIG. 3: A diagram showing the repletion of primary bile acid transforming bacteria predicts a subject's response to fecal microbiota transplantation (FMT).

FIG. 4A: A schematic of the eight bile-acid inducible operon (bai) genes required for primary-to-secondary bile acid transformation.

FIG. 4B: A diagram showing the number of bai genes detected in a patient in the weeks following a single FMT treatment.

FIG. 5A: A diagram showing that a single clade of primary bile acid transforming bacteria in a subject can normalize the secondary bile acid deoxycholate.

FIG. 5B: A diagram showing that a single clade of primary bile acid transforming bacteria in a subject can normalize the secondary bile acid lithocholic acid.

FIG. 6: A diagram showing bile acid transforming bacteria clades in the Firmicutes phylum (left panel) and varying prevalence of bile acid transforming clades between stool from healthy subjects and subjects with UC or CD (right panel).

FIG. 7: A diagram showing the transformation of primary bile acids to secondary bile-acids and the further metabolism of secondary bile acids.

FIG. 8: A diagram showing the relationship between the ratio of secondary bile acid (DCA) to primary bile acid CA (DCA:CA) and the abundance of bile acid transforming bacteria in non-IBD subjects and patients with Crohn's disease (CD).

FIG. 9: A diagram showing responding and non-responding (true or false) Crohn's disease patients' relative abundance shift in bacteria carrying the bai gene operon (y-axis) following administration of FMT in various studies (x-axis). Patients' bile acid transforming bacteria can serve as a prognosis biomarker for FMT-based CD treatments.

FIG. 10A: A diagram showing the luminescence readouts (y-axis) of a cell assay testing the ability of different stimulants comprising different bile acid concentrations (x-axis) to activate TGR5. PBA=primary bile acid; SBA=secondary bile acid; isoSBA1=3-oxo-lithocholic acid (3-oxo-LCA, 30LCA); isoSBA2=isolithocholic acid (isoLCA, ILCA); SBA 1=lithocholic acid (LCA); SBA2=deoxycholic acid (DCA); PBA1=chenodeoxycholic acid (CDCA); PBA2=cholic acid (CA); isoSBA3=alloisolithocholic acid (alloisoLCA, AILCA, isoalloLCA, IALCA); SBA3=ursodeoxycholic acid (UDCA); Pos. control=3-(2-chlorophenyl)-N-(4-chlorophenyl)-N,5-dimethylisoxazole-4-carboxamide.

FIG. 10B: A diagram showing the luminescence readouts (y-axis) of a cell assay testing the ability of different bacterial strains (x-axis) to active TGR5 by testing bacterial strain supernatants after incubation with PBA. PBA=primary bile acid; 1=Clostridium scindens (strain 1); 2=Clostridium scindens (strain 2); 3=Clostridium hylemonae; control=Bacteroides fragilis); PBA1=cholic acid (CA); PBA2=chenodeoxycholic acid (CDCA).

FIG. 11: A diagram showing the abundance of isoalloLCA-producing genes (y-asis) in non-inflammatory bowel disease (non-IBD) samples, Crohn's disease (CD) samples, and ulcerative colitis (UC) samples classified as dysbiotic or normal (x-axis). CPM=counts per million.

### DETAILED DESCRIPTION

Unless defined otherwise, all technical and scientific terms used herein have the same meaning as commonly understood by one of ordinary skill in the art to which this disclosure belongs.

All publications, patents, and patent applications mentioned in this specification are herein incorporated by reference to the same extent as if each individual publication, patent, or patent application was specifically and individually indicated to be incorporated by reference.

As used herein and in the appended claims, the singular forms “a,” “an,” and “the” include plural referents unless the context clearly dictates otherwise. By way of example, “an element” means at least one element and can include more than one element.

As used herein, the term “substantially”, when used to modify a quality, generally allows certain degree of variation without that quality being lost. For example, in certain aspects such degree of variation can be less than 0.1%, about 0.1%, about 0.2%, about 0.3%, about 0.4%, about 0.5%, about 0.6%, about 0.7%, about 0.8%, about 0.9%, about 1%, between 1-2%, between 2-3%, between 3-4%, between 4-5%, or greater than 5% or 10%.

Where a range of values is provided, it is understood that each intervening value, between the upper and lower limit of that range and any other stated or intervening value in that stated range is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either both of those included limits are also included in the disclosure.

As used herein, the term “relative abundance” of a bacterial taxon (e.g., strain or species) refers to relative representation of cells of that taxon among cells of all taxa in the community (e.g., a fecal microbiota, a processed consortium of fecal bacteria or a bacterial mixture). Relative abundance refers to the abundance of cells of a particular species of bacteria by the abundance of all organisms of similar nature in a certain community. In an aspect, relative abundance refers to the abundance of cells of a particular strain of bacteria relative to the abundance of all bacteria of the same species in a certain community. In an aspect, relative abundance refers to the abundance of cells of a particular strain of bacteria relative to the abundance of all bacterial cells in a community. In an aspect, relative abundance is measured by qPCR comparing PCR products generated with 16S primers targeting specific bacteria species (or strain) of interest against PCR products generated with universal primers targeting all 16S sequences. See e.g., Chu, N., et al., “Profiling living bacteria informs preparation of fecal microbiota transplantations.” PLoS One 12(1): 1-16 (2017). In another aspect, the relative abundance is measured based on the number of sequence reads detected via high-throughput sequencing. Unless specified otherwise, a bacterial relative abundance mentioned herein is measured via high-throughput sequencing. In a further aspect, propidium monoazide (PMA) is used to differentiate between viable and dead fecal microbes as shown in Chu et al., PLoS One 12(1): 1-16 (2017).

As used herein, the term “treating” refers to (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures.

As used herein, a “subject” refers to any animal subject including humans, laboratory animals (e.g., primates, rats, mice), livestock (e.g., cows, sheep, goats, pigs, turkeys, chickens), and household pets (e.g., dogs, cats, rodents, etc.). Preferred subjects are human subjects. The human subject may be a pediatric, adult or a geriatric subject. In some aspects, the terms “patient” and “subject” are used interchangeably.

In an aspect, “secondary bile acid deficiency”, “bile acid metabolism deficiency”, or “deficiency in bile acid metabolism” refers to a person unable to transform primary bile acids to secondary bile acids at a level equal to a healthy human subject. In another aspect, a subject in need of supplementation with bile acid transforming bacteria refers to a subject with a bile acid metabolism deficiency or a secondary bile acid deficiency.

In an aspect, a “bacterial clade” or “clade” refers to a group of bacteria or taxa that locate proximately on a taxonomic or phylogenetic tree and share one or more bile acid metabolism consensus sequences. In an aspect, a bacterial clade comprises bacteria that share a taxonomic relatedness based on a 16S comparison. In another aspect, a bacterial clade comprises a single family of bacteria. In yet another aspect, a bacterial clade comprises a single genus of bacteria. In another aspect, a bacterial clade is comprised of bacteria comprising one or more common bile acid metabolism consensus sequences and one or more bacteria species (or strains). In an aspect, a bacterial clade refers to all nodes, extinct and living, in a branch of a rooted phylogenetic tree. In an aspect, a bacterial clade is delimited by a shared common ancestor (i.e., an internal node of interest) and all its descendants in a phylogenetic tree, making it monophyletic. Terminal nodes in a clade represent current living descendants of their shared common ancestor, and internal nodes represent intermediates between the shared common ancestor and extant organisms.

As used herein, a “strain” refers to a variant of a species differing in some genetic, epigenetic and/or phenotypic aspect to an extent that is insufficient to result in a new species. As used herein, whenever a composition comprises a bacterial species, in certain aspects such species may be present in the composition in multiple strains or variants. Also as used herein, a composition can comprise strains of different bacterial species.

As used herein, a “stool” refers to solid matter or part thereof that is released by a mammal (e.g., human), typically due to a bowel movement.

As used herein, a “microbiota” and “flora” refer to a consortium of microbes that live in or on a subject's body, both sustainably and transiently, including eukaryotes, archaea, bacteria, and viruses (including bacterial viruses (i.e., phage)). A “fecal microbiota” or “fecal microbiota preparation” refers to a community of microbes present in or prepared from a subject's feces. Herein a “consortium of fecal bacteria” refers to a composition of multiple species (or strains) of bacteria that have been extracted or harvested together from a stool of a donor. Following extraction or harvesting from a stool, the multiple species or strains of bacteria in a consortium of fecal bacteria are not isolated or purified away from each other (although they may have been co-cultured) and hence each species or strains in a consortium of fecal bacteria traces a temporally continuous co-habitation or co-environment with every other species or strain in the consortium, from their presence together in a stool to their co-existence in a formulated pharmaceutical composition described herein. In one aspect, a fecal microbiota transplant (FMT) therapy includes a consortium of fecal bacteria.

In an aspect, a consortium of fecal bacteria comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least thirty, at least forty, at least fifty, at least sixty, at least seventy, at least eighty, at least ninety, at least one hundred, at least one hundred and fifty, at least two hundred, at least two hundred and fifty, at least three hundred, at least three hundred fifty, at least four hundred, at least four hundred and fifty, at least five hundred, at least six hundred, at least seven hundred, at least eight hundred, at least nine hundred, at least one thousand, or more than one thousand species or strains of fecal bacteria.

In an aspect, a consortium of fecal bacteria comprises at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least thirty, at least forty, at least fifty, at least sixty, at least seventy, at least eighty, at least ninety, at least one hundred, at least one hundred and fifty, at least two hundred, at least two hundred and fifty, at least three hundred, at least three hundred fifty, at least four hundred, at least four hundred and fifty, at least five hundred, at least six hundred, at least seven hundred, at least eight hundred, at least nine hundred, at least one thousand, or more than one thousand strains of a species of fecal bacteria. In another aspect, a consortium of fecal bacteria comprises a single strain of a species and excludes one or more strains of the same species. In an aspect, a consortium of fecal bacteria comprises strains of bacteria from different species. In an aspect, a consortium of fecal bacteria comprises strains of bacteria from different genera. In an aspect, a consortium of fecal bacteria comprises strains of bacteria from different families. In an aspect, a consortium of fecal bacteria comprises strains of bacteria from different orders. In an aspect, a consortium of fecal bacteria comprises strains of bacteria from different classes. In an aspect, a consortium of fecal bacteria comprises strains of bacteria from different phyla. In an aspect, a consortium of fecal bacteria comprises the complement of bacteria in a fecal microbiota. In an aspect, a consortium of fecal bacteria is in the form of a substantially complete fecal microbiota. Herein the term “substantially complete fecal microbiota” refers to a preparation that comprises viable bacterial cells from all or substantially all of the bacterial taxa represented among the viable bacterial cells in the stool from which the fecal microbiota was extracted. In an aspect, the relative abundance of viable bacterial cells from at least two of the taxa in the substantially complete fecal microbiota is proportional to the relative abundance of the viable cells from those taxa in the stool from which the fecal microbiota was extracted.

Typically, a consortium of fecal bacteria described herein is extracted from a stool or stools of a single human donor. In an aspect, a consortium of fecal bacteria from the stool of one donor is combined or blended with a consortium of fecal bacteria from the stool of a different donor to produce a composition comprising consortia of fecal bacteria.

In an aspect, a consortium of fecal bacteria comprises bacteria that have not been subject to culturing and as such comprise uncultured fecal bacteria. Herein “uncultured fecal bacteria” or a “preparation of uncultured fecal bacteria” refers to a mixture of bacteria extracted together from a stool, wherein the bacteria in the mixture have not been subjected to culturing or fermentation in artificial growth media. Herein such a preparation of uncultured fecal bacteria can also be referred to as a collection of uncultured fecal bacteria or a population or community of uncultured fecal bacteria.

In another aspect, a consortium of fecal bacteria comprises bacteria (e.g. bacterial strains) that have been subjected to culturing or fermentation. For example, a fecal microbiota can be mixed with a culturing or fermentation medium that facilitates the replication of bacterial cells of different species and optionally the maintenance over time during replication of the relative abundance of particular species relative to the relative abundance of such species in the fecal microbiota prior to culturing or fermentation.

In some aspects, a consortium of fecal bacteria from a fecal microbiota comprises non-selected fecal bacteria. Herein “non-selected fecal bacteria” refers to a collection of viable fecal bacteria (e.g., bacterial strains present in a fecal microbiota) extracted from one or more stools without subjecting the extracted bacteria to environmental conditions that intentionally select for a particular type, state or taxonomic category of bacteria (e.g., by deliberate removal of certain species of bacteria, treatment of the bacteria with an agent such as ethanol or chloroform, or culturing). Such non-selected fecal bacteria can comprise bacteria in proportional content to corresponding bacteria in a fecal or intestinal microbiota of a normal healthy human. Steps taken to non-selectively extract fecal bacteria from a stool or portion thereof can include, for example, homogenization and filtering of the stool or portion thereof to separate the fecal bacteria from non-cellular stool material such as fiber and rough particulate matter, as well as, for example, eukaryotic host cells and viruses. Herein typically a non-selected fecal bacterial preparation can be prepared in either aerobic or anaerobic conditions, or a combination thereof. In certain aspects, a preparation of non-selected fecal bacteria comprises all or substantially all of the bacteria of a fecal microbiota of a stool or portion thereof. In certain aspects, a preparation of non-selected fecal bacteria comprises all or substantially all of the species of a fecal microbiota of a stool or portion thereof. In certain aspects, a preparation of non-selected fecal bacteria comprises all or substantially all of the bacterial species or strains of a fecal microbiota of a stool or portion thereof. In certain aspects, a preparation of non-selected fecal bacteria comprises all or substantially all of the bacterial genera of a fecal microbiota of a stool or a portion thereof. In certain aspects, a preparation of non-selected fecal bacteria comprises all or substantially all of the bacterial phyla of a fecal microbiota of a stool or portion thereof. Therefore, such non-selective fecal microbiota can substantially resemble microbial constituents and the bacterial population structure found in such fecal sample. In an aspect, a consortium of fecal bacteria comprises at least 2, 5, 10, 20, 30, 40, 50, 100, 200, 300, 400, 500, or 600 bacterial species or strains. In another aspect, a consortium of fecal bacteria comprises between 2 and 5, 5 and 10, 10 and 20, 20 and 30, 30 and 40, 40 and 50, 50 and 60, 60 and 100, 100 and 200, 200 and 300, 300 and 400, 400 and 500, or 500 and 600 bacterial species or strains.

In an aspect, a consortium of fecal bacteria (e.g., a preparation of uncultured fecal bacteria and/or a preparation of non-selected fecal bacteria) does not comprise an antibiotic resistant population of bacteria.

In another aspect, the preparation of a composition comprising a consortium of fecal bacteria can involve steps that select for a particular, type, state, or taxonomic category of bacteria (e.g., by deliberate removal of certain strain or species of bacteria, treatment of the population with a selective agent such as ethanol or chloroform, culturing the bacteria in selective media and/or screening of the bacteria for the ability to produce a metabolite at or above a threshold level).

Herein a consortium of fecal bacteria is distinguished from an isolated or purified strain or species of bacteria. As used herein, “bacterial isolate” refers to a collection of isolated, substantially genetically identical bacterial cells generated by proliferation via binary fission from a single predecessor bacterial cell (e.g., by culturing the bacteria). Typically, a bacterial isolate is originally isolated as a single cell or genetically pure group of cells, for example, as a single colony on solid culture media or via serial dilutions in liquid culture, and thereafter archived (e.g. as a frozen stock) to provide a consistent and stable source for the isolate. Once isolated, in some aspects, a bacterial isolate can be grown as a pure culture of cells; in other aspects, multiple bacterial isolates can be grown simultaneously in the same vessel as a mixed culture. The term “substantially genetically identical” refers to the very high (e.g. >99.9%) genetic identity shared by different cells in uncontaminated pure compositions of bacterial isolates, owing to their proliferation from a common predecessor, but accounts for minor genetic dissimilarity between cells due to accumulations of relatively rare mutations. Generally, a bacterial isolate is synonymous with a pure culture of bacterial cells. Typically, herein a bacterial isolate consists of non-pathogenic bacteria. In an aspect, a bacterial isolate can be a probiotic, or an ingredient in a probiotic.

As used herein, the term “culture” or “culturing” refers to the growth of cells in an artificial medium that is designed to facilitate the expansion, reproduction or propagation of microbial cells. Without limitation, culturing can occur in a liquid, semi-solid, or solid growth medium, such as broth or agar. Growth medium typically contains appropriate nutrients for the growth of the microbial cells.

As used herein, the term “bacterial cocktail”, sometimes called a “bacterial consortium” or “synthetic bacterial mixture”, refers to an engineered mixture of bacteria comprising a defined consortium of multiple bacterial isolates. The term “defined consortium of multiple bacterial isolates” means that the bacterial cocktail contains two or more bacterial isolates, and that the identity of each bacterial isolate in the cocktail is known, and thus the cocktail can be consistently produced (e.g. by combining isolated bacterial strain or species) to have a stable composition and properties across separate batches. Herein “identity” of a bacterial isolate can refer to any characteristic of the isolate that uniquely identifies the isolate as different from one or more other bacterial isolates or bacterial strain or species. Examples of identifying characteristics of a bacterial isolate include nucleotide sequences such as a 16S rRNA sequence, the sequence of one or more coding or non-coding regions of a nucleic acid, and entire genome sequences, level of gene expression, physiological or metabolic traits, or anatomical traits such as staining pattern or cell wall characteristics.

As used herein, “bacterial mixture” refers to an engineered composition comprising viable bacterial cells. In some aspects, a bacterial mixture comprises one or more non-pathogenic bacterial isolates. In some aspects, a bacterial mixture comprises a consortium of fecal bacteria. In some aspects, a bacterial mixture comprises both of one or more non-pathogenic bacterial isolates and a consortium of fecal bacteria. In another aspect, a bacterial mixture comprises a consortium of fecal bacteria from one or more donors.

As used herein, “therapeutically effective amount,” “effective amount” or “pharmaceutically active dose” refers to an amount of a composition which is effective in treating the named disease, disorder, condition, or symptom.

As used herein, “isolated” or “purified” refers to a bacterium or other entity or substance that has been (1) separated from at least some of the components with which it was associated when initially produced (whether it was initially produced in nature or in an experimental setting), and/or (2) produced, prepared, purified, and/or manufactured by the hand of man. Isolated or purified bacteria can be separated from at least about 10%, about 20%, about 30%, about 40%, about 50%, about 60%, about 70%, about 80%, about 90%, or more of the other components with which they were initially associated.

As used herein, the terms “non-pathogenic” in reference to a bacterium or any other organism or entity includes any such organism or entity that is not capable of causing or affecting a disease, disorder or condition of a host organism containing the organism or entity.

As used herein, “spore” or a population of “spores” includes bacteria (or other single-celled organisms) that are generally viable, more resistant to environmental influences such as heat and bactericidal agents than vegetative forms of the same bacteria, and typically capable of germination and out-growth. “Spore-formers” or bacteria “capable of forming spores” are those bacteria containing the genes and other necessary abilities to produce spores under suitable environmental conditions.

As used herein, “colony forming units” (CFUs) refers to an estimate of the number of viable microorganism cells in a given sample. The number of CFUs can be assessed by counting the number of colonies on an agar plate as in standard methods for determining the number of viable bacterial cells in a sample.

As used herein, “viable” means possessing the ability to multiply. The viability of bacterial populations can be monitored as a function of the membrane integrity of the cell. Cells with a compromised membrane are considered to be dead or dying, whereas cells with an intact membrane are considered live. For example, SYTO 9 and propidium iodide are used to stain and differentiate live and dead bacteria. See Stocks, Cytometry A. 2004 October; 61(2):189-95. Cell viability can also be evaluated via molecular viability analyses, e.g., a PCR-based approach, which can differentiate nucleic acids associated with viable cells from those associated with inactivated cells. See Cangelosi and Mescheke, Appl Environ Microbiol. 2014 October; 80(19): 5884-5891.

As used herein, “Shannon Diversity Index” refers to a diversity index that accounts for abundance and evenness of species present in a given community using the formula H=−Σᵢ₌₁^{R}pᵢ ln pᵢ, where H is Shannon Diversity Index, R is the total number of species in the community, and pᵢ is the proportion of R made up of the ith species. Higher values indicate diverse and equally distributed communities, and a value of 0 indicates only one species is present in a given community. For further reference, see Shannon and Weaver, (1949) The mathematical theory of communication. The University of Illinois Press, Urbana. 117 pp.

As used herein, “antibiotic” refers to a substance that is used to treat and/or prevent bacterial infection by killing bacteria, inhibiting the growth of bacteria, or reducing the viability of bacteria.

Disclosed herein is a pharmaceutical composition that comprises a bacterial mixture comprising a bacterial isolate selected for inclusion in the pharmaceutical composition on the basis of a bile acid transforming ability. In an aspect, the bacterial isolate can be selected based on an ability to transform a primary bile acid to a secondary bile acid. In another aspect, the bacterial isolate can be selected based on an ability to transform a secondary bile acid to a different secondary bile acid. Throughout the disclosure bile acid transforming bacteria and bile acid metabolizing bacteria is used interchangeably.

Secondary bile acids are produced from primary bile acids in the guts of humans and other mammals solely due to the activity of microbes. In an aspect, “primary bile acid transforming bacteria” is in reference to bacteria, a consortium of fecal bacteria, bacterial taxa, bacterial clades or bacterial isolates or strains refers to one or more bacteria (e.g., in the form of one or more bacterial isolates or strains) capable of transforming a primary bile acid to a secondary bile acid. Without wishing to be bound by theory, primary bile acids are synthesized in the liver from cholesterol in a process that constitutes the major pathway for cholesterol degradation. After synthesis, bile acid is stored in the gallbladder and later secreted into the duodenum. Bile is important for emulsification of dietary lipids, digestion and absorption of fatty acids, cholesterol, fat-soluble vitamins, and other hydrophobic diet components. A majority of secreted bile acids are reabsorbed in the small intestine. Bile acids that are not absorbed in the small intestine migrate to the large intestine where they are normally metabolized by the microbiota, before being reabsorbed from the colon. Fiamoncini, J., The Cross Talk Between Bile Acids and intestinal Microbiota, Microbiome and Metabolome in Diagnosis, Therapy, and other Strategic Applications. (2019). Deoxycholic acid (DCA), lithocholic acid (LCA), and ursodeoxycholic acid (UDCA) are the most common secondary bile acids formed through bacterial metabolism of primary bile acid in the human intestines. For example, gut bacteria can perform C24 amide hydrolysis and 7α-dehydroxylation on primary bile acids, transforming the glycine and taurine conjugates of CA and CDCA into their corresponding secondary bile acids deoxycholic acid (DCA) and lithocholic acid (LCA), respectively. Although DCA and LCA are typically the most abundant secondary bile acids, ˜50 different secondary bile acids exist in human stool. Herein a secondary bile acid “corresponds to” a primary bile acid if the secondary bile acid can be produced from the primary bile acid via the metabolism of intestinal primary bile acid transforming bacteria. For example, the secondary bile acid DCA corresponds to the primary bile acid CA, and secondary bile acid LCA corresponds to the primary bile acid CDCA. Likewise, a first secondary bile acid “corresponds to” a second secondary bile acid if the second secondary bile acid can be produced from the first secondary bile acid via the metabolism of intestinal secondary bile acid converting bacteria. For example, the secondary bile acids IDCA, GDCA and TDCA correspond to the secondary bile acid DCA, and the secondary bile acids GLCA, TLCA and UDCA correspond to the secondary bile acid LCA.

In an aspect, the compositions disclosed herein provide for bile acid transformations in a subject administered the compositions as provided in Table 1.

TABLE 1

| | Secondary | |
|---|---|---|
| Primary Bile Acid | Bile Acid 1 | Secondary Bile Acid 2 |
| Cholic acid (CA) | Deoxycholic | Isodeoxycholic acid (IDCA) |
| | acid (DCA) | Glycodeoxycholic acid (GDCA) |
| | | Taurodeoxycholic acid (TDCA) |
| Chenodeoxycholic | Lithocholic | Glycolithocholic acid (GLCA) |
| acid (CDCA) | acid (LCA) | Taurolithocholic acid (TLCA) |
| | | Ursodeoxycholic acid (UDCA) |

In an aspect, primary bile acid transforming bacteria administered to a subject in a composition described herein convert the primary bile acid cholic acid to the secondary bile acid deoxycholic acid (DCA) and/or the primary bile acid chenodeoxycholic acid to the secondary bile acid lithocholic acid (LCA). In an aspect, secondary bile acid converting bacteria administered to a subject in a composition described herein convert the secondary bile acid DCA to one or more of the secondary bile acids isodeoxycholic acid (IDCA), glycodeoxycholic acid (GDCA) and/or taurodeoxycholic acid (TDCA). In an aspect, secondary bile acid converting bacteria administered to a subject in a composition described herein convert the secondary bile acid LCA to one or more of the secondary bile acids glycolithocholic acid (GLCA), taurolithocholic acid (TLCA), and/or usrodeoxycholic acid (UDCA). In certain aspects, the secondary bile acid converting bacteria can further convert UDCA to isoursodeoxycholic acid and/or glycoursodeoxycholic acid (GUDCA) and/or tauroursodeoxycholic acid (TUDCA).

In certain other aspects, primary acid transforming bacteria can transform the primary bile acid cholic acid to one or more of the secondary bile acids 12-oxoLCA, 3-oxoCA or 7-oxoDCA, and/or transform the primary bile acid CDCA to one or more of the secondary bile acids 3-oxoLCA, 3-oxoCDCA or 7-oxoLCA. In another aspect, secondary bile acid converting bacteria can convert the secondary bile acid LCA to 3-oxoLCA. In another aspect, secondary bile acid converting bacteria can convert the secondary bile acid 3-oxoLCA to one or more of the secondary bile acids isoLCA, isoalloLCA and/or 3-oxoalloLCA. In still another aspect, secondary bile acid bacteria can convert 3-oxoalloLCA to isoalloLCA or DCA to isoDCA.

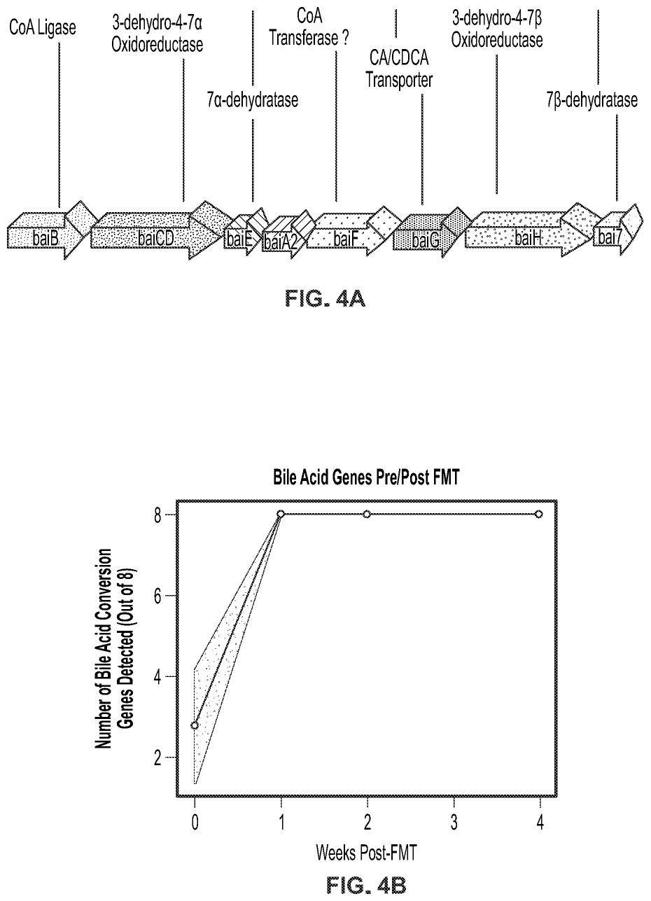

In an aspect, the transformation of a primary bile acid to a secondary bile acid by primary bile acid transforming bacteria is mediated by one or more genes of the bile acid inducing (bai) gene cluster or operon (see FIG. 4A). In an aspect, this cluster of genes encodes seven enzymes and one transporter that carry out bile acid metabolism in gut bacteria capable of transforming a primary bile acid to a secondary bile acid. In an aspect, “bai gene” or “bai genes” refers to one or more of the following bile acid transforming pathway genes: baiB encoding a CoA ligase; baiCD encoding a 3-dehydro-4-7a oxidoreductase; baiE encoding a 7α-dehydratase; baiA1/A2 encoding a 3α-Hydroxysteroid Dehydrogenase; baiF encoding a coenzyme A (CoA) hydrolase; baiG encoding a bile acid transporter; baiH encoding a NADH:flavin oxidoreductase; and baiI encoding a ketosteroid isomerase. In an aspect, the bai genes are present in bacterial DNA in a cluster or operon (e.g., bile acid inducible operon); herein “bai operon” refers to a collection of genes capable of mediating the transformation of a primary to a secondary bile acid when activated and/or transcribed.

In an aspect, “secondary bile acid converting bacteria” in reference to bacteria, a consortium of fecal bacteria, bacterial taxa, bacterial clades or bacterial isolates refers to one or more bacterial (e.g., in the form of one or more bacterial isolates or strain) that encode an enzyme required for the metabolism of one secondary bile acid to a different secondary bile acid. In an aspect, the bioconversion of one secondary bile acid to a different secondary bile acid is a cooperative process in which multiple bacterial strains or species possess different genetic and/or enzymatic components of an overall metabolic pathway that converts a secondary bile acid to a different secondary bile acid. In such a case, individual cells harboring the genetic or metabolic capability for carrying out a particular step in the secondary bile acid transforming pathway would typically secrete the product of their metabolic step into the surrounding microbiota matrix, where such intermediate would be taken up by a different cell capable of performing the subsequent step(s) in the metabolic pathway.

The term “bile acid transforming bacteria” as used herein refers to both primary bile acid transforming bacteria and secondary bile acid converting bacteria. Without wishing to be bound by theory, bacterially produced bile acids can influence host physiology and health in a variety of ways. For example, in certain aspects secondary bile acids can be ligands for the farnesoid X receptor (FXR), the liver X receptor (LXR), the G-protein coupled receptor TGR5, and the vitamin D receptor, and thereby initiate a variety of signaling pathways relevant to hepatic and metabolic disorders. DCA production has further been shown to limit the outgrowth of the enteric pathogen Clostridium difficile, and UDCA protects against the development of chemically induced colon cancer in animal studies and can be used to treat gallstones.

The absence or low relative abundance of certain bile acid transforming bacteria in the intestinal microbiota of a subject relative to a healthy subject can result in a reduced abundance or rate of production of secondary bile acids, which can cause or exacerbate an intestinal dysbiosis in the subject. In an aspect, a subject in need of treatment with a pharmaceutical composition described herein may have a gut dysbiosis, and/or a disease, disorder or condition related to or arising from a gut dysbiosis, that is associated with a reduced concentration, amount or rate of production of one or more secondary bile acids in the intestine of the subject.

In an aspect, a subject in need of treatment with a composition described herein has a deficient intestinal concentration or rate of production of one or more secondary bile acids as a result of a reduced relative abundance of one or more bile acid transforming strains or species in the intestine of the subject relative to a healthy individual (e.g., one or more strains or species of primary bile acid transforming bacteria or secondary bile acid converting bacteria). For example, primary bile acid transforming bacteria may be deficient (e.g., reduced in relative abundance relative to an individual that does not have a gut dysbiosis) or absent in the intestine of a subject in need of treatment with a composition described herein. In another example, secondary bile acid converting bacteria required to catalyze a particular step in the overall metabolic pathway that converts one secondary bile acid to a different secondary bile acid may be deficient or absent in the intestine of a subject in need of treatment with a composition described herein. As a result of such deficiency in or absence of certain bile acid transforming bacteria (e.g., strain) in the intestine of a subject, such subject may exhibit a block or gap in bile acid metabolism that prevents the subject from producing one or more secondary bile acids beneficial to the health of the subject. The compositions described herein are designed to introduce into the gut of a patient the necessary metabolic potential to rescue the block or gap in bile acid metabolism, in the form of viable bile acid transforming bacteria.

In an aspect, the absence of a bile acid transforming bacterial clade in a microbiome of a subject can be considered a gene deletion that may be restored or corrected via gene therapy. In an aspect, gene therapy comprises cell therapy. In an aspect, gene therapy comprises the administration of a composition as described herein. In an aspect, gene therapy comprises the administration of a composition comprising bile acid transforming bacteria. In an aspect, gene therapy comprises the administration of a microbial therapeutic as described herein. In an aspect, gene therapy comprises the administration of a microbial therapeutic comprising bile acid transforming bacteria.

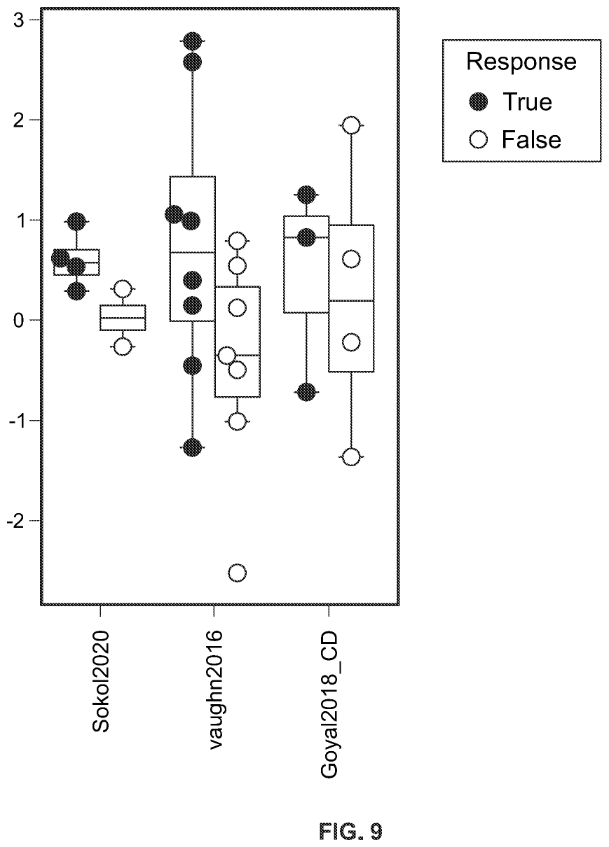

In an aspect, disclosed herein is a method comprising determining that a fecal microbiota of a subject comprises a number of clades of bacteria comprising a bai gene operon that is less than a threshold value, and administering to the subject a bacterial mixture comprising bacteria harboring the bai gene operon. In certain aspects, the subject cacks bacteria containing the bai gene operon. In certain aspects, such a “bai gene deletion” explains or is the cause of an intestinal-related pathology in the subject, for example a pathology that exhibits intestinal symptoms (e.g., inflammatory bowel disease), and administration of the bacteria comprising the bai gene operon is capable of treating the pathology. Therefore, the compositions and methods disclosed herein can be used as a precision medicine strategy that treats a patient deficient for a gene or gene module via microbial gene therapy to deliver the missing gene or gene module to the patient inside of viable bacteria. In an aspect, the viable bacteria administered to the subject have not been genetically engineered. In an aspect, the viable bacteria are capable of engrafting in the intestine of the subject, where they actively transform bile acids to produce secondary bile acids of use to the subject in alleviating the symptoms of the pathology caused by the “deletion” of the gene or gene module. Results demonstrating the success of this gene therapy approach are in FIG. 9, which shows that Crohn's disease patients who respond to treatment with an uncultured consortium of fecal bacteria (FMT) consistently show an increase in the abundance of the bai gene operon post-treatment compared to non-responders.

In an aspect, disclosed herein is a method comprising determining that a fecal microbiota of a subject comprises a number of clades of bacteria comprising isoalloLCA producing genes that is less than a threshold value, and administering to the subject a bacterial mixture comprising bacteria harboring the isoalloLCA producing genes. In an aspect, the isoalloLCA producing genes comprise one or more genes selected from the group consisting of a 5β-reductase, 5α-reductase, and 3β-hydroxy-delta5-C27-steroid dehydrogenase (3β-HSDH). In an aspect, bacteria harboring the isoalloLCA producing genes comprises bacteria from one or more genus selected from the group consisting of Bacteroides, Parabacteroides, Butyricimonas, Odoribacter, Porphyromonas, Alistipes, and a combination thereof. In an aspect, bacteria harboring the isoalloLCA producing genes comprises bacteria from one or more species selected from the group consisting of Bacteroides uniformis, Bacteroides finegoldii, Bacteroides fragilis, Bacteroides dorei, Bacteroides vulgatus, Bacteroides caccae, Bacteroides ovatus, Bacteroides eggerthii, Bacteroides thetaiotaomicron, Parabacteroides goldsteinii, Parabacteroides distasonis, Parabacteroides merdae, Butvricimonas synergistica, Odoribacter laneus, Porphyromonas somerae, Alistipes indistinctus, Alistipes onderdonkii, Alistipes finegoldii, and a combination thereof.

A deficiency in the amount or rate of production of one or more secondary bile acids produced in the intestine of a subject due to the deficiency or absence of certain bile acid transforming bacteria (e.g., primary bile acid transforming species or strain, or secondary bile acid converting species or strain) is to be distinguished from a reduced abundance of secondary bile acids arising directly from non-microbiota host factors. Examples of such non-microbiota factors include host-provided or host-encoded cellular components that impact the absorption, activity, rate of metabolism and/or downstream conversion of secondary bile acids produced by the microbiota. Disorders or conditions arising from variability in such host-provided mechanisms can thus be distinguished from intestinal dysbiosis and related disorders arising directly from a deficiency in the composition of the host microbiota, i.e., a deficiency in second bile acid production due to reduced relative abundance or activity of bile acid transforming bacteria. It is thus desirable to distinguish in practice between a situation where a patient is susceptible to or suffers from a condition or disorder due to a deficiency in bile acid transforming bacteria, and a patient whose disorder arises from a defect or deficiency in non-microbiota host-provided factors. The application of the methods disclosed herein provide for a means to identify patients or a patient population having deficiencies in the relative abundance or activity of intestinal bile acid transforming bacteria, thereby identifying such patient or patient population as a candidate likely to benefit from treatment with a composition comprising one or more bile acid transforming species or strain, for example administered as a bacterial mixture comprising a bacterial isolate capable of rescuing the microbiota-based bile acid transforming deficiency (e.g., due to the presence in such bacterial isolate of bai genes).

Accordingly, disclosed herein is a method of selecting a patient or patient population who may benefit from the introduction of bile acid transforming bacteria into the intestine, the method comprising identifying a deficiency in bile acid metabolism in the patient or patient population, and selecting the patient or patient population for treatment. As described further below, non-limiting means to identify a deficiency in bile acid metabolism that can be used as a basis to select subjects or populations of subjects as candidates to receive a treatment described herein include the absence or low relative abundance of bacterial clades comprising a bile acid metabolism consensus sequence; the production of a secondary bile acid (from a corresponding primary bile acid or a corresponding secondary bile acid) in a functional assay at a level or rate that is lower than a threshold level or rate (e.g., determined by measuring a ratio of the secondary to primary bile acids before and after the assay); the determination that a secondary bile acid is present in stool at a level lower than a threshold level, a primary bile acid is present in stool at a level higher than a threshold level, or a ratio of a secondary bile acid to a primary bile acid in stool is at a level lower than a threshold level; or a combination thereof.

In an aspect, a method of selecting a subject who may benefit from a treatment disclosed herein comprises screening a consortium of fecal bacteria (e.g., in the form of a fecal microbiota) for a deficiency in bile acid metabolism, and selecting the individual as the subject on the basis of determining that the consortium of fecal bacteria is deficient in bile acid metabolism. Herein “screening” a consortium of fecal bacteria for a deficiency in bile acid metabolism refers to the application or implementation of a test, assay or method (e.g., a computational method that analyzes metagenomics sequencing data) capable of quantifying or qualitatively describing a capacity of the consortium to transform a primary bile acid to a secondary bile acid or convert a secondary bile acid to a different secondary bile acid.

An example of a method that can identify or select a subject or population of subjects who may benefit from the treatments described herein comprises analyzing metagenomics data from a consortium of fecal bacteria of an individual for one or more bile acid metabolism consensus sequences (e.g., genes) known to mediate or be associated with one or more steps in a metabolic pathway that transforms a primary to a secondary bile acid or converts a first secondary bile acid to a second secondary bile acid. Herein the term “bile acid metabolism consensus sequence” refers to any nucleotide sequence (e.g., DNA sequence) that predicts and/or correlates with an ability of a bacterial strain, bacteria cell of a species, or clade comprising the consensus sequence to catalyze a step in a metabolic pathway that produces a secondary bile acid (i.e., from a primary bile acid or a secondary bile acid). In an aspect, such a bile acid metabolism consensus sequence is a DNA sequence comprising one or more genes or a regulatory region for one or more genes known to encode a product or products that act in a metabolic pathway functioning to transform a primary bile acid to a secondary bile acid. In an aspect, the bile acid metabolism consensus sequence is adjacent to a DNA sequence encoding a gene product that acts in a bile acid metabolic pathway. Herein “adjacent” refers to less than 100 bp, less than 200 bp, less than 300 bp, less than 400 bp, less than 500 bp, less than 1 kbp, less than 2 kbp, less than 3 kbp, less than 4 kbp, less than 5 kbp, less than 10 kbp, less than 15 kbp, less than 20 kbp, less than 30 kbp, less than 40 kbp, less than 50 kbp, or less than 100 kbp from the start of a gene encoding a product acting in a bile acid metabolic pathway. In an aspect, the consensus sequence comprises one or more bai genes or a regulatory region for one or more bai genes. In an aspect, the consensus sequence comprises at least one, at least two, at least three, at least four, at least five, at least six, at least seven, or at least eight bai genes. In an aspect, the consensus sequence comprises the bai operon. In an aspect, a bile acid metabolism consensus sequence does not encode a gene but associates with or is linked to or indicative of the presence of a gene or genes that produce one or more gene products that catalyze a step in a metabolic pathway that converts a primary bile acid to a secondary bile acid or a first secondary bile acid to a second secondary bile acid. In an aspect, a consensus sequence described herein includes all or a portion of a gene (e.g., 5β-reductase, 5α-reductase, 3β-HSDH, or a bai gene)

In another aspect, a bile acid metabolism consensus sequence refers to any amino acid sequence that predicts the ability of a bacterial strain, bacteria cell of a species or clade comprising the consensus sequence to catalyze a step in a metabolic pathway that produces a secondary bile acid (i.e., from a primary bile acid or a secondary bile acid). In another aspect, a bile acid metabolism consensus sequence refers to any amino acid sequence that correlates with an ability of a bacterial species, bacterial strain, or clade comprising the consensus sequence to catalyze a step in a metabolic pathway that produces a secondary bile acid (i.e., from a primary bile acid or a secondary bile acid). In another aspect, a bile acid metabolism consensus sequence comprises an amino acid sequence produced by one or more genes known to encode a product that acts in a metabolic pathway functioning to convert a first secondary bile acid to a second secondary bile acid. In an aspect, a bile acid consensus sequence comprises at least 7 amino acids, at least 20 amino acids, at least 40 amino acids, or at least 60 amino acids.

In an aspect, a bile acid metabolism consensus sequence comprises at least 20 bp, at least 30 bp, at least 40 bp, at least 50 bp, at least 100 bp, at least 200 bp, at least 300 bp, at least 400 bp, at least 500 bp, at least 1 kbp, at least 2 kbp, at least 3 kbp, at least 4 kbp, at least 5 kbp, at least 10 kbp, at least 20 kbp, at least 30 kbp, at least 40 kbp or at least 50 kbp. In an aspect, a bile acid metabolism consensus sequence comprises or consists of a sequence that is targeted by a primer in a PCR reaction, such that the addition of two primers produce a product in the PCR reaction.

In another aspect, a bile acid metabolism consensus sequence comprises a DNA sequence indicative of or comprising or adjacent to one or more genes or a regulatory region for one or more genes known to encode a product that acts in a metabolic pathway functioning to convert a first secondary bile acid to a second secondary bile acid. In an aspect, the bile acid metabolism consensus sequence is indicative of or comprises or is adjacent to a gene encoding 3α-hydroxysteroid dehydrogenase, which can convert the secondary bile acid LCA into the secondary bile acid 3-oxoLCA. In another aspect, the bile acid metabolism consensus sequence is indicative of or comprises or is adjacent to a gene encoding 3β-hydroxysteroid dehydrogenase, which can convert the secondary bile acid 3-oxoLCA into the secondary bile acid isoLCA. In still another aspect, the bile acid metabolism consensus sequence is indicative of or comprises or is adjacent to one or more genes selected from the group consisting of a 5β-reductase, 5α-reductase, and 3β-HSDH, which together can convert the secondary bile acid 3-oxoLCA to the secondary bile acid isoalloLCA. In an aspect, the bile acid metabolism consensus sequence comprises a cluster of genes comprising the 5β-reductase, 5α-reductase, and 3β-hydroxysteroid dehydrogenase, or portions thereof. In an aspect, the bile acid metabolism consensus sequence comprises a 5β-reductase gene or a portion thereof. In an aspect, the bile acid metabolism consensus sequence comprises a 5α-reductase gene or a portion thereof. In an aspect, the bile acid metabolism consensus sequence comprises a 3β-hydroxysteroid dehydrogenase gene or a portion thereof. In an aspect, a consensus sequence described herein includes all or a portion of a gene (e.g., 5β-reductase, 5α-reductase, 3β-HSDH, or a bai gene)

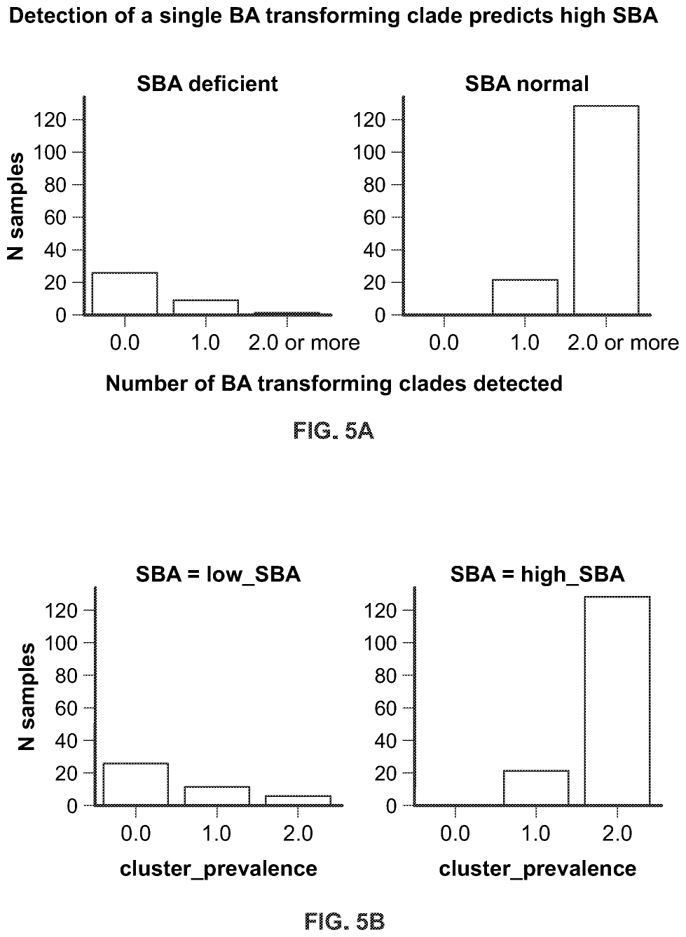

Disclosed herein is a method comprising determining that a consortium of fecal bacteria of an individual either lacks a particular bile acid metabolism consensus sequence or has a number of clades of bacteria comprising a bile acid metabolism sequence less than a threshold number of clades, and selecting such individual for a treatment described herein or as a candidate to benefit from a treatment described herein. Typically, the absence or deficient number of bacterial clades comprising a particular bile acid metabolism consensus sequence in the intestine of an individual correlates with or predicts a deficiency in the level of a secondary bile acid or the proportion or ratio of secondary bile acid to its corresponding primary or secondary bile acid. For example, individuals having 0 clades of bacteria comprising a bai gene operon show a ratio of DCA:CA of less than 1 (FIG. 5A) and a LCA:CDCA ratio of less than 1 (FIG. 5B), whereas a single bacterial clade comprising the operon in the intestine of a subject can be sufficient to normalize the DCA:CA ratio (FIG. 5A) and LCA:CDCA ratio (FIG. 5B) above 1, and two or more bacterial clades comprising a bai gene operon highly correlates with the correction of the deficiency in DCA:CA (FIG. 5A) and LCA:CDCA (FIG. 5B). In aspect, an absence or deficiency in the number of bacterial clades comprising bai genes or a bai gene operon correlates with a ratio of secondary bile acid to corresponding primary bile acid of less than 1 (e.g., DCA:CA<1 or LCA:CDCA <1.

It will be understood that different bile acid metabolism consensus sequences can mark or indicate different functions in the transformation from a primary to secondary bile acid or the conversion between two bile acids, and that an absence or reduction in number (below a threshold level) of different consensus sequences can lead to deficiencies in different secondary bile acids. For example, the absence or deficiency of intestinal bacteria comprising a consensus sequence that marks a functional bai operon encoding proteins (transporter and enzymes) necessary to convert CA to DCA is indicative of a microbiome-based error in metabolism manifested in deficient (or no) production of DCA in the gut, and a corresponding reduction in availability of secondary bile acids for the host. Such deficiency can be rescued or cured by introducing into the microbiome one or more bacterial strains, species or isolates that comprise a functional copy of the bai gene or genes that are missing (or an entire bai operon). Such introduction restores the gene products necessary to metabolize a bile acid substrate (in the case of DCA-producing genes, CA) thereby resolving the metabolic blockage and providing the host with the missing secondary bile acid (here DCA). In another aspect, a patient's microbiome may comprise sufficient primary bile acid transforming clades to convert primary bile acids, but a block in downstream secondary bile acid metabolism (e.g., indicated by an absence or reduced number of bacterial clades having a particular secondary bile acid metabolism consensus sequence) may result in an inability to convert a secondary bile acid to a different secondary bile acid. For example, the absence of a clade of bacteria comprising 3α-hydroxysteroid dehydrogenase can result in a deficiency in the secondary bile acid 3-oxoLCA and/or a decrease in the ratio of 3-oxoLCA:LCA. In another example, the absence of a clade of bacteria comprising 3β-hydroxysteroid dehydrogenase can result in a deficiency in the secondary bile acid isoLCA and/or a decrease in the ratio of isoLCA:3-oxoLCA. In still another example, the absence of a clade of bacteria comprising one or more of 5β-reductase, 5α-reductase and 3β-HSDH can result in a deficiency in the secondary bile acid isoalloLCA and/or a decrease in the ratio of isoalloLCA:3-oxoLCA.

Disclosed herein is a method of treating a variety of conditions, diseases and disorders related to a bile acid deficiency in a patient by complementing or rescuing a genetic defect in the microbiome of the patient that prevents the metabolism of a primary or secondary bile acid into a secondary bile acid product, thereby providing the host with the deficient secondary bile acid product. Such rescue of the bile acid deficient phenotype does not require genetic engineering. Instead, the methods herein advantageously provide for the absent or deficient metabolic activity by administering bacteria that naturally comprise a DNA sequence encoding a gene or gene products (or amino acid sequence produced by a gene) that are capable of performing the needed metabolic function. The administration of natural bacteria increases the probability that a particular administered species will successfully compete and/or engraft in the gut of the subject and thereby establish a threshold relative abundance necessary to take up the upstream bile acid substrate and provide the host with the missing or deficient product. Disclosed herein are compositions providing for bacteria that can restore a bile acid metabolism pathway at any step where function is missing. For example, a composition can provide for bacteria that provide transporter or enzyme activity to convert a first bile acid to a second bile acid, a first bile acid to a bile acid intermediate, a bile acid intermediate to a second bile acid, or a bile acid intermediate substrate to a bile acid intermediate product. In an aspect, a composition provides for bacteria comprising a gene encoding a transporter for importing a first bile acid or bile acid intermediate into the cell. In another aspect, a composition provides for bacteria comprising a gene encoding an exporter for exporting a second bile acid or a bile acid intermediate product out of the cell. In another aspect, a composition provides for bacteria comprising a gene encoding an enzyme that acts in a metabolic pathway that transforms a primary bile acid to a secondary bile acid, or a first secondary bile acid to a second secondary bile acid. In an aspect, a bacterial strain or species (e.g., administered in the context of a consortium of fecal bacteria) or a bacterial isolate administered to a patient comprises one or more genes encoding the following products to restore a deficiency in such product (e.g., in the event that a patient is determined to have a number of bacterial clades comprising a consensus sequence below a threshold value, where the consensus sequence is indicative of the presence of the gene or another gene acting in the same metabolic pathway): a CoA ligase (e.g., baiB gene function to restore a deficiency in primary bile acid transformation); 3-dehydro-4-7a oxidoreductase (e.g., baiCD gene function to restore a deficiency in primary bile acid transformation); 7α-dehydratase (e.g., baiE gene function to restore a deficiency in primary bile acid transformation); 3α-Hydroxysteroid Dehydrogenase (e.g., baiA1/A2 gene function to restore a deficiency in primary bile acid transformation); a coenzyme A (CoA) hydrolase (e.g., baiF gene function to restore a deficiency in primary bile acid transformation); a bile acid transporter (e.g., baiG gene function to restore a deficiency in primary bile acid transformation); NADH:flavin oxidoreductase (e.g., baiH gene function to restore a deficiency in primary bile acid transformation); and a ketosteroid isomerase 3α-hydroxysteroid dehydrogenase (e.g., baiI gene function to restore a deficiency in primary bile acid transformation), 3β-hydroxysteroid dehydrogenase to restore a deficiency in the metabolism of 3-oxoLCA to isoLCA; or one or more of 5β-reductase 5α-reductase or 3β-HSDH to restore a deficiency in the metabolism of 3-oxoLCA to isoalloLCA.

In an aspect, multiple bacterial strains or species (e.g., isolates) that provide for the same metabolic function are co-administered to a patient (e.g., in the same composition or separate compositions), thus conferring functional redundancy in the administered composition(s), in the event that one of the bacterial strains or species engrafts in the gut of the patient more efficiently or provides the missing or deficient metabolic function at a higher level in the context of the microbiome of a specific patient. In another aspect, multiple strains species of bacteria are administered to a patient to provide for different bile acid metabolic functions in the gut of the patient (e.g., one or more bacterial isolates comprising bai genes and/or one or more bacterial isolates comprising a gene encoding, e.g., 3β-hydroxysteroid dehydrogenase or one or more of 5β-reductase, 5α-reductase or 3β-HSDH). In an aspect, the bacterial strains, species or isolate includes bacteria from one or more genus of Clostridium, Bacillus, Bacteroides, Bifidobacterium, Catenibacterium, Collinsella, Eggerthella, Lachnospira, Lactobacillus, Parabacteroides, Peptoniphilus, Mediterraneibacter, Clostridium hiranonis, Clostridium hylemonae, Clostridium scindens, Ruminococcus gnavus, Coprococcus comes, Faecalibacterium prausnitzii, Eubacterium ventriosum, Bacteroides pectinophilus, Dorea formicigenerans, Eggerthela lenta, Parabacteroides merdae, Bacteroides dorei, Bacteroides vulgatus, and a combination thereof. In an aspect, the bacterial strains, species, or isolates include bacteria from one or more genus of Bacteroides, Parabacteroides, Butyricimonas, Odoribacter, Porphyromonas, Alistipes, or a combination thereof. In an aspect, the bacterial strains, species, or isolates include bacteria from one or more of Bacteroides uniformis, Bacteroides finegoldii, Bacteroides fragilis, Bacteroides dorei, Bacteroides vulgatus, Bacteroides caccae, Bacteroides ovatus, Bacteroides eggerthii, Bacteroides thetaiotaomicron, Parabacteroides goldsteinii, Parabacteroides distasonis, Parabacteroides merdae, Butyricimonas synergistica, Odoribacter laneus, Porphyromonas somerae, Alistipes indistinctus, Alistipes onderdonkii, Alistipes finegoldii, or a combination thereof.

In an aspect, a method of selecting a patient or a patient population that can benefit from a treatment described herein comprises subjecting a consortium of fecal bacteria of an individual to metagenomic sequencing, screening the metagenomic sequence for the presence of a bile acid metabolism consensus sequence associated with the transformation of a primary bile acid to a secondary bile acid or conversion between secondary bile acids, and selecting the individual as the subject on the basis of a determination that the consensus sequence is absent in the consortium of fecal bacteria or exists in a number of clades in the consortium that is below a threshold number (e.g., less than twenty bacterial clades, less than fifteen bacterial clades, less than ten bacterial clades, or less than five bacterial clades). In an aspect, an individual is selected for treatment with a composition described herein on the basis that no more than twenty, no more than nineteen, no more than eighteen, no more than seventeen, no more than sixteen, no more than fifteen, no more than fourteen, no more than thirteen, no more than twelve, no more than eleven, no more than ten, no more than nine no more than eight, no more than seven, no more than six, no more than five, no more than four, no more than three, no more than two, or no more than one bacterial clade in a fecal microbiota of the subject harbor a bai gene operon or one or more bai genes. In an aspect, a subject is selected for treatment with a composition described herein on the basis that no bacterial clades/no bacteria in a consortium of fecal bacteria of the subject harbor a bai gene operon or one or more bai genes.

In a different aspect, an individual can be selected by comparing the sequence identity of 16S rDNA sequences of known bile acid transforming bacteria to the 16S rDNA sequences identified from a consortium of fecal bacteria from a stool of the individual using metagenomics sequencing. A sequence identity of 100% or near 100% between a 16S rDNA sequence of a clade of bacteria of the consortium and the 16S rDNA sequence of a particular reference bile acid transforming bacteria (e.g., primary bile acid transforming or secondary bile acid converting bacteria) can be used a basis to determine that the individual's consortium of fecal bacteria comprises the respective type of bile acid transforming bacteria. In an aspect, an individual can be selected as a candidate to benefit from a treatment described herein on the basis of determining that a consortium of fecal bacteria from the individual does not comprise any clades having a 16S rDNA sequence with 100% sequence identity, at least 99.5% sequence identity, at least 99% sequence identity, at least 98% identity, at least 97% identity, at least 96% sequence identity or at least 95% sequence identity to a 16S rDNA sequence of a species known to comprise primary bile acid transforming bacteria or secondary bile acid converting bacteria. In another aspect, an individual can be selected as a candidate to benefit from a treatment described herein on the basis of determining that a consortium of fecal bacteria from the individual comprises no more than two clades, no more than three clades, no more than four clades, no more than five clades, no more than six clades, no more than seven clades, no more than eight clades, no more than nine clades, or no more than ten clades having a 16S rDNA sequence with 100% sequence identity, at least 99.5% sequence identity, at least 99% sequence identity, at least 98% identity, at least 97% identity, at least 96% sequence identity or at least 95% sequence identity to a 16S rDNA sequence of a strain or species known to comprise primary bile acid transforming bacteria or secondary bile acid converting bacteria.

In an aspect, an individual can be selected as a candidate to benefit from a treatment described herein on the basis of determining that a consortium of fecal bacteria from the individual does not comprise any clades having a sequence with 100% sequence identity, at least 99.5% sequence identity, at least 99% sequence identity, at least 98% identity, at least 97% identity, at least 96% sequence identity, at least 95% sequence identity, at least 94% sequence identity, at least 93% sequence identity, at least 92% sequence identity, at least 91% sequence identity, at least 90% sequence identity, at least 89% sequence identity, at least 88% sequence identity, at least 87% sequence identity, at least 86% sequence identity, at least 85% sequence identity to 1 or more, 2 or more, 3 or more, 5 or more, 10 or more, 20 or more, 30 or more, 40 or more, or 50 or more bile acid metabolism consensus sequences provided in SEQ ID NOs: 1-252. In another aspect, an individual selected as a candidate to benefit from a treatment described herein has no more than two clades, no more than three clades, no more than four clades, no more than five clades, no more than six clades, no more than seven clades, no more than eight clades, no more than nine clades, or no more than ten clades having an amino acid sequence with 100% sequence identity, at least 99.5% sequence identity, at least 99% sequence identity, at least 98% identity, at least 97% identity, at least 96% sequence identity, at least 95%, at least 94% sequence identity, at least 93% sequence identity, at least 92% sequence identity, at least 91% sequence identity, at least 90% sequence identity, at least 89% sequence identity, at least 88% sequence identity, at least 87% sequence identity, at least 86% sequence identity, at least 85% sequence identity to a bile acid metabolism consensus sequences provided in SEQ ID NOs: 1-252.

In another aspect, a bile acid metabolism consensus sequence identified from a consortium of fecal bacteria from a stool of a donor has a sequence identity of 100% or near 100% to one or more amino acid sequences provided in SEQ ID NOs: 1-252. In an aspect, fecal bacteria from a stool of a donor comprises at least 99.5% sequence identity, at least 99% sequence identity, at least 98% identity, at least 97% identity, at least 96% sequence identity, at least 95% sequence identity, at least 94% sequence identity, at least 93% sequence identity, at least 92% sequence identity, at least 91% sequence identity, at least 90% sequence identity, at least 89% sequence identity, at least 88% sequence identity, at least 87% sequence identity, at least 86% sequence identity, at least 85% sequence identity to a bile acid consensus sequence provided in SEQ ID NOs: 1-252. In another aspect, a consortium of fecal bacteria from a stool of a donor has more than two clades, more than three clades, more than four clades, more than five clades, more than six clades, more than seven clades, more than eight clades, more than nine clades, or more than ten clades having an amino acid sequence with 100% sequence identity, at least 99.5% sequence identity, at least 99% sequence identity, at least 98% identity, at least 97% identity, at least 96% sequence identity, at least 95%, at least 94% sequence identity, at least 93% sequence identity, at least 92% sequence identity, at least 91% sequence identity, at least 90,% sequence identity, at least 89% sequence identity, at least 88% sequence identity, at least 87% sequence identity, at least 86% sequence identity, at least 85% sequence identity to a bile acid metabolism consensus sequence provided in SEQ ID NOs: 1-252.

Another example of a method for identifying or selecting a subject or population of subjects who may benefit from a treatment described herein comprises determining a level or ratio of secondary bile acids produced in a functional assay that exposes fecal bacteria of an individual (e.g., in the form of a fecal microbiota, a consortium of fecal bacteria, or a bacterial isolate) to an appropriate metabolic substrate. In an aspect, such functional assay is an ex vivo functional assay that determines primary bile acid metabolism and comprises exposing the fecal bacteria to a primary bile acid substrate for a period of time and determining the rate of metabolism of the primary bile acid into its corresponding secondary bile acid (e.g., by determining a rate of reduction in level of the primary bile acid, a rate of increase in the level of the secondary bile acid, or a rate of change in ratio of the secondary:primary bile acids). In another aspect, the functional assay is an ex vivo functional assay that detects secondary bile acid metabolism and comprises exposing the fecal bacteria to a secondary bile acid substrate for a period of time and determining the rate of metabolism of a corresponding secondary bile acid (e.g., by determining a rate of reduction in a level of the substrate, a rate of increase in the level of the product, or a rate of change in a ratio of the product to substrate). Where the results of a functional assay determine that fecal bacteria of an individual produce a secondary bile acid at a level or rate that is below a threshold value, such individual can be selected as a candidate to potentially benefit from the treatments described herein. A further example of a method for identifying or selecting a subject or population of subjects who may benefit from a treatment described herein comprises determining a level of a primary and/or secondary bile acid in the stool of an individual. Where such quantification shows that one or more primary bile acids are present in the stool at a level or concentration that is higher than a threshold level or concentration, or one or more secondary bile acids are present in the stool at a level or concentration that is lower than a threshold level or concentration, such individual can be selected as a candidate to potentially benefit from the treatments described herein.

A subject who can benefit from treatment with a composition described herein can further be selected (e.g., in addition to or independently of a method of selection that employs a metagenomics comparison) on the basis of determining that a level of one or more secondary bile acids in a stool of the subject (or a ratio of one or more secondary bile acids to one or more primary bile acids in a stool of a subject) are below a threshold level, or determining that a rate of production of one or more secondary bile acids produced by fecal bacteria of a subject in a functional assay are below a threshold level. For example, raw stool or processed stool can be subjected to a screen or assay to quantify one or more primary and/or secondary bile acids of interest in the stool, and the subject can then be selected for treatment with a composition described herein if the primary bile acid(s) is above a threshold level, the secondary bile acid(s) is below a threshold level, or a ratio of the secondary to primary bile acids is below a threshold level. In another example, fecal bacteria (e.g., in the form of a fecal microbiota, consortium of fecal bacteria, or a bacterial isolate) from the stool of a subject can be subjected to a functional screen, assay or test, for example an “ex vivo” functional screen, assay or test, to determine the capacity of the bacteria to transform a primary bile acid to a corresponding secondary bile acid, or to convert a secondary bile acid to a corresponding secondary bile acid, and the subject can be selected for treatment with a composition described herein if a rate of transformation or conversion of the bile acid substrate to its corresponding secondary bile acid is below a threshold level.

The detection or measurement of primary or secondary bile acids (e.g., directly from stool or in a functional assay) can be by any method. For example, in an aspect, primary or secondary bile acids are detected or measured using chromatography, e.g., thin-layer chromatography (TLC) and/or liquid chromatography (e.g., ultra-performance liquid chromatography), for example in combination with mass spectrometry (e.g., TLC-MS or UPLC-MS. In another aspect, bile acids can be quantified or profiled using metabolomics techniques.

In an aspect, a functional assay comprises exposing fecal bacteria of a subject (e.g., a consortium of fecal bacteria) to a primary bile acid for a period of time (e.g., at least 1 at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24 at least 30, at least 36 hours, at least 42 hours, at least 48, at least 54, at least 60, or greater than 60 hours) and determining the rate of conversion of the primary bile acid to its corresponding bile acid. In an aspect, the primary bile acid is CA and the secondary bile acid is DCA. In another aspect, the primary bile acid is CDCA and the secondary bile acid is LCA. In various aspects, a determination of the rate of conversion or transformation comprises (i) comparing the level of primary bile acid substrate at the beginning and end of the assay, (ii) determining a level of secondary bile acid at the end of the assay, and/or comparing a ratio of the secondary bile acid to the primary bile acid at the beginning and end of the assay. In an aspect, a subject can benefit from treatment or can be selected for treatment on the basis that a ratio of secondary to primary bile acids existing at the end of the functional assay (e.g., during a period of less than 24 hours or less than 12 hours) is less than 1. In another aspect, a subject can benefit from treatment or can be selected for treatment on the basis that a ratio of secondary to primary bile acids existing at the end of the functional assay (e.g., during a period of less than 24 hours or less than 12 hours) is less than 0.5. In another aspect, a subject can benefit from treatment or can be selected for treatment on the basis that a ratio of secondary to primary bile acids existing at the end of the functional assay (e.g., during a period of less than 24 hours or less than 12 hours) is less than 2, less than 1.9, less than 1.8, less than 1.7, less than 1.6, less than 1.5, less than 1.4, less than 1.3, less than 1.2, less than 1.1, less than 1, less than 0.9, less than 0.8, less than 0.7, less than 0.6, less than 0.5, less than 0.4, less than 0.3, less than 0.2 or less than 0.1.

In an aspect, a subject is selected as potentially benefiting from administration with a composition described herein, or for treatment with a composition described herein, based on determining that a level or concentration of at least one secondary bile acid in a stool of the subject or arising from a functional assay (or a ratio of a secondary to primary bile acid in a stool or arising from a functional assay) is lower than a threshold level, wherein the secondary bile acid is selected from the group consisting of deoxycholic acid (DCA), isodeoxycholic acid, glycodeoxycholic acid (GDCA), taurodeoxycholic acid (TDCA), glycolithocholic acid (GLCA), taurolithocholic acid (TLCA), lithocholic acid (LCA), ursodeoxycholic acid (UDCA), isoursodeoxycholic acid, glycoursodeoxycholic acid (GUDCA), tauroursodeoxycholic acid (TUDCA) or a combination thereof.

In an aspect, a subject is selected for treatment or as potentially benefitting from treatment on the basis of a ratio of total secondary bile acids to total primary bile acids in a stool being lower than a threshold value. In an aspect, the subject is selected if the ratio of total secondary bile acids to total primary bile acids is less than 2, less than 1.9, less than 1.8, less than 1.7, less than 1.6, less than 1.5, less than 1.4, less than 1.3, less than 1.2, less than 1.1, less than 1, less than 0.9, less than 0.8, less than 0.7, less than 0.6, less than 0.5, less than 0.4, less than 0.3, less than 0.2 or less than 0.1.

In another aspect, a subject is selected for treatment or as potentially benefitting from treatment if the ratio of a secondary bile acid to its corresponding primary bile acid in a stool of the subject is less than a threshold value. In an aspect, the ratio of secondary bile acid to primary bile acid is selected from the group consisting of DCA:CA, LCA:CDCA, 12-oxoLCA:CA, 3-oxoCA:CA, 7-oxoDCA:CA, 3-oxoLCA:CDCA, 7-oxoLCA:CDCA, and a combination thereof. In an aspect, the subject is selected if the ratio of the secondary bile acid to its corresponding primary bile acid in a stool of the subject is less than 2, less than 1.9, less than 1.8, less than 1.7, less than 1.6, less than 1.5, less than 1.4, less than 1.3, less than 1.2, less than 1.1, less than 1, less than 0.9, less than 0.8, less than 0.7, less than 0.6, less than 0.5, less than 0.4, less than 0.3, less than 0.2 or less than 0.1.

In a further aspect, a subject is selected for treatment or as potentially benefitting from treatment if the ratio of a second secondary bile acid to a first secondary bile acid in a stool of the subject is less than a threshold value. In an aspect, the ratio of second secondary bile acid to first secondary bile acid is selected from the group consisting of IDCA:DCA, GDCA:DCA, TDCA:DCA, GLCA:LCA, TLCA:LCA, UDCA:LCA, isoLCA:3-oxoLCA, isoalloLCA:3-oxoLCA, 3-oxoalloLCA:3-oxoLCA, or a combination thereof. In an aspect, the subject is selected if the ratio of the secondary bile acid to its corresponding primary bile acid in a stool of the subject is less than 2, less than 1.9, less than 1.8, less than 1.7, less than 1.6, less than 1.5, less than 1.4, less than 1.3, less than 1.2, less than 1.1, less than 1, less than 0.9, less than 0.8, less than 0.7, less than 0.6, less than 0.5, less than 0.4, less than 0.3, less than 0.2 or less than 0.1.

In an aspect the at least one secondary bile acid is deoxycholic acid (DCA), and a subject is selected based on a level of DCA (e.g., in a stool of the subject or arising from a bile acid transforming functional assay incorporating fecal bacteria of the subject and a bile acid substrate, e.g., cholic acid) that is lower than a threshold level of DCA, wherein the threshold level is 5 μM, 10 μM, 15 μM, 20 μM, 30 μM, 40 μM, 50 μM, 60 μM, 70 μM, 80 μM, 90 μM, 100 μM, 120 μM, 140 μM, 150 μM, 170 μM, 200 μM, 300 μM, 350 μM, 400 μM, 450 μM, or 500 μM. In another aspect, the threshold level of DCA is between 5 μM and 20 μM, 5 μM and 50 μM, 5 μM and 100 μM, and 5 μM and 150 μM, 5 μM and 200 μM, 5 μM and 800 μM, 10 μM and 20 μM, 10 μM and 50 μM, 10 μM and 100 μM, 10 μM and 150 μM, 10 μM and 200 μM, 50 μM and 100 μM, 50 μM and 200 μM, 100 μM and 200 μM, 200 μM and 400 μM, 200 μM and 600 μM, 400 μM and 800 μM, or 200 μM and 800 μM.

In another aspect, the at least one secondary bile acid is glycodeoxycholic acid (GDCA), and a subject is selected as potentially benefitting from a treatment described herein based on a level of GDCA (e.g., in a stool of the subject or arising from a bile acid transforming functional assay incorporating fecal bacteria of the subject and a bile acid substrate, e.g. DCA) that is lower than a threshold level of GDCA, wherein the threshold level is 5 μM, 10 μM, 15 μM, 20 μM, 30 μM, 40 μM, 50 μM, 60 μM, 70 μM, 80 μM, 90 μM, 100 μM, 120 μM, 140 μM, 150 μM, 170 μM, or 200 μM. In another aspect, the threshold level of GDCA is between 5 μM and 20 μM, 5 μM and 50 μM, 5 μM and 100 μM, and 5 μM and 150 μM, 5 μM and 200 μM, 5 μM and 800 μM, 10 μM and 20 μM, 10 μM and 50 μM, 10 μM and 100 μM, 10 μM and 150 μM, 10 μM and 200 μM, 50 μM and 100 μM, 50 μM and 200 μM, 100 μM and 200 μM, or 200 μM and 800 μM.

In another aspect, the at least one secondary bile acid is taurodeoxycholic acid (TDCA), and a subject is selected as potentially benefitting from a treatment described herein based on a level of TDCA (e.g., in a stool of the subject or arising from a bile acid transforming functional assay incorporating fecal bacteria of the subject and a bile acid substrate, e.g., DCA) that is lower than a threshold level of TDCA, wherein the threshold level is 5 μM, 10 μM, 15 μM, 20 μM, 30 μM, 40 μM, 50 μM, 60 μM, 70 μM, 80 μM, 90 μM, 100 μM, 120 μM, 140 μM, 150 μM, 170 μM, or 200 μM. In another aspect, the threshold level of TDCA is between 5 μM and 20 μM, 5 μM and 50 μM, 5 μM and 100 μM, and 5 μM and 150 μM, 5 μM and 200 μM, 5 μM and 800 μM, 10 μM and 20 μM, 10 μM and 50 μM, 10 μM and 100 μM, 10 μM and 150 μM, 10 μM and 200 μM, 50 μM and 100 μM, 50 μM and 200 μM, 100 μM and 200 μM, or 200 μM and 800 μM.

In another aspect, the at least one secondary bile acid is glycolithocholic acid (GLCA), and a subject is selected as potentially benefitting from a treatment described herein based on a level of GLCA (e.g., in a stool of the subject or arising from a bile acid transforming functional assay incorporating fecal bacteria of the subject and a bile acid substrate, e.g., LCA) that is lower than a threshold level of GLCA, wherein the threshold level is 5 μM, 10 μM, 15 μM, 20 μM, 30 μM, 40 μM, 50 μM, 60 μM, 70 μM, 80 μM, 90 μM, 100 μM, 120 μM, 140 μM, 150 μM, 170 μM, or 200 μM. In another aspect, the threshold level of GLCA is between 5 μM and 20 μM, 5 μM and 50 μM, 5 μM and 100 μM, and 5 μM and 150 μM, 5 μM and 200 μM, 5 μM and 800 μM, 10 μM and 20 μM, 10 μM and 50 μM, 10 μM and 100 μM, 10 μM and 150 μM, 10 μM and 200 μM, 50 μM and 100 μM, 50 μM and 200 μM, 100 μM and 200 μM, or 200 μM and 800 μM.

In another aspect, the at least one secondary bile acid is lithocholic acid (LCA), and a subject is selected as potentially benefitting from a treatment described herein based on a level of LCA (e.g., in a stool of the subject or arising from a bile acid transforming functional assay incorporating fecal bacteria of the subject and a bile acid substrate, e.g., chenodeoxycholic acid) that is lower than a threshold level of LCA, wherein the threshold level is 5 μM, 10 μM, 15 μM, 20 μM, 30 μM, 40 μM, 50 μM, 60 μM, 70 μM, 80 μM, 90 μM, 100 μM, 120 μM, 140 μM, 150 μM, 170 μM, or 200 μM. In another aspect, the threshold level of LCA is between 5 μM and 20 μM, 5 μM and 50 μM, 5 μM and 100 μM, and 5 μM and 150 μM, 5 μM and 200 μM, 5 μM and 800 μM, 10 μM and 20 μM, 10 μM and 50M, 10 μM and 100 μM, 10 μM and 150 μM, 10 μM and 200 μM, 50 μM and 100 μM, 50 μM and 200 μM, 100 μM and 200 μM, or 200 μM and 800 μM. In another aspect, the threshold level of is at least 800 μM, at least 1000 μM, at least 1200 μM, at least 1400 μM, at least 1600 μM, at least 1800 μM, at least 2000 μM, or greater than 2000 μM.

In another aspect, the at least one secondary bile acid is isoalloLCA, and a subject is selected as potentially benefitting from a treatment described herein based on a level of isoalloLCA (e.g., in a stool of the subject or arising from a bile acid transforming functional assay incorporating fecal bacteria of the subject and a bile acid substrate, e.g., chenodeoxycholic acid) that is lower than a threshold level of isoalloLCA, wherein the threshold level is 5 μM, 10 μM, 15 μM, 20 μM, 30 μM, 40 μM, 50 μM, 60 μM, 70 μM, 80 μM, 90 μM, 100 μM, 120 μM, 140 μM, 150 μM, 170 μM, or 200 μM. In another aspect, the threshold level of isoalloLCA is between 5 μM and 20 μM, 5 μM and 50 μM, 5 μM and 100 μM, and 5 μM and 150 μM, 5 μM and 200 μM, 5 μM and 800 μM, 10 μM and 20 μM, 10 μM and 50 μM, 10 μM and 100 μM, 10 μM and 150 μM, 10 μM and 200 μM, 50 μM and 100 μM, 50 μM and 200 μM, 100 μM and 200 μM, or 200 μM and 800 μM. In another aspect, the threshold level of is at least 800 μM, at least 1000 μM, at least 1200 μM, at least 1400 μM, at least 1600 μM, at least 1800 μM, at least 2000 μM, or greater than 2000 μM.

In another aspect, the at least one secondary bile acid is ursodeoxycholic acid (UDCA), and a subject is selected as potentially benefitting from a treatment described herein based on a level of UDCA (e.g., in a stool of the subject or arising from a bile acid transforming functional assay incorporating fecal bacteria of the subject and a bile acid substrate, e.g., LCA) that is lower than a threshold level of UDCA, wherein the threshold level is 5 μM, 10 μM, 15 μM, 20 μM, 30 μM, 40 μM, 50 μM, 60 μM, 70 μM, 80 μM, 90 μM, 100 μM, 120 μM, 140 μM, 150 μM, 170 μM, or 200 μM. In another aspect, the threshold level of UDCA is between 5 μM and 20 μM, 5 μM and 50 μM, 5 μM and 100 μM, and 5 μM and 150 μM, 5 μM and 200 μM, 5 μM and 800 μM, 10 μM and 20 μM, 10 μM and 50 μM, 10 μM and 100 μM, 10 μM and 150 μM, 10 μM and 200 μM, 50 μM and 100 μM, 50 μM and 200 μM, 100 μM and 200 μM, or 200 μM and 800 μM.

In another aspect, the at least one secondary bile acid is glycoursodeoxycholic acid (GUDCA), and a subject is selected as potentially benefitting from a treatment described herein based on a level of GUDCA (e.g., in a stool of the subject or arising from a bile acid transforming functional assay incorporating fecal bacteria of the subject and a bile acid substrate, e.g., UDCA) that is lower than a threshold level of GUDCA, wherein the threshold level is 5 μM, 10 μM, 15 μM, 20 μM, 30 μM, 40 μM, 50 μM, 60 μM, 70 μM, 80 μM, 90 μM, 100 μM, 120 μM, 140 μM, 150 μM, 170 μM, or 200 μM. In another aspect, the threshold level of UDCA is between 5 μM and 20 μM, 5 μM and 50 μM, 5 μM and 100 μM, and 5 μM and 150 μM, 5 μM and 200 μM, 5 μM and 800 μM, 10 μM and 20 μM, 10 μM and 50 μM, 10 μM and 100 μM, 10 μM and 150 μM, 10 μM and 200 μM, 50 μM and 100 μM, 50 μM and 200 μM, 100 μM and 200 μM, or 200 μM and 800 μM.

In another aspect, the at least one secondary bile acid is tauroursodeoxycholic acid (TUDCA), and a subject is selected as potentially benefitting from a treatment described herein based on a level of TUDCA (e.g., in a stool of the subject or arising from a bile acid transforming functional assay incorporating fecal bacteria of the subject and a bile acid substrate, e.g., UDCA) that is lower than a threshold level of TUDCA, wherein the threshold level is 5 μM, 10 μM, 15 μM, 20 μM, 30 μM, 40 μM, 50 μM, 60 μM, 70 μM, 80 μM, 90 μM, 100 μM, 120 μM, 140 μM, 150 μM, 170 μM, or 200 μM. In another aspect, the threshold level of TUDCA is between 5 μM and 20 μM, 5 μM and 50 μM, 5 μM and 100 μM, and 5 μM and 150 μM, 5 μM and 200 μM, 5 μM and 800 μM, 10 μM and 20 μM, 10 μM and 50 μM, 10 μM and 100 μM, 10 μM and 150 μM, 10 μM and 200 μM, 50 μM and 100 μM, 50 μM and 200 μM, 100 μM and 200 μM, or 200 μM and 800 μM.

A subject can further be selected as potentially benefitting from administration of a composition described herein based on a level of one or more primary bile acids in a stool of the subject being above a threshold level. An elevated level of one or more primary bile acids in a stool may be indicative of a deficiency in the ability of fecal bacteria of the subject to utilize the pool of intestinal primary bile acids as a substrate to generate secondary bile acids. In an aspect, a subject is selected on the basis of a level of a primary bile acid that is greater than a threshold level, wherein the primary bile acid is selected from the group consisting of cholic acid (CA) and chenodeoxycholic acid (CDCA) and a combination thereof. In an aspect the threshold level is greater than 1 mM of a primary bile acid. In another aspect, the threshold level is 1 mM, 950 μM, 900 μM, 850 μM, 800 μM, 750 μM, 700 μM, 650 μM, 600 μM, 550 μM, 500 μM, 450 μM, 400 μM, 350 μM, 300 μM, 250 μM, 200 μM, 150 μM, 100 μM, or 50 μM of a primary bile acid.

Disclosed herein is a pharmaceutical composition comprising a bacterial mixture that comprises one or more bacterial isolates that comprise primary bile acid transforming bacteria, secondary bile acid converting bacteria, or a combination thereof. In an aspect, a bacterial mixture comprises at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, or greater than twenty bacterial isolates. In an aspect, administration of a pharmaceutical composition comprising the bacterial mixture to a subject in need thereof rescues a deficiency in bile acid metabolism as a result of the introduction and/or engraftment of one or more of the bacterial isolates/strain or species into the subject's intestinal microbiota and subsequent production of secondary bile acids by the introduced bacterial isolates/strain or species (e.g., by the transformation of primary into secondary bile acids or the conversion of a secondary bile acid to a different secondary bile acid). In aspects, a pharmaceutical composition comprises a plurality of primary bile acid transforming bacterial isolates or a plurality of secondary bile acid converting bacteria to provide for functional redundancy in the composition in the event that a particular bacterial isolate is unable to engraft or engrafts at a reduced efficiency in the intestine of the subject administered the composition.

In an aspect, a bacterial isolate can be selected for inclusion in a pharmaceutical composition described herein based on an ability of such bacterial isolate to correct a deficiency in secondary bile acid metabolism of a subject administered the composition. For example, such bacterial isolate can comprise one or more bai genes, a bai operon or one or more genes capable of mediating a metabolic step in the conversion between secondary bile acids.

In an aspect, a bacterial isolate can be selected for inclusion in a pharmaceutical composition described herein based on the determination that such bacterial isolate corresponds to a bacterial clade having a relative abundance in a patient or population of patients following administration of fecal microbiota transplantation (FMT) therapy that correlates with the rescue of a bile acid metabolic deficiency in the patient or patient population. For example, a bacterial isolate can be selected for inclusion in a composition on the basis that a bacterial clade corresponding to the bacterial isolate comprises one or more bile acid metabolism consensus sequences (e.g., all or a portion of one or more bai genes or a bai operon) and is undetectable in the fecal microbiota of a patient or group of patients prior to administration of an FMT, but present in the fecal microbiota of the patient or group of patients following the FMT. In another example, the bacterial clade can be present in a patient or group of patients prior to an FMT, but increase in relative abundance in the patient or group of patients following the FMT. In an aspect, a bacterial isolate is selected for inclusion in a composition on the basis that a corresponding bacterial clade in a patient following an FMT comprises a bile acid metabolism consensus sequence, and the relative abundance of the corresponding bacterial clade increases in at least one patient, at least two patients, at least three patients, at least four patients, at least five patients, at least six patients, at least seven patients, at least eight patients, at least nine patients, at least ten patients, at least fifteen patients, at least twenty patients, at least twenty five patients, at least thirty patients, at least thirty five patients, at least forty patients, at least forty five patients, at least fifty patients, or more than fifty patients following administration of FMT to treat a disorder in the group of patients. Herein a bacterial isolate “corresponds to” a bacterial clade in a consortium of fecal bacteria (e.g., in the form of a fecal microbiota) where respective 16S rDNA of the bacterial isolate and bacterial clade share at least 95%, at least 96%, at least 97%, at least 98%, at least 99%, at least 99.5%, or 100% sequence identity. The bacterial isolate corresponding to a bacterial clade that rescues the bile acid metabolism deficiency in a patient further comprises the bile acid consensus sequence present in the bacterial clade. Examples of disorders that can be treated with FMT to introduce or increase the relative abundance of a bile acid transforming bacterial clade include C. difficile infection, inflammatory bowel disease, ulcerative colitis, Crohn's disease, an autism spectrum disorder, obesity, chronic fatigue syndrome, hepatic encephalopathy, nonalcoholic steatohepatitis (NASH), a hepatitis B virus infection, primary sclerosing cholangitis (PSC), Parkinson's disease and multiple sclerosis.

In another aspect, a bacterial isolate can be selected for inclusion in a pharmaceutical composition described herein based on a determination that the bacterial isolate corresponds to a bacterial clade that has an increased relative abundance in a population of subjects that do not have or are in remission from a disease or disorder associated with a deficiency in bile acid metabolism. For example, a bacterial isolate comprising bile acid transforming bacteria (e.g., species or strain harboring a bai operon) can be selected on the basis that it corresponds to a bacterial clade in a population of subjects in remission from a disease or disorder but is not present or is present in a reduced relative abundance in an intestinal microbiota of a patient population. A patient or patient population used for the comparison can have any disorder associated with a deficiency in bile acid metabolism, including but not limited to C. difficile infection, inflammatory bowel disease, ulcerative colitis, Crohn's disease, an autism spectrum disorder, obesity, chronic fatigue syndrome, hepatic encephalopathy, nonalcoholic steatohepatitis (NASH), a hepatitis B virus infection, primary sclerosing cholangitis (PSC), Parkinson's disease and multiple sclerosis.

Following the selection of a bacteria isolate on the basis of a genetic correspondence with a bacterial clade (e.g., sharing at least 95%, at least 96%, at least 97%, at least 98%, at least 99%, at least 99.5% or 100% identity of 16SrDNA sequences) determined to be present in a microbiome of a subject or population of subjects at an increased relative abundance following FMT or compared to a patient population having a disorder or condition associated with a deficiency in bile acid metabolism, such bacterial isolate can be directly selected for inclusion in a bacterial mixture or subjected to further analyses, tests or assays that confirm its ability to treat a bile acid metabolism deficiency in a patient. For example, the DNA of the bacterial isolate can be assessed for the existence of bile acid metabolism consensus sequences that mediate or associate with either primary bile acid transformation or secondary bile acid conversion. Alternatively, or in addition, the bacterial isolate can be subjected to one or more “wet” assays such as quantifying the rate of secondary bile acid production (e.g., by exposing the isolate to a substrate comprising either a primary bile acid or a secondary bile acid), either alone or in combination with other strains or species of bacteria. The results of such analyses and assays can be used as a further basis for selecting such bacterial isolate for inclusion in a composition.

In an aspect, a bacterial isolate can be obtained from a laboratory stock or a bacterial cell bank archiving bacterial strain or species originally isolated from a stool of a human donor. For example, a consortium of fecal bacteria can be the source of a bacterial isolate incorporated into a bacterial mixture described herein. In certain aspects, a consortium of fecal bacteria can be cultured on a solid media substrate and one or more bacterial isolates selected as single colonies. In other aspects, a consortium of fecal bacteria can be added to liquid media to produce a mixed bacterial culture that is then serially diluted to produce a culture of the bacterial isolate expanded from a single cell. In aspects, a bacterial isolate can be cultured (e.g., in solid or liquid media) using known techniques. Methods for isolating, purifying, and/or culturing bacterial strain or species are described in Sadowsky et al., WO 2012/122478 and in Borody et al., WO 2012/016287, each of which is incorporated herein by reference.

In certain aspects, a pharmaceutical composition comprises a bacterial isolate capable of transforming a primary bile acid to a secondary bile acid. For example, the bacterial isolate can comprise one or more bai genes or a bai operon. In certain instances, a bacterial composition can comprise one or more primary bile acid transforming bacteria (e.g., bacterial strain) in the form of a bacterial isolate that is selected from the group consisting of Clostridium, Bacillus, Bacteroides, Bifidobacterium, Catenibacterium, Collinsella, Eggerthella, Lachnospira, Lactobacillus, Parabacteroides, Peptoniphilus, Mediterraneibacter, Clostridium hiranonis, Clostridium hylemonae, Clostridium scindens, Ruminococcus gnavus, Coprococcus comes, Faecalibacterium prausnitzii, Eubacterium ventriosum, Bacteroides pectinophilus, Dorea formicigenerans, Eggerthela lenta, Parabacteroides merdae, Bacteroides dorei, Bacteroides vulgatus, and a combination thereof.

In certain aspects, a pharmaceutical composition comprises a bacterial isolate comprising one or more bacteria that is a member of a taxonomical group selected from the group consisting of Bacteroides, Parabacteroides, Butyricimonas, Odoribacter, Porphyromonas, Alistipes, and a combination thereof. In certain aspects, a pharmaceutical composition comprises a bacterial isolate comprising one or more bacteria that is a member of a taxonomical group selected from the group consisting of Bacteroides uniformis, Bacteroides finegoldii, Bacteroides fragilis, Bacteroides dorei, Bacteroides vulgatus, Bacteroides caccae, Bacteroides ovatus, Bacteroides eggerthii, Bacteroides thetaiotaomicron, Parabacteroides goldsteinii, Parabacteroides distasonis, Parabacteroides merdae, Butyricimonas synergistica, Odoribacter laneus, Porphyromonas somerae, Alistipes indistinctus, Alistipes onderdonkii, Alistipes finegoldii, and a combination thereof.

In another aspect of the present disclosure, a bacterial mixture comprises a bacterial isolate comprising one or more bile acid transforming bacteria (either primary bile acid transforming bacteria or secondary bile acid transforming bacteria) that is a member of a taxonomical group selected from the group consisting of Firmicutes, Bacteroidetes, Clostridia, Clostridiales, Clostridiaceae, Lachnospiraceae, Ruminococcaceae, Bacteroidacaeae, Porphyromonadaceae, Prevotellaceae, Eubacterium, Clostridium, Fiaecalibacterium, Roseburia, Butyrivibrio, Blautia, Anaerostipes, Coprococcus, Subdoligranulum, Anaerotruncus, Ruminococcus, Clostridium hiranonis, Clostridium hylemonae, Clostridium scindenrs, Ruminococcus gnavus, Coprococcus comes, Faecalibacterium prausnitzii, Eubacterium ventriosum, Bacteroides pectinophilus, Dorea formicigenerans, Clostridium perfringens, Eubacterium lentum, Eubacterium rectale, Blautia producta, Roseburia intestinalis, Roseburia faecis, Roseburia hominis, Roseburia inulinivorans, Roseburia cecicola, Butyrivibrio fibrisolvens, Eubacterium ramulus, Eubacterium hallii, Eubacterium ruminantium, Eubacterium cylindroides, Eubacterium oxidoreducens, Coprococcus catus, Coprococcus eutactus, Coprococcus comes, Faecalibacterium prausnitzii, Subdoligranulum variabile, Anaerotruncus colihominis, Clostridium nexile, Clostridium hathewayi, Clostridium indolis, Clostridium leptum, Ruminococcus gnavus, Ruminococcus obeum, and Anaerostipes caccae.

In another aspect of the present disclosure, a bacterial mixture comprises a bacterial isolate comprising one or more bacteria that is a member of a taxonomical group selected from the group consisting of Bacteroides, Parabacteroides, Butyricimonas, Odoribacter, Porphyromonas, Alistipes, and a combination thereof. In another aspect of the present disclosure, a bacterial mixture comprises a bacterial isolate comprising one or more bacteria that is a member of a taxonomical group selected from the group consisting of Bacteroides uniformis, Bacteroides finegoldii, Bacteroides fragilis, Bacteroides dorei, Bacteroides vulgatus, Bacteroides caccae, Bacteroides ovatus, Bacteroides eggerthii, Bacteroides thetaiotaomicron, Parabacteroides goldsteinii, Parabacteroides distasonis, Parabacteroides merdae, Butyricimonas synergistica, Odoribacter laneus, Porphyromonas somerae, Alistipes indistinctus, Alistipes onderdonkii, Alistipes finegoldii, and a combination thereof.

As described herein, a bacterial mixture can comprise a consortium of fecal bacteria (e.g., for delivering one or more bile acid transforming bacteria to an intestine of a subject administered the composition). In various aspects, a consortium of fecal bacteria incorporated into a pharmaceutical composition can be cultured or uncultured. In an aspect, a consortium of fecal bacteria harvested from a stool of a donor can be selected for inclusion in a composition on the basis of a level or quantity of a secondary bile acid in a stool of a donor of the fecal bacteria above a threshold value. In another aspect, a consortium of fecal bacteria can be selected for inclusion in a pharmaceutical composition described herein on the basis of the production above a threshold value of a level of a secondary bile acid by the fecal bacteria in an ex vivo assay. In another aspect, a consortium of fecal bacteria can be selected for inclusion in a pharmaceutical composition described herein on the basis of the relative abundance of one or more bile acid-producing bacteria in the consortium, as determined by the presence or abundance of bile acid metabolism consensus sequences. In an aspect, a donor of stool used as starting material to prepare a bacterial mixture is administered a prebiotic prior to donating the stool. In another aspect, a donor of stool used as starting material to prepare bacterial mixture is not administered a prebiotic prior to the donation.

In one aspect, a consortium of fecal bacteria is in the form of an entire or substantially complete fecal microbiota from stool of a donor. In one aspect, a consortium of fecal bacteria comprises an isolated or purified population of live non-pathogenic fecal bacteria. In a further aspect, a consortium of fecal bacteria is in the form of a substantially complete fecal microbiota preparation from a single donor. In another aspect, a pharmaceutical composition described herein comprises a bacterial mixture comprising one or more live, non-pathogenic, bacterial isolates and a consortium of fecal bacteria, for example in the form of uncultured fecal bacteria or a preparation of fermented or co-cultured fecal bacteria.

In an aspect, the processing of a consortium of fecal bacteria involves a treatment selected from the group consisting of ethanol treatment, detergent treatment, heat treatment, irradiation, and sonication. In another aspect, the consortium of fecal bacteria is not processed with an ethanol treatment, detergent treatment, heat treatment, irradiation, or sonication. In one aspect, the a consortium of fecal bacteria is subjected to a separation step selected from the group consisting of density gradients, filtration (e.g., sieves, nylon mesh), and chromatography. In another aspect, the a consortium of fecal bacteria is not subjected to a separation step selected from the group consisting of density gradients, filtration (e.g., sieves, nylon mesh), and chromatography. In another aspect, a pharmaceutical composition administered herein comprises a consortium of fecal bacteria substantially free of donor eukaryotic cells.

In an aspect, a pharmaceutical composition provided or administered herein comprises a consortium of fecal bacteria comprising a Shannon Diversity Index of greater than or equal to 0.3, greater than or equal to 0.4, greater than or equal to 0.5, greater than or equal to 0.6, greater than or equal to 0.7, greater than or equal to 0.8, greater than or equal to 0.9, greater than or equal to 1.0, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2.0, greater than or equal to 2.1, greater than or equal to 2.2, greater than or equal to 2.3, greater than or equal to 2.4, greater than or equal to 2.5, greater than or equal to 3.0, greater than or equal to 3.1, greater than or equal to 3.2, greater than or equal to 3.3, greater than or equal to 3.4, greater than or equal to 3.5, greater than or equal to 3.6, greater than or equal to 3.7, greater than or equal to 3.8, greater than or equal to 3.9, greater than or equal to 4.0, greater than or equal to 4.1, greater than or equal to 4.2, greater than or equal to 4.3, greater than or equal to 4.4, greater than or equal to 4.5, or greater than or equal to 5.0. In another aspect, a pharmaceutical composition comprises fecal microbiota comprising a Shannon Diversity Index of between 0.1 and 3.0, between 0.1 and 2.5, between 0.1 and 2.4, between 0.1 and 2.3, between 0.1 and 2.2, between 0.1 and 2.1, between 0.1 and 2.0, between 0.4 and 2.5, between 0.4 and 3.0, between 0.5 and 5.0, between 0.7 and 5.0, between 0.9 and 5.0, between 1.1 and 5.0, between 1.3 and 5.0, between 1.5 and 5.0, between 1.7 and 5.0, between 1.9 and 5.0, between 2.1 and 5.0, between 2.3 and 5.0, between 2.5 and 5.0, between 2.7 and 5.0, between 2.9 and 5.0, between 3.1 and 5.0, between 3.3 and 5.0, between 3.5 and 5.0, between 3.7 and 5.0, between 31.9 and 5.0, or between 4.1 and 5.0. In one aspect, a Shannon Diversity Index is calculated at the phylum level. In another aspect, a Shannon Diversity Index is calculated at the family level. In one aspect, a Shannon Diversity Index is calculated at the genus level. In another aspect, a Shannon Diversity Index is calculated at the species level. In a further aspect, a pharmaceutical composition comprises a preparation of flora in proportional content that resembles a normal healthy human fecal flora.

In a further aspect, a pharmaceutical composition comprises fecal bacteria (e.g., a consortium of fecal bacteria selected to produce one or more secondary bile acids at or above a threshold level) from at least 1, 2, 3, 4, 5, 6, 7, 8, 9, or 10 different families. In another aspect, a pharmaceutical composition comprises fecal bacteria from at least 11, 12, 13, 14, 15, 16, 17, 18, 19, or 20 different families. In yet another aspect, a pharmaceutical composition comprises fecal bacteria from at least 21, 22, 23, 24, 25, 26, 27, 28, 29, or 30 different families. In a further aspect, a pharmaceutical composition comprises fecal bacteria from at least 31, 32, 33, 34, 35, 36, 37, 38, 39, or 40 different families. In another aspect, a pharmaceutical composition comprises fecal bacteria from at least 41, 42, 43, 44, 45, 46, 47, 48, 49, or 50 different families. In another aspect, a pharmaceutical composition comprises fecal bacteria from between 1 and 10, between 10 and 20, between 20 and 30, between 30 and 40, between 40 and 50 different families. In an aspect, a pharmaceutical composition provided or administered herein comprises a consortium of fecal bacteria comprising no greater than 0.05%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10% weight non-living material/weight biological material. In another aspect, a pharmaceutical composition provided or administered herein comprises a preparation of fecal bacteria comprising no greater than 20%, 25%, 30%, 35%, 40%, 45%, 50%, 55%, 60%, 65%, 70%, 75%, 80%, 85%, 90%, or 95% weight non-living material/weight biological material. In another aspect, a pharmaceutical composition provided or administered herein comprises, consists of, or consists essentially of, particles of non-living stool material and/or particles of biological material of a fecal sample that passes through a sieve, a column, or a similar filtering device having a sieve, exclusion, or particle filter size of 2.0 mm, 1.0 mm, 0.5 mm, 0.33 mm, 0.25 mm, 0.212 mm, 0.180 mm, 0.150 mm, 0.125 mm, 0.106 mm, 0.090 mm, 0.075 mm, 0.063 mm, 0.053 mm, 0.045 mm, 0.038 mm, 0.032 mm, 0.025 mm, 0.020 mm, 0.01 mm, or 0.002 mm. “Non-living stool material” refers to material present in stool when the stool is collected from a donor, and does not include an excipient, e.g., a pharmaceutically inactive substance, such as a cryoprotectant, added during processing of fecal material. “Biological material” refers to the living material in fecal material, and includes microbes including prokaryotic cells, such as bacteria and archaea (e.g., living prokaryotic cells and spores that can sporulate to become living prokaryotic cells), eukaryotic cells such as protozoa and fungi, and viruses. In one aspect, “biological material” refers to the living material, e.g., the microbes, eukaryotic cells, and viruses, which are present in the intestine (e.g., colon) of a normal healthy human. In an aspect, a pharmaceutical composition provided or administered herein comprises an extract of human stool, wherein the composition is substantially odorless. In an aspect, a pharmaceutical composition provided or administered herein comprises fecal material or a fecal floral preparation in a lyophilized, crude, semi-purified or purified formulation.

In an aspect, a consortium of fecal bacteria included in a pharmaceutical composition comprises highly refined or purified fecal flora, e.g., substantially free of non-floral fecal material. In an aspect, a fecal microbiota (comprising a consortium of fecal bacteria) harvested from a donor can be further processed, e.g., to undergo microfiltration before, after, or before and after sieving. In another aspect, a highly purified fecal microbiota product is ultra-filtrated to remove large molecules but retain the therapeutic microflora, e.g., bacteria.

In another aspect, a pharmaceutical composition described herein comprises a purified fecal flora or entire (or substantially entire) microbiota that is (or comprises) an isolate of fecal flora that is at least about 90%, 91%, 92%, 93%, 94%, 95%, 96%, 97%, 98%, 99%, 99.5%, 99.6%, 99.7%, 99.8% or 99.9% isolated or pure, or having no more than about 0.1%, 0.2%, 0.3%, 0.4%, 0.5%, 0.6%, 0.7%, 0.8%, 0.9% or 1.0% or more non-fecal floral material; or, a substantially isolated, purified, or substantially entire microbiota as described in Sadowsky et al., WO 2012/122478 A1, or as described in Borody et al., WO 2012/016287 A2.

In an aspect, a pharmaceutical composition comprises the substantially entire fecal microbiota of stool of a donor. In another aspect, a pharmaceutical composition comprises no antibiotic resistant bacteria. In another aspect, a pharmaceutical composition comprises a preparation of uncultured fecal bacteria that is largely free of extraneous matter (e.g., non-living matter including acellular matter such as residual fiber, DNA, RNA, viral coat material, non-viable material; and living matter such as eukaryotic cells from the donor of the fecal matter).

In an aspect, a consortium of fecal bacteria included in a pharmaceutical composition are derived from a disease-screened stool sample of a human donor. In an aspect, a stool does not include an antibiotic resistant population of bacteria. For example, a composition can comprise a consortium of fecal bacteria or a preparation of viable flora which in proportional content resembles normal healthy human fecal flora which does not include antibiotic resistant populations.

In one aspect, a consortium of fecal bacteria described and used herein comprises one or more, two or more, three or more, four or more, or five or more live fecal bacteria selected from the group consisting of Clostridium hiranonis, Clostridium hylemonae, Clostridium scindens, Ruminococcus gnavus, Coprococcus comes, Faecalibacterium prausnitzii, Eubacterium ventriosum, Bacteroides pectinophilus, Dorea formicigenerans, Acidaminococcus, Akkermansia, Alistipes, Anaerotruncus, Bacteroides, Bifidobacterium, Blautia, Butyrivibrio, Clostridium, Collinsella, Coprococcus, Corynebacterium, Dorea, Enterococcus, Escherichia, Eubacterium, Faecalibacterium, Haemophilus, Holdemania, Lactobacillus, Moraxella, Parabacteroides, Prevotella, Propionibacterium, Raoultella, Roseburia, Ruminococcus, Staphylococcus, Streptococcus, Subdoligranulum, and Veillonella. In one aspect, a consortium of fecal bacteria comprises one or more, two or more, three or more, four or more, or five or more live fecal microorganisms selected from the group consisting of Bacteroides fragilis ssp. vulgatus, Collinsella aerofaciens, Bacteroides fragilis ssp. thetaiotaomicron, Peptostreptococcus productus II, Parabacteroides distasonis, Faecalibacterium prausnitzii, Coprococcus eutactus, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale, Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Coprococcus catus, Aerostipes hadrus, Eubacterium cylindroides, Eubacterium ruminantium, Staphylococcus epidermidis, Eubacterium limosum, Tissirella praeacuta, Fusobacterium mortiferum I, Fusohacterium naviforme, Clostridium innocuum, Clostridium ramosum, Propionibacterium acnes, Ruminococcus flavefaciens, Bacteroides fragilis ssp. ovatus, Fusobacterium nucleatum, Fusobacterium mortiferum, Escherichia coli, Gemella morbillorum, Finegoldia magnus, Streptococcus intermedius, Ruminococcus lactaris, Eubacterium tetme, Eubacterium ramulus, Bacteroides clostridiiformis ssp. clostridliformis, Bacteroides coagulans, Prevotella oralis, Prevotella ruminicola, Odoribacter splanchnicus, and Desuifomonas pigra. In one aspect, a consortium of fecal bacteria comprises one or more, two or more, three or more, four or more, or five or more live fecal microorganisms selected from the group consisting of Bacteroides, Parabacteroides, Butyricimonas, Odoribacter, Porphyromonas, Alistipes, and a combination thereof. In one aspect, a consortium of fecal bacteria comprises one or more, two or more, three or more, four or more, or five or more live fecal microorganisms selected from the group consisting of Bacteroides uniformis, Bacteroides finegoldii, Bacteroides fragilis, Bacteroides dorei, Bacteroides vulgatus, Bacteroides caccae, Bacteroides ovatus, Bacteroides eggerthii, Bacteroides thetaiotaomicron, Parabacteroides goldsteinii, Parabacteroides distasonis, Parabacteroides merdae, Butyricimonas synergistica, Odoribacter laneus, Porphyromonas somerae, Alistipes indistinctus, Alistipes onderdonkii, Alistipes finegoldii, and a combination thereof.

In one aspect, a consortium of fecal bacteria described herein comprises one or more primary bile acid transforming bacterial strain or species. In aspects, a consortium of fecal bacteria includes at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 45, at least 50, or greater than 50 primary bile acid transforming bacterial strain or species.

In one aspect, a consortium of fecal bacteria described herein comprises one or more secondary bile acid converting bacteria. In aspects, a consortium of fecal bacteria includes at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 45, at least 50, or greater than 50 secondary bile acid converting bacterial species.

In an aspect, a pharmaceutical composition can comprise a consortium of fecal bacteria selected on the basis of the presence of one or more bile acid transforming bacteria in the consortium (or in stool from which the consortium is derived), or on the basis of a relative abundance of one or more bile acid transforming bacteria in the consortium (or in stool from which the consortium is derived) at or above a threshold relative abundance. In various aspects, a pharmaceutical composition comprises a consortium of fecal bacteria comprising one or more bile acid transforming bacterial strain or species at a relative abundance in the preparation of at least 0.5, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 45, at least 50, at least 55, at least 60, at least 65, at least 70, at least 75, at least 80, or greater than 80%.

In one aspect, a consortium of fecal bacteria described and used herein lacks or is substantially devoid of one or more, two or more, three or more, four or more, or five or more live fecal microorganisms selected from the group consisting of Acidaminococcus, Akkermansia, Alistipes, Anaerotruncus, Bacteroides, Bifidobacterium, Blautia, Butyrivibrio, Clostridium, Collinsella, Coprococcus, Corynebacterium, Dorea, Farterococcus, Escherichia, Eubacterium, Faecalibacterium, Haemophilus, Holdemania, Lactobacillus, Moraxella, Parabacteroides, Prevotella, Propionibacterium, Raoultella, Roseburia, Ruminococcus, Staphylococcus, Streptococcus, Subdoligranulum, and Veillonella. In one aspect, a consortium of fecal bacteria lacks or is substantially devoid of one or more, two or more, three or more, four or more, or five or live more fecal microorganisms selected from the group consisting of Bacteroides fragilis ssp. vulgatus, Collinsella aerofaciens, Bacteroides fragilis ssp. thetaiotaomicron, Peptostreptococcus productus II, Parabacteroides distasonis, Faecalibacterium prausnitzii, Coprococcus eutactus, Peptostreptococcus productus I, Ruminococcus bromii, Bifidobacterium adolescentis, Gemmiger formicilis, Bifidobacterium longum, Eubacterium siraeum, Ruminococcus torques, Eubacterium rectale, Eubacterium eligens, Bacteroides eggerthii, Clostridium leptum, Bacteroides fragilis ssp. A, Eubacterium biforme, Bifidobacterium infantis, Eubacterium rectale, Coprococcus comes, Pseudoflavonifractor capillosus, Ruminococcus albus, Dorea formicigenerans, Eubacterium hallii, Eubacterium ventriosum I, Fusobacterium russi, Ruminococcus obeum, Eubacterium rectale, Clostridium ramosum, Lactobacillus leichmannii, Ruminococcus callidus, Butyrivibrio crossotus, Acidaminococcus fermentans, Eubacterium ventriosum, Bacteroides fragilis ssp. fragilis, Coprococcus catus, Aerostipes hadrus, Eubacterium cylindroides, Eubacterium ruminantium, Staphylococcus epidermidis, Eubacterium limosum, Tissirella praeacuta, Fusobacterium mortiferum I, Fusobacterium naviforme, Clostridium innocuum, Clostridium ramosum, Propionibacterium acnes, Ruminococcus flavefaciens, Bacteroides fragilis ssp. ovatus, Fusobacterium nucleatum, Fusobacterium mortiferum, Escherichia coli, Gemella morbillorum, Finegoldia magnus, Streptococcus intermedius, Ruminococcus lactaris, Eubacterium tenue, Eubacterium ramulus, Bacteroides clostridiiformis ssp. clostridliformis, Bacteroides coagulans, Prevotella oralis, Prevotella ruminicola, Odoribacter splanchnicus, and Desuifomonas pigra.

In an aspect, a consortium of fecal bacteria for incorporation into a pharmaceutical composition comprise non-pathogenic spores of one or more, two or more, three or more, or four or more Clostridium species selected from the group consisting of Clostridium absonum, Clostridium argentinense, Clostridium baratii, Clostridium botulinum, Clostridium cadaveris, Clostridium carnis, Clostridium celatum, Clostridium chauvoei, Clostridium clostridioforme, Clostridium cochlearium, Clostridium fallax, Clostridium felsineum, Clostridium ghonii, Clostridium glycolicum, Clostridium haemolyticum, Clostridium hastiforme, Clostridium histolyticuum, Clostridium indolis, Clostridium irregulare, Clostridium limosum, Clostridium malenominatum, Clostridium novyi, Clostridium oroticum, Clostridium paraputrificum, Clostridium perfringens, Clostridium piiforme, Clostridium putrefaciens, Clostridium putrificum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and Clostridium villosum. In an aspect, a pharmaceutical composition comprises one or more, two or more, three or more, or four or more non-pathogenic Bacteroides species selected from the group of Bacteroides coprocola, Bacteroides plebeius, Bacteroides massiliensis, Bacteroides vulgatus, Bacteroides helcogenes, Bacteroides pyogenes, Bacteroides tectus, Bacteroides uniformis, Bacteroides stercoris, Bacteroides eggerthii, Bacteroides finegoldii, Bacteroides thetaiotaomicron, Bacteroides ovatus, Bacteroides acidifaciens, Bacteroides caccae, Bacteroides nordii, Bacteroides salyersiae, Bacteroides fragilis, Bacteroides intestinalis, Bacteroides coprosuis, Bacteroides distasonis, Bacteroides goldsteinii, Bacteroides merdae, Bacteroides forsythus, Bacteroides splanchnicus, Bacteroides capillosus, Bacteroides cellulosolvens, and Bacteroides ureolyticus.

In an aspect, a consortium of fecal bacteria for incorporation into a pharmaceutical composition comprise non-pathogenic spores of one or more, two or more, three or more, or four or more bacterial species from a genus selected from the group consisting of Bacteroides, Parabacteroides, Butyricimonas, Odoribacter, Porphyromonas, Alistipes, and a combination thereof. In an aspect, a consortium of fecal bacteria for incorporation into a pharmaceutical composition comprise non-pathogenic spores of one or more, two or more, three or more, or four or more bacterial species selected from the group consisting of Bacteroides uniformis, Bacteroides finegoldii, Bacteroides fragilis, Bacteroides dorei, Bacteroides vulgatus, Bacteroides caccae, Bacteroides ovatus, Bacteroides eggerthii, Bacteroides thetaiotaomicron, Parabacteroides goldsteinii, Parabacteroides distasonis, Parabacteroides merdae, Butvricimonas synergistica, Odoribacter laneus, Porphyromonas somerae, Alistipes indistinctus, Alistipes onderdonkii, Alistipes finegoldii, and a combination thereof.

In an aspect, a consortium of fecal bacteria extracted from a stool of a donor comprises all (100%) of the bacterial strain or species originally present in the stool of the donor. In an aspect, a consortium of fecal bacteria extracted from a stool of a donor comprises 99.9% of the bacterial strain or species originally present in the stool of the donor. In an aspect, a consortium of fecal bacteria extracted from stool of a donor comprises 99.8, 99.7, 99.6, 99.5, 99.4, 99.3, 99.2, 99.1, 99, 98, 97, 96, 95, 94, 93, 92, 91, 90, 89, 88, 87, 85, 84, 83, 82, 81, 80, 75, 70, 65, 60, 55, 50, 45, or 40% of the bacterial strain or species originally present in the stool of the donor.

In an aspect, a pharmaceutical composition disclosed herein comprises a sterile fecal filtrate or a non-cellular fecal filtrate. In one aspect, a sterile fecal filtrate originates from a donor stool. In another aspect, a sterile fecal filtrate originates from cultured microorganisms. In another aspect, a sterile fecal filtrate comprises a non-cellular non-particulate fecal component. In one aspect, a sterile fecal filtrate is made as described in WO2014/078911, published May 30, 2014. In another aspect, a sterile fecal filtrate is made as described in Ott et al., Gastroenterology 152:799-911(2017).

In one aspect, a fecal filtrate comprises secreted, excreted or otherwise liquid components or a microbiota, e.g., biologically active molecules (BAMs), which can be antibiotics or anti-inflammatories, are preserved, retained or reconstituted in a flora extract.

In one aspect, preparation of a fecal filtrate comprises receiving stool from a donor, homogenizing and centrifuging the stool, and then filtering with very high-level filtration using e.g., either metal sieving or Millipore filters, or equivalent, to ultimately permit only cells of bacterial origin to remain, e.g., often less than about 5 micrometers diameter. After the initial centrifugation, the solid material can be separated from the liquid, and the solid ban then be filtered in progressively reducing size filters and tangential filters, e.g., using a Millipore filtration, and optionally, also comprising use of nano-membrane filtering. The filtering can also be done by sieves as described in WO 2012/122478, but in contrast using sieves that are smaller than 0.0120 mm, down to about 0.0110 mm, which ultimately result in having only bacterial cells present.

The supernatant separated during centrifugation can in some aspects be filtered progressively in a filtering, e.g., a Millipore filtering or equivalent systems, to produce a liquid which is finely filtered through an about 0.22 micron filter. This removes all particulate matter including all living matter, including bacteria and viruses. The product then is sterile, but the aim is to remove the bacteria but to keep their secretions, especially antimicrobial bacteriocins, bacteria-derived cytokine-like products and all accompanying Biologically Active Molecules (BAMs), including: thuricin (which is secreted by bacilli in donor stools), bacteriocins (including colicin, troudulixine or putaindicine, or microcin or subtilosin A), lanbiotics (including nisin, subtilin, epidermin, mutacin, mersacidin, actagardine, cinnamycin), lacticins and other antimicrobial or anti-inflammatory compounds.

In one aspect, a pharmaceutical composition comprises reconstituted fecal flora consisting essentially of a combination of a purified fecal microbiota (e.g., a consortium of fecal bacteria) and a non-cellular fecal filtrate. In another aspect, a pharmaceutical composition comprises a purified fecal microbiota (e.g., a consortium of fecal bacteria) supplemented with one or more non-cellular non-particulate fecal components. In one aspect, a pharmaceutical composition comprises one or more non-cellular non-particulate fecal components. In one aspect, one or more non-cellular non-particulate fecal components comprise synthetic molecules, biologically active molecules produced by a fecal microorganism, or both. In another aspect, one or more non-cellular non-particulate fecal components comprise biologically active proteins or peptides, micronutrients, fats, sugars, small carbohydrates, trace elements, mineral salts, ash, mucous, amino acids, nutrients, vitamins, minerals, or any combination thereof. In one aspect, one or more non-cellular non-particulate fecal components comprise one or more biologically active molecules selected from the group consisting of bacteriocin, lanbiotic, and lacticin. In another aspect, one or more non-cellular non-particulate fecal components comprise one or more bacteriocins selected from the group consisting of colicin, troudulixine, putaindicine, microcin, and subtilosin A. In one aspect, one or more non-cellular non-particulate fecal components comprise one or more lanbiotics selected from the group consisting of thuricin, nisin, subtilin, epidermin, mutacin, mersacidin, actagardine, and cinnamycin. In another aspect, one or more non-cellular non-particulate fecal components comprise an anti-spore compound, an antimicrobial compound, an anti-inflammatory compound, or any combination thereof. In a further aspect, one or more non-cellular non-particulate fecal components comprise an interleukin, a cytokine, a leukotriene, an eicosanoid, or any combination thereof.

In another aspect, a pharmaceutical composition comprises both a consortium of fecal bacteria, e.g., a partial or a complete representation of the human gastrointestinal (GI) microbiota, and an isolated, processed, filtered, concentrated, reconstituted and/or artificial liquid component (e.g., fecal filtrate) of the flora (the microbiota) which comprises, among others ingredients, bacterial secretory products such as e.g., bacteriocins (proteinaceous toxins produced by bacteria, including colicin, troudulixine or putaindicine, or microcin or subtilosin A), lanbiotics (a class of peptide antibiotics that contain a characteristic polycyclic thioether amino acid lanthionine or methyllanthionine, and unsaturated amino acids dehydroalanine and 2-aminoisobutyric acid; which include thuricin (which is secreted by bacilli in donor stools), nisin, subtilin, epidermin, mutacin, mersacidin, actagardine, cinnamycin), a lacticin (a family of pore-forming peptidic toxins) and other antimicrobial or anti-inflammatory compounds and/or additional biologically active molecules (BAMs) produced by bacteria or other microorganisms of the microbiota, and/or which are found in the “liquid component” of a microbiota.

In one aspect, a pharmaceutical composition comprising a consortium of fecal bacteria (e.g., selected to comprise one or more bile acid transforming bacteria) is used concurrently with a fecal non-cellular filtrate-based pharmaceutical composition. In another aspect, a patient is treated with a first fecal non-cellular filtrate-based pharmaceutical composition before being given a second pharmaceutical composition comprising a consortium of fecal bacteria (e.g., selected to produce one or more secondary bile acids), or vice versa. In a further aspect, a treatment method comprises three steps: first, antibiotic pretreatment to non-selectively remove infectious pathogen(s); second, a fecal non-cellular filtrate-based treatment step to further suppress selected infectious pathogen(s); and third, treatment with a pharmaceutical composition comprising a consortium of fecal bacteria to re-establish a functional intestinal microbiome.

In an aspect, a composition comprising a consortium of fecal bacteria and/or one or more bacterial isolates administered to a subject having or at risk for a disorder (e.g., a disorder that can be treated by delivery of one or more bile acid transforming bacteria to the intestine of the subject) effects a cure, reduction of the symptoms, or a percentage reduction of symptoms of the disorder based on replacement of bacterial cells endogenous to the intestinal flora of the subject with bacterial cells from the administered bacterial mixture. The change of flora can be as “near-complete” as possible. Typically, the change in enteric flora comprises introduction of an array of flora derived from the stool of a healthy human donor and/or one or more bacterial isolates into the gastro-intestinal system of the subject, which can substantially or completely displace pathogenic enteric flora in a patient requiring such treatment (e.g., an IBD or hepatic encephalopathy patient). In an aspect, engraftment of the administered bacteria in the intestine of the patient results in an enhancement or facilitation of bile acid metabolism by the engrafted bacteria, which as described herein can produce therapeutic effects in the patient.

The pharmaceutical compositions described herein can comprise microbes, e.g., bacteria, derived from a stool of a donor, e.g., a healthy human donor. In an aspect, a composition incorporates a consortium of fecal bacteria derived from all or a portion of a fecal microbiota of a stool of a healthy human donor. For example, a composition can incorporate a substantially complete fecal microbiota of a stool of a healthy human donor. In an aspect, a composition incorporates a bacterial isolate of a fecal microbiota, wherein the bacterial isolate has been purified and/or cultured from the fecal microbiota of a stool from a healthy human donor. The harvesting, extraction and/or purification of a fecal microbiota from a stool can thus be performed to prepare a composition comprising at least one of a consortium of fecal bacteria or a bacterial isolate.

In one aspect, an exemplary fecal microbiota for use in preparing a composition described herein (e.g., comprising a consortium of fecal bacteria or a bacterial isolate) comprises starting material from a human donor. In another aspect, an exemplary fecal microbiota comprises material from one or more healthy human donors. In yet another aspect, an exemplary fecal microbiota comprises starting material from a pool of known, defined donors. In another aspect, a donor is an adult male. In a further aspect, a donor is an adult female. In yet another aspect, a donor is an adolescent male. In another aspect, a donor is an adolescent female. In another aspect, a donor is a female toddler. In another aspect, a donor is a male toddler. In another aspect, a donor is healthy. In one aspect, a human donor is a child below about 18, 15, 12, 10, 8, 6, 4, 3, 2, or 1-year-old. In another aspect, a human donor is an elderly individual. In a further aspect, a human donor is an individual above about 30, 35, 40, 45, 50, 55, 60, 65, 70, 75, 80, 85, 90, or 95 years old. In another aspect, a donor is between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a donor is a young old individual (65-74 years). In one aspect, a donor is a middle old individual (75-84 years). In one aspect, a donor is an old individual (>85 years). In yet another aspect, a donor is a carefully screened, healthy, neurotypical human.

In an aspect, a fecal donor can be prescreened for its fecal microbiome profile. In another aspect, a stool donor can be selected on the basis of the presence or absence of one or more bacterial taxa (e.g., phylum, class, order, family, genus, species, or strain) in the donor's stool, for example that are known to comprise bile acid transforming bacteria. In another aspect, a stool donor can be selected on the basis of the presence or absence of one or more bacterial species. In another aspect, a stool donor can be selected on the basis of the presence of one or more bacterial taxa (e.g., phylum, class, order, family, genus, species, or strain) in the donor's stool at a level above a threshold relative abundance. In another aspect, a stool donor can be selected on the basis of the presence or absence of one or more bacterial species in the donor's stool at a level above a threshold relative abundance. In an aspect, a stool donor can be selected on the basis of the presence or increased relative abundance (compared to the stool of one or more other eligible donors) of one or more bile acid transforming bacteria in the stool of the donor. In another aspect, a stool donor can be selected on the basis of the presence or relative abundance of one or more bacterial taxa (e.g., phylum, class, order, family, genus, species, or strain) of bile acid transforming bacteria.

In an aspect, a relative abundance of one or more bacterial species in stool of a donor can be increased by ingestion by the donor of a prebiotic and/or probiotic that promotes the proliferation or presence of bile acid transforming bacteria, compared to a relative abundance of the bacterial species in the absence of ingesting the probiotic and/or prebiotic. In an aspect, a fecal donor can ingest a stimulant that promotes proliferation of one or more bile acid transforming bacteria prior to donation of a stool for use in preparing a consortium of fecal bacteria for incorporation into a pharmaceutical composition. Examples of stimulants for bile acid transforming bacteria include probiotics and prebiotics. In an aspect, a fecal donor can ingest a probiotic (e.g., comprising one or more bacterial isolates) that comprises one or more bile acid transforming bacteria prior to donation of a stool for use in preparing a consortium of fecal bacteria for incorporation into a pharmaceutical composition. For example, a probiotic administered to a donor can comprise one or more bacterial species of a genus selected from the group consisting of Clostridium, Bacillus, Bacteroides, Bifidobacterium, Catenibacterium, Collinsella, Eggerthella, Lachnospira, Lactobacillus, Parabacteroides, Peptoniphilus, Mediterraneibacter, Clostridium hiranonis, Clostridium hylemonae, Clostridium scindens, Ruminococcus gnavus, Coprococcus comes, Faecalibacterium prausnitzii, Eubacterium ventriosum, Bacteroides pectinophilus, Dorea formicigenerans, Eggerthela lenta, Parabacteroides merdae, Bacteroides dorei, Bacteroides vulgatus, and a combination thereof. In an aspect, a probiotic administered to a donor can comprise one or more bacterial species of a genus selected from the group consisting of Bacteroides, Parabacteroides, Butyricimonas, Odoribacter, Porphyromonas, Alistipes, and a combination thereof. In an aspect, a probiotic administered to a donor can comprise one or more bacterial species selected from the group consisting of Bacteroides uniformis, Bacteroides finegoldii, Bacteroides fragilis, Bacteroides dorei, Bacteroides vulgatus, Bacteroides caccae, Bacteroides ovatus, Bacteroides eggerthii, Bacteroides thetaiotaomicron, Parabacteroides goldsteinii, Parabacteroides distasonis, Parabacteroides merdae, Butyricimonas synergistica, Odoribacter laneus, Porphyromonas somerae, Alistipes indistinctus, Alistipes onderdonkii, Alistipes finegoldii, and a combination thereof. In another aspect, a donor can ingest a prebiotic prior to donation of a stool for use in preparing a consortium of fecal bacteria for incorporation into a pharmaceutical composition. In an aspect, administration of a prebiotic to a donor increases a relative abundance of one or more bile acid transforming bacteria in the stool of the donor. In an aspect, a prebiotic administered to a donor prior to collection of stool from the donor comprises, for example, an amino acid (e.g., valine, leucine, isoleucine), lactic acid, ammonium nitrate, amylose, barley mulch, biotin, carbonate, cellulose, chitin, choline, fructooligosaccharides (FOSs), fructose, glucose, glycerol, heteropolysaccharide, histidine, homopolysaccharide, hydroxyapatite, inulin, isomaltulose, lactose, lactulose, maltodextrins, maltose, nitrogen, oligodextrose, oligofructose, oligofructose-enriched inulin, an oligosaccharide (e.g. comprising a galactooligosaccharide (GOS), trans-galactooligosaccharide, fructooligosaccharide (FOS), xylooligosaccharides (XOS), mannooligosaccharide, or chitooligosaccharide), pectin, phosphate salts, phosphorus, polydextroses, polyols, potash, potassium, sodium nitrate, starch, sucrose, sulfur, sun fiber, tagatose, thiamine, trehalose, vitamins, a water-soluble carbohydrate, a fermentable polysaccharide, a dietary fiber, resistant starch, barley, white navy bean powder, or a combination thereof.

In an aspect, a carefully screened donor undergoes a complete medical history and physical exam. Donors are excluded if they have a risk of infectious agents. Additional exclusion criteria can comprise the following:
Known viral infection with Hepatitis B, C or HIV.
Known exposure to HIV or viral hepatitis at any time
High risk behaviors including sex for drugs or money, men who have sex with men, more than one sexual partner in the preceding 12 months, any past use of intravenous drugs or intranasal cocaine, history of incarceration.
Tattoo or body piercing within 12 months.
Travel to areas of the world where risk of traveler's diarrhea is higher than the US.
Current communicable disease, e.g., upper respiratory viral infection.
History of irritable bowel syndrome. Specific symptoms can include frequent abdominal cramps, excessive gas, bloating, abdominal distension, fecal urgency, diarrhea, constipation.
History of inflammatory bowel disease such as Crohn's disease, ulcerative colitis, microscopic colitis.
Chronic diarrhea.
Chronic constipation or use of laxatives.
History of gastrointestinal malignancy or known colon polyposis.
History of any abdominal surgery, e.g., gastric bypass, intestinal resection, appendectomy, cholecystectomy, etc.
Use of Probiotics or any other over the counter aids used by the potential donor for purpose of regulating digestion. Yogurt and kefir products are allowed if taken merely as food rather than nutritional supplements.
Antibiotics for any indication within the preceding 6 months.
Any prescribed immunosuppressive or anti-neoplastic medications.
Metabolic Syndrome, established or emerging. Criteria used for definition here are stricter than any established criteria. These include history of increased blood pressure, history of diabetes or glucose intolerance.
Known systemic autoimmunity, e.g., connective tissue disease, multiple sclerosis.
Known atopic diseases including asthma or eczema.
Chronic pain syndromes including fibromyalgia, chronic fatigue syndrome.
Ongoing (even if intermittent) use of any prescribed medications, including inhalers or topical creams and ointments.
Neurologic, neurodevelopmental, and neurodegenerative disorders including autism, Parkinson's disease.
General. Body mass index >26 kg/m2, central obesity defined by waste:hip ratio >0.85 (male) and >0.80 (female).
Blood pressure >135 mmHg systolic and >85 mmHg diastolic.
Skin—presence of a rash, tattoos or body piercing placed within a year, or jaundice
Enlarged lymph nodes.
Wheezing on auscultation.
Hepatomegaly or stigmata of liver disease.
Swollen or tender joints. Muscle weakness.
Abnormal neurologic examination.
Stool positive for Clostridium difficile toxin B tested by PCR.
Stool positive for any coronavirus tested by PCR.
Stool positive for COVID-19 tested by PCR.
Positive stool cultures for any of the routine pathogens including Salmonella, Shigella, Yersinia, Campylobacter, E. coli 0157:H7.
Abnormal ova and parasites examination.
Positive Giardia, Cryptosporidium, or Helicobacter pylori antigens
Positive screening for any viral illnesses, including HIV 1 and 2, Viral Hepatitis A IgM, Hepatitis surface antigen and core Ab.
Abnormal RPR (screen for syphilis).
Any abnormal liver function tests including alkaline phosphatase, aspartate aminotransaminase, alanine aminotransferase.
Raised serum triglycerides >150 mg/Dl.
HDL cholesterol <40 mg/dL (males) and <50 mg/dL (females).
High sensitivity CRP >2.4 mg/.
Raised fasting plasma glucose (>100 mg/dL).

In one aspect, provided herein is a process for collecting and processing a stool to give rise to a processed or purified consortium of fecal bacteria. The process can comprise first collecting a stool or portion thereof from one or more healthy (e.g., screened) donor(s). In one aspect, a fresh stool or portion thereof is transported via a stool collection device, which can provide or comprises a suitably oxygen free (or substantially oxygen free) appropriate container. In one aspect, the container can be made oxygen free by e.g., incorporating into the container a built in or clipped-on oxygen-scavenging mechanism, e.g., oxygen scavenging pellets as described e.g., in U.S. Pat. No. 7,541,091. In another aspect, the container itself is made of an oxygen scavenging material, e.g., oxygen scavenging iron, e.g., as described by O2BLOCK™, or equivalents, which uses a purified and modified layered clay as a performance-enhancing carrier of oxygen-scavenging iron, the active iron is dispersed directly in the polymer. In one aspect, oxygen-scavenging polymers are used to make the container itself or to coat the container, or as pellets to be added; e.g., as described in U.S. Pat. App. Pub. 20110045222, describing polymer blends having one or more unsaturated olefinic homopolymers or copolymers; one or more polyamide homopolymers or copolymers; one or more polyethylene terephthalate homopolymers or copolymers; that exhibit oxygen-scavenging activity. In one aspect, oxygen-scavenging polymers are used to make the container itself or to coat the container, or as pellets to be added; e.g., as described in U.S. Pat. App. Pub. 20110008554, describing compositions comprising a polyester, a copolyester ether and an oxidation catalyst, wherein the copolyester ether comprises a polyether segment comprising poly(tetramethylene-co-alkylene ether). In one aspect, oxygen-scavenging polymers are used to make the container itself or to coat the container, or as pellets to be added; e.g., as described in U.S. Pat. App. Pub. 201000255231, describing a dispersed iron/salt particle in a polymer matrix, and an oxygen scavenging film with oxygen scavenging particulates.

Alternatively, in addition to or in place of the oxygen-scavenging mechanism, the air in the container can be removed to produce a vacuum or replaced (completely or substantially) with nitrogen and/or other inert non-reactive gas or gases. In one aspect, the container simulates (creates) partially, substantially or completely an anaerobic environment.

In one aspect, the stool (e.g., fecal sample) is held in an aesthetically acceptable container that will not leak or release an odor yet maintain an anaerobic environment internally. In one aspect, the container is sterile before receiving the stool comprising the fecal flora.

In one aspect, a stool provided herein is maintained at room temperature during most or all of its transportation to and/or storage at a storage facility, e.g., a “stool bank”. For example, once delivered to a processing stool bank, the stool can be stored at ambient temperature, e.g., room temperature. In one aspect, stabilizing agents, such as a cryoprotectant such as glycerol or trehalose or an antioxidant such as cysteine, are added to the harvested and/or stored fecal material.

In one aspect, the stool is tested for various pathogens, as noted above. In one aspect, once cleared of infective agents, a stool or portion or sample thereof is homogenized in a liquid (e.g., comprising one or more of a buffer, saline, a cryoprotectant or an antioxidant) and filtered to remove large particles of matter. In one aspect, the stool is subdivided into desired volumes, e.g., which can be between 5 cc and 3 or more liters. For example, in one aspect, a container comprises a 50 gram (g) stool, which can be held in an appropriate oxygen resistant plastic, e.g., a metallized polyethylene terephthalate polyester film, or a metallized MYLAR™.

In one aspect, the stool is subject to homogenization by for example, mixing, agitating, stirring, shaking or grinding. In certain aspects, a stool sample is diluted with a homogenization buffer prior to homogenization. A homogenization buffer can, for example, contain a cryoprotectant (e.g., trehalose or glycerol), an antioxidant or reducing agent (e.g., cysteine), and saline and/or a buffer (e.g., 0.25×PBS at pH 7.4).

In one aspect, to separate the non-bacterial components from the fecal microbiota, the stool can be homogenized and filtered from rough particulate matter and/or fiber. In one aspect, the microscopic fiber/nonliving matter is then separated from the bacteria. Several methods can be used, including e.g., recurrent filtration with filter sizes, e.g., progressively coming down to the size of a typical bacterium. In one aspect, different filters are used to isolate bacterial sp., or a technique as used by Williams in WO 2011/033310A1, which uses a crude technique of filtration with a gauze.

In one aspect, a filtration procedure for filtering whole stool is suitably used to reach the highest concentration of almost 100% bacteria in a filtered composition. In one aspect, the filtering procedure is a two-step procedure suitably using glass fiber depth filters for initial clarification. In one aspect, the stool is filtered under positive pressure. In one aspect, this would be using a combination or sandwich configuration with a 30 micron PVDF filter. In one aspect, this sandwich procedure will be filtering the product under positive pressure. Later, membrane concentration can, in one aspect, be used as another step to reduce the volume of the filtrate. In one aspect, this can be done prior to freeze drying or spray drying under nitrogen cover.

Alternative membranes that can be used for filtration include, but not limited to, nylon filters, cellulose nitrate filters, polyethersulfone (PES) filters, polytetrafluorethylene (PTFE) filters, TEFLON™ filters, mixed cellulose Ester filters, polycarbonate filters, polypropylene filters, Polyvinylchloride (PVC) filters or quartz filters. Various combinations of these can be used to achieve a high purity of bacteria with solids and liquid removed.

In another aspect, a pharmaceutical composition comprises a bacterial mixture comprising a consortium of fecal bacteria supplemented, spiked, enriched, or enhanced with one or more bacterial isolates (e.g., a probiotic or a bacterial isolate selected from a consortium of fecal bacteria on the basis that such bacterial isolate corresponds to a bacterial clade associated with alleviation of a deficiency in bile acid metabolism in a patient). For example, a bacterial mixture can comprise a consortium of fecal bacteria spiked with one or more bacterial isolates comprising one or more bile acid transforming bacteria. By enriching or spiking a consortium of fecal bacteria derived from a stool (e.g., in the form of a fecal microbiota) of a healthy donor with one or more non-pathogenic bacterial isolates (e.g. comprising one or more bile acid transforming bacteria), a bacterial mixture can be produced in which the amount of a particular bacterial species (i.e. the spiked-in bacterial isolate(s)) can be accounted for and precisely controlled. Without wishing to be bound by theory, this can be advantageous, for example, where a bacterial isolate mixed with the consortium of fecal bacteria is capable of alleviating or treating a disorder of a subject (e.g., by rescuing a deficiency in bile acid metabolism), but insufficient or less effective when administered on its own (apart from the co-administration of a consortium of fecal bacteria) to generate a complete or maximal or optimal treatment response in the subject. Co-administration to a subject of one or more bacterial isolates together with a consortium of fecal bacteria (i.e., derived from a healthy donor) provides the subject with the advantage of the bile acid transforming activity of the administered bacterial isolate combined with multi-factorial benefits conferred by the “microbiota scaffold” component of the mixture. These co-administered species present in the consortium of fecal bacteria may combine to, for example, provide for the necessary milieux or matrix (e.g., via one or more factors released or secreted by the bacterial species) to enable cells of the bacterial isolate to engraft in the intestine and/or induce an optimal response in the subject, or may directly induce a response in the subject that combines and/or synergizes with a response induced by the bacterial isolate to treat the subject. Accordingly, in certain aspects, a pharmaceutical composition comprising a mixture of one or more bacterial isolates and consortium of fecal bacteria can be more effective in treating a disorder of a subject (e.g., a disorder that can be treated by intestinal delivery of one or more bile acid transforming bacteria) than a composition comprising either the bacterial isolate or the consortium of fecal bacteria alone.

### Pharmaceutical Compositions, Formulations, and Administration

Described herein are pharmaceutical compositions comprising a bacterial mixture in various formulations. Any pharmaceutical composition (and/or additional therapeutic agents) described herein can take the form of tablets, pills, pellets, capsules, capsules containing liquids, capsules containing multiparticulates, powders, solutions, emulsion, drops, suppositories, emulsions, aerosols, sprays, suspensions, delayed-release formulations, sustained-release formulations, controlled-release formulations, or any other form suitable for use.

The formulations comprising the pharmaceutical compositions described herein can conveniently be presented in unit dosage forms. For example, the dosage forms can be prepared by methods which include the step of bringing the therapeutic agents into association with a carrier, which constitutes one or more accessory ingredients. For example, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by press tableting).

In another aspect, a pharmaceutical composition can include a pharmaceutically acceptable carrier. As used herein, a “pharmaceutically acceptable carrier” refers to a non-toxic solvent, dispersant, excipient, adjuvant, or other material which is mixed with a live bacterium in order to permit the formation of a pharmaceutical composition, e.g., a dosage form capable of administration to the patient. A pharmaceutically acceptable carrier can be liquid (e.g., saline), gel or solid form of diluents, adjuvant, excipients or an acid resistant encapsulated ingredient. Suitable diluents and excipients include pharmaceutical grades of physiological saline, dextrose, glycerol, mannitol, lactose, starch, magnesium stearate, sodium saccharin, cellulose, magnesium carbonate, and the like, and a combination thereof. In another aspect, a pharmaceutical composition can contain auxiliary substances such as wetting or emulsifying agents, stabilizing or pH buffering agents. In an aspect, a pharmaceutical composition contains about 1%-5%, 5%-10%, 10%-15%, 15-20%, 20%-25%, 25-30%, 30-35%, 40-45%, 50%-55%, 1%-95%, 2%-95%, 5%-95%, 10%-95%, 15%-95%, 20%-95%, 25%-95%, 30%-95%, 35%-95%, 40%-95%, 45%-95%, 50%-95%, 55%-95%, 60%-95%, 65%-95%, 70%-95%, 45%-95%, 80%-95%, or 85%-95% of active ingredient. In an aspect, a pharmaceutical composition contains about 2%-70%, 5%-60%, 10%-50%, 15%-40%, 20%-30%, 25%-60%, 30%-60%, or 35%-60% of active ingredient.

In an aspect, a pharmaceutical composition can include or be incorporated into tablets, drenches, boluses, capsules or premixes. Formulation of these active ingredients into such dosage forms can be accomplished by means of methods well known in the pharmaceutical formulation arts. See, e.g., U.S. Pat. No. 4,394,377. Filling gelatin capsules with any desired form of the active ingredients readily produces capsules. If desired, these materials can be diluted with an inert powdered diluent, such as sugar, starch, powdered milk, purified crystalline cellulose, or the like to increase the volume for convenience of filling capsules.

In an aspect, for preparing solid compositions such as tablets, an active ingredient is mixed with a pharmaceutical carrier, e.g., conventional tableting ingredients such as cornstarch, lactose, sucrose, sorbitol, talc, stearic acid, magnesium stearate, dicalcium phosphate or gums, or other pharmaceutical diluents, e.g. water, to form a solid preformulation composition containing a homogeneous mixture of a composition described herein. When referring to these preformulation compositions as homogeneous, it is meant that the active ingredient is dispersed evenly throughout the composition so that the composition can be readily subdivided into equally effective unit dosage forms such as tablets, pills and capsules. This solid preformulation composition is then subdivided into unit dosage forms of the type described above containing a desired amount of an active ingredient (e.g., at least about 10⁵, 10⁶, 10⁷, 10⁸, 10⁹, 10¹⁰, 10¹¹, 10¹², or 10¹³ CFUs). A pharmaceutical composition described herein can be flavored.

In an aspect, a pharmaceutical composition comprising a bacterial mixture described herein (and optionally one or more additional therapeutic agents) is formulated as a composition adapted for a mode of administration described herein.

In various aspects, the administration of the pharmaceutical compositions is any one of oral, intravenous, intraperitoneal, and parenteral. For example, routes of administration include, but are not limited to, oral, intraperitoneal, intravenous, intramuscular, or rectal. In various aspects, the administration of the pharmaceutical compositions is oral, naso-gastric, antegrade gastrointestinal, retrograde gastrointestinal, endoscopic, or enema.

In an aspect, a pharmaceutical composition described herein can be formulated as a composition adapted for oral administration. Compositions for oral delivery can be in the form of tablets, lozenges, aqueous or oily suspensions, granules, powders, sprinkles, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration to provide sustained delivery of the bacterial mixture over an extended period of time. Selectively permeable membranes surrounding an osmotically active agent are also suitable for orally administered compositions. In these latter platforms, fluid from the environment surrounding the capsule is imbibed by a driving compound, which swells to displace the agent or agent composition through an aperture. These delivery platforms can provide an essentially zero order delivery profile as opposed to the spiked profiles of immediate release formulations. A time-delay material, such as glycerol monostearate or glycerol stearate, can also be useful. Oral compositions can include standard excipients such as mannitol, lactose, starch, magnesium stearate, sodium saccharin, cellulose, ethacrylic acid and derivative polymers thereof, and magnesium carbonate. In an aspect, the excipients are of pharmaceutical grade. Suspensions, in addition to the active compounds, can contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

In various aspects, a pharmaceutical composition is formulated as a solid dosage form such as a tablets, dispersible powder, granule, or capsule. In an aspect, the pharmaceutical composition is formulated as a capsule. In another aspect, the pharmaceutical composition is formulated as a tablet. In yet another aspect, the pharmaceutical composition is formulated as a soft-gel capsule. In a further aspect, the pharmaceutical composition is formulated as a gelatin capsule.

In an aspect, a pharmaceutical composition is in the form of an enema composition which can be reconstituted with an appropriate diluent; an enteric-coated capsule; an enteric-coated microcapsule; an acid-resistant tablet; an acid-resistant capsules; an acid-resistant microcapsule; powder for reconstitution with an appropriate diluent for naso-enteric infusion or colonoscopic infusion; powder for reconstitution with appropriate diluent, flavoring and gastric acid suppression agent for oral ingestion; powder for reconstitution with food or drink; or food or food supplement comprising enteric-coated and/or acid-resistant microcapsules of the composition, powder, jelly, or liquid.

In various aspects, formulations can additionally comprise a pharmaceutically acceptable carrier or excipient. As one skilled in the art will recognize, the formulations can be in any suitable form appropriate for the desired use and route of administration.

In some dosage forms, a pharmaceutical composition described herein is mixed with at least one inert, pharmaceutically acceptable excipient or carrier such as sodium citrate, dicalcium phosphate, etc., and/or a) fillers or extenders such as starches, lactose, sucrose, glucose, mannitol, silicic acid, microcrystalline cellulose, and Bakers Special Sugar, etc., b) binders such as, for example, carboxymethylcellulose, alginates, gelatin, polyvinylpyrrolidone, sucrose, acacia, polyvinyl alcohol, polyvinylpyrrolidone, methylcellulose, hydroxypropyl cellulose (HPC), and hydroxymethyl cellulose etc., c) humectants such as glycerol, etc., d) disintegrating agents such as agar-agar, calcium carbonate, potato or tapioca starch, alginic acid, certain silicates, sodium carbonate, cross-linked polymers such as crospovidone (cross-linked polyvinylpyrrolidone), croscarmellose sodium (cross-linked sodium carboxymethylcellulose), sodium starch glycolate, etc., e) solution retarding agents such as paraffin, etc., f) absorption accelerators such as quaternary ammonium compounds, etc., g) wetting agents such as, for example, cetyl alcohol and glycerol monostearate, etc., h) absorbents such as kaolin and bentonite clay, etc., and i) lubricants such as talc, calcium stearate, magnesium stearate, solid polyethylene glycols, sodium lauryl sulfate, glyceryl behenate, etc., and mixtures of such excipients. One of skill in the art will recognize that particular excipients can have two or more functions in the oral dosage form. In the case of an oral dosage form, for example, a capsule or a tablet, the dosage form can also comprise buffering agents.

In an aspect, a pharmaceutical composition comprising a bacterial mixture is combined with one or more pharmaceutically acceptable cryoprotectants, lyoprotectants, binders, disintegrants, excipients, fillers, and/or preservatives, acid suppressants, antacids, H2 antagonists, and proton pump inhibitors, or combinations thereof.

In an aspect, a pharmaceutical composition comprising a bacterial mixture is combined with other adjuvants such as antacids to dampen bacterial inactivation in the stomach. (e.g., Mylanta, Mucaine, Gastrogel). In another aspect, acid secretion in the stomach could also be pharmacologically suppressed using H2-antagonists or proton pump inhibitors. An example H2-antagonist is ranitidine. An example proton pump inhibitor is omeprazole. In one aspect, an acid suppressant is administered prior to administering, or in co-administration with, a pharmaceutical composition.

In one aspect, a pharmaceutical composition administered herein further comprises an acid suppressant, an antacid, an H2 antagonist, a proton pump inhibitor or a combination thereof. In one aspect, a pharmaceutical composition administered herein is substantially free of non-living matter. In another aspect, a pharmaceutical composition administered herein substantially free of acellular material selected from the group consisting of residual fiber, DNA, viral coat material, and non-viable material. In another aspect, a pharmaceutical composition administered does not comprise an acid suppressant, an antacid, an H2 antagonist, a proton pump inhibitor or a combination thereof. In yet another aspect, a pharmaceutical composition administered does not comprise an acid suppressant. In another aspect, a pharmaceutical composition administered does not comprise an antacid. In another aspect, a pharmaceutical composition administered does not comprise an H2 antagonist. In another aspect, a pharmaceutical composition administered does not comprise a proton pump inhibitor. In another aspect, a pharmaceutical composition administered does not comprise metoclopramide.

In an aspect, a bacterial mixture is dry or powdered, e.g., when it includes lyophilized bacterial cells/spores or comprises dry binders, fillers, and dispersants. Alternately, the bacterial mixture can be aqueous, e.g., when it comprises non-dry binders, fillers, and dispersants.

In an aspect, a bacterial mixture described herein can be subject to lyophilization or spray drying. As used herein, “lyophilization” or “freeze drying” refers to the process of drying a material by first freezing it and then encouraging the ice within it to sublimate in a vacuum environment.

In one aspect, a bacterial mixture comprises a lyophilized or spray dried formulation further comprising a reducing agent and/or antioxidant. In certain aspects, the reducing agent comprises cysteine selected from the group consisting of D-cysteine and L-cysteine. In another aspect, cysteine is at a concentration of at least about 0.025%. In one aspect, cysteine is at a concentration of about 0.025%. In another aspect, cysteine is at a concentration of 0.025%. In another aspect, another reducing agent other than cysteine is used in lieu of, or in combination with cysteine. In an aspect, another reducing agent is selected from the group comprising ascorbic acid, sodium ascorbate, thioglycolic acid, sodium sulfite, sodium bisulfite, sodium metabisulfite, potassium metabisulfite, glutathione, methionine, thioglycerol, and alpha tocopherol.

In one aspect, cysteine is at a concentration of at least about 0.005%, at least about 0.01%, at least about 0.015%, at least about 0.02%, at least about 0.025%, at least about 0.03%, at least about 0.035%, at least about 0.04%, at least about 0.045%, at least about 0.05%, at least about 0.055%, at least about 0.06%, at least about 0.065%, at least about 0.07%, at least about 0.075%, at least about 0.08%, at least about 0.085%, at least about 0.09%, at least about 0.095%, at least about 0.1%, at least about 0.12%, at least about 0.14%, at least about 0.16%, at least about 0.18%, at least about 0.2%, at least about 0.25%, at least about 0.3%, at least about 0.4%, at least about 0.5%, at least about 0.6%, at least about 0.7%, at least about 0.8%, at least about 0.9%, at least about 1%, at least about 2%, at least about 4%, at least about 6%, at least about 8%, at least about 10%, at least about 12%, at least about 14%, at least about 16%, at least about 18%, at least about 20%, at least about 22%, at least about 24%, or at least about 26%.

In one aspect, a bacterial mixture comprises a cryoprotectant or mixture of cryoprotectants. As used herein, a “cryoprotectant” refers to a substance that is added to a formulation in order to protect an active ingredient during freezing. For example, a cryoprotectant can comprise, consist essentially of, or consist of polyethylene glycol, skim milk, erythritol, arabitol, sorbitol, glucose, fructose, alanine, glycine, proline, sucrose, lactose, ribose, trehalose, dimethyl sulfoxide (DMSO) or equivalent, a glycerol, a polyethylene glycol (PEG) or equivalent, or an amino acid (e.g., alanine, glycine, proline). In an aspect of the present disclosure, a cryoprotectant can be selected from the group comprising 5% Sucrose; 10% Sucrose; 10% Skim milk; 10% Trehalose with 2.5% sucrose; 5% Trehalose with 2.5% sucrose; 5% Mannitol; 5% Mannitol with 0.1% Polysorbate 80; 10% Mannitol; 10% Mannitol with 0.1% Polysorbate 80; 5% Trehalose; 5% Trehalose with 0.1% Polysorbate 80; 10% Trehalose; and 10% Trehalose with 0.1% Polysorbate 80.

In an aspect, a bacterial mixture comprises a lyoprotectant. As used herein, a “lyoprotectant” refers to a substance that is added to a formulation in order to protect an active ingredient during lyophilization. In one aspect, the same substance or the same substance combination is used as both a cryoprotectant and a lyoprotectant. Exemplary lyoprotectants include sugars such as sucrose or trehalose; an amino acid such as monosodium glutamate or histidine; a methylamine such as betaine; a lyotropic salt such as magnesium sulfate; a polyol such as trihydric or higher sugar alcohols, e.g. glycerin, erythritol, glycerol, arabitol, xylitol, sorbitol, and mannitol; propylene glycol; polyethylene glycol; Pluronics; and a combination thereof. In an aspect, a lyoprotectant is a non-reducing sugar, such as trehalose or sucrose. In an aspect, a cryoprotectant or a lyoprotectant consists essentially of, or consists of, one or more substances mentioned in this paragraph and the paragraph above.

In an aspect, a cryoprotectant or a lyoprotectant comprise an intracellular agent, e.g., DMSO, Glycerol, or PEG, which penetrates inside the cell preventing the formation of ice crystals that could result in membrane rupture. In an aspect, a cryoprotectant or a lyoprotectant comprise an extracellular agent, e.g., sucrose, trehalose, or dextrose, which does not penetrate into the cell membrane but acts to improve the osmotic imbalance that occurs during freezing.

In an aspect, a bacterial mixture incorporated into a pharmaceutical composition is in the form of a powder. In an aspect, a powdered formulation of bacteria is produced by lyophilizing or spray drying a liquid formulation comprising the bacteria and a saline or buffer.

In one aspect, the present disclosure provides a pharmaceutical composition comprising a lyophilized fecal microbe preparation comprising a lyophilization formulation comprising at least about 12.5% trehalose.

In an aspect, a lyophilized formulation comprises trehalose. In an aspect, a lyophilized formulation comprises 2% to 30%, 3% to 25%, 4% to 20%, 5% to 15%, 6% to 10%, 2% to 30%, 2% to 25%, 2% to 20%, 2% to 15%, or 2% to 10% trehalose. In an aspect, a lyophilized formulation comprises at least 2%, 3%, 4%, 5%, 6%, 7%, 8%, 9%, 10%, or 15% trehalose. In an aspect, a lyophilized formulation comprises at most 2%, 3%, 4%, 5%, 6%, 7%, 8%, 9%, 10%, or 15% trehalose. In another aspect, a lyophilized formulation comprises about 5% trehalose. In another aspect, a lyophilized formulation comprises trehalose and sucrose. In another aspect, a lyophilized formulation comprises between about 8% and 12% trehalose with between about 1.5% and 3.5% sucrose and between about 0.5% and 1.5% NaCl.

In one aspect, a lyophilization formulation comprises at least about 5%, at least about 7.5%, at least about 10%, at least about 12.5%, at least about 13%, at least about 13.5%, at least about 14%, at least about 14.5%, at least about 15%, at least about 15.5%, at least about 16%, at least about 16.5%, at least about 17%, at least about 17.5%, at least about 18%, at least about 18.5%, at least about 19%, at least about 19.5%, at least about 20%, at least about 22.5%, at least about 25%, at least about 27.5%, at least about 30%, at least about 32.5%, at least about 35%, at least about 37.5%, at least about 40%, at least about 42.5%, at least about 45%, at least about 47.5%, at least about 50%, at least about 52.5%, at least about 55%, at least about 57.5%, or at least about 60% of trehalose.

In an aspect, a pharmaceutical composition provided herein, after at least 12 weeks of storage at ambient temperature or lower, is effective for treating or preventing a disorder in a patient by delivering one or more bile acid transforming bacteria to an intestine of the patient. In an aspect, a pharmaceutical composition remains effective after at least 4, 8, 10, 16, 20, 24, 30, 40, 50, 60, 70, 80 or 100 weeks of storage at ambient temperature or lower.

In an aspect, a bacterial mixture can be prepared by spray drying a liquid formulation comprising fecal bacteria, for example a consortium of fecal bacteria or a bacterial isolate. In an aspect, a consortium of fecal bacteria is processed by homogenizing the bacteria in a liquid comprising a buffer and spray drying the homogenized bacteria to form a powder. In an aspect, the liquid formulation is not frozen prior to spray drying.

In an aspect, a pharmaceutical composition described herein can be lyophilized or freeze dried or spray dried and stored at ambient temperatures (e.g., room temperature), at a freezing temperature, or at between about 2° C. and 8° C. In an aspect, freeze-drying allows the majority of cells to remain viable, and produces a powdered form of the product that can be gently pulverized into a powder. The powder, or lyophilized or freeze-dried composition, then can be encapsulated into a carrier, e.g., a tablet, geltab, pill or capsule, e.g., an enteric-coated capsule, or placed into oil-filled capsules for ingestion. Alternatively, the freeze-dried or lyophilized product, or powder, can be reconstituted at ambient temperatures before delivery to an individual in e.g., a fluid, e.g., a sterile fluid, such as saline, a buffer or a media such as a fluid-glucose-cellobiose agar (RGCA) media.

For freeze-drying, in an aspect, bacteria are held in a liquid that will prevent bursting of cells on thawing. This can include various stabilizers, e.g., glycerol and appropriate buffers, and/or ethylene glycol. In an aspect, the cryoprotecting process uses final concentrations of stabilizer(s) of between about 10% and 80%, 20% and 70%, 30% and 60%, or 40% and 50%, depending on the stabilizer(s) used; in an aspect, this helps stabilize proteins by preventing formation of ice crystals that would otherwise destroy protein structures.

In an aspect, stabilizers that help reduce destruction of living bacteria include skim milk, erythritol, arabitol, sorbitol, glucose, fructose and other polyols. Polymers such as dextran and polyethylene glycol can also be used to stabilize bacterial cells.

In an aspect, manufacturing a pharmaceutical composition can comprise steps of: (1) coating the exterior of a dissociated capsule (i.e., comprising separate capsule body and capsule cap) with the exterior enteric coating, (2) filling the capsule body with a bacterial mixture (e.g., comprising a bacterial isolate and/or a consortium of fecal bacteria), and (3) closing the capsule cap over the capsule body, thereby encapsulating the bacterial mixture in the enteric-coated capsule.

Optionally, manufacturing a pharmaceutical composition can comprise steps of: (1) coating the exterior of a dissociated capsule (i.e., comprising separate capsule body and capsule cap) with the exterior enteric coating, (2) coating the interior of the dissociated capsule with an interior coating, (3) filling the capsule body with a bacterial mixture (e.g., comprising a bacterial isolate and/or a consortium of fecal bacteria), and (4) closing the capsule cap over the capsule body, thereby encapsulating the bacterial mixture in the dual-coated capsule.

Alternately, manufacturing a pharmaceutical composition can comprise step of: (1) coating the interior of the dissociated capsule (i.e., comprising separate capsule body and capsule cap) with an interior coating, (2) coating the exterior of a dissociated capsule with the exterior enteric coating, (3) filling the capsule body with a bacterial mixture (e.g., comprising a bacterial isolate and/or a consortium of fecal bacteria), and (4) closing the capsule cap over the capsule body, thereby encapsulating the bacterial mixture in the dual-coated capsule.

In an aspect, one or more additional therapeutic agents can be included in a pharmaceutical composition, and encapsulated by the capsule.

In an aspect, the bodies and caps of gelatin capsules (e.g., size #00) are separated. An exterior enteric coating suspension is prepared by dispersing one or more enteric coating polymers along with other components in a solution. The exterior enteric coating suspension is applied to the exterior of separated capsule bodies and caps, e.g., using a fluid bed Wurster column coater, Fluid Bed Coater, or an equivalent). The capsules are fluidized in the product bowl and the exterior enteric coating suspension is sprayed to produce the outer coating to a target of between about 2 mg/cm2 and 6 mg/cm2, e.g., 3 mg/cm2. After completion of this step, the capsules are set to dry, e.g., between about 8 hours and 24 hours. After drying, exemplary capsules are weighed to calculate weight gain from the exterior enteric coating. Capsules can be inspected for irregularities.

In an aspect, EUDRAGIT® S100 (poly(methacrylic acid, methylmethacrylate)), starch, triethyl citrate, and PlasACRYL™ T20 are dissolved in a solution of water, ethanol, and n-butanol, mixed, and then charged to a suitable spraying device. The solution is then spray coated on the outer surface of the capsule bodies and capsule caps to a target weight gain. The capsule bodies and capsule caps are allowed to dry for about 8 hours to about 24 hours, or longer, e.g., for a week, a month, or more, before further procession, e.g., filling with a bacterial mixture.

In an aspect, it may be desirable to provide an amount of the bacterial mixture to a capsule's cap in addition to providing the composition in the capsule's body. In this aspect, more of the composition will be included in a capsule and/or less air will be contained in a closed capsule.

In an aspect, the interior surface of a capsule comprises an internal coating.

Any of the above-described compositions and materials (e.g., bacterial mixtures, inner coatings, capsules, and outer coatings) can be combined into a pharmaceutical composition described herein. A skilled artisan would know how to select an inner coating; capsule, and outer coating according to his/her present need, which could be based, for example, on a specific bacterial isolate(s) incorporated into a bacterial mixture of the composition and/or the desired delivery location in a subject (e.g., in the colon or small intestine, including the ileum, jejunum or duodenum) of a component of the bacterial mixture (e.g. comprising a bacterial isolate and/or a consortium of fecal bacteria and/or an additional therapeutic agent).

Additional relevant teachings are disclosed in WO 2007122374, which is hereby incorporated herein by reference in its entirety.

In an aspect, during the manufacture of a pharmaceutical composition, a pharmaceutically-acceptable cryoprotectant, lyoprotectant, binder, disintegrant, filler, preservative, acid suppressant, antacid, H2 antagonist, and proton pump inhibitor, or combination thereof can be mixed into the pharmaceutical composition (e.g., comprising a bacterial mixture) to promote desirable properties.

In an aspect, the pharmaceutical composition comprises a surface-active agent. Surface active agents suitable for use include, but are not limited to, any pharmaceutically acceptable, non-toxic surfactant. Classes of surfactants suitable for use include, but are not limited to, polyethoxylated fatty acids, PEG-fatty acid diesters, PEG-fatty acid mono- and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-olyoxypropylene block copolymers, sorbitan fatty acid esters, lower alcohol fatty acid esters, ionic surfactants, and mixtures thereof. In some aspects, compositions can comprise one or more surfactants including, but not limited to, sodium lauryl sulfate, polysorbate 20, polysorbate 40, polysorbate 60, polysorbate 80, and triethyl citrate.

In an aspect, the pharmaceutical composition comprises pharmaceutically acceptable plasticizers to obtain the desired mechanical properties such as flexibility and hardness. Such plasticizers include, but are not limited to, triacetin, citric acid esters, triethyl citrate, phthalic acid esters, dibutyl sebacate, cetyl alcohol, polyethylene glycols, polysorbates or other plasticizers.

In another aspect, the pharmaceutical composition comprises one or more application solvents. Some of the more common solvents that can be used to apply, for example, a delayed-release coating composition include isopropyl alcohol, acetone, methylene chloride and the like.

In yet another aspect, the pharmaceutical composition comprises one or more alkaline materials. Alkaline material suitable for use in compositions include, but are not limited to, sodium, potassium, calcium, magnesium and aluminum salts of acids such as phosphoric acid, carbonic acid, citric acid and other aluminum/magnesium compounds. In addition, the alkaline material can be selected from antacid materials such as aluminum hydroxides, calcium hydroxides, magnesium hydroxides and magnesium oxide.

Besides inert diluents, the orally administered compositions can also include adjuvants such as sweetening, flavoring, and perfuming agents.

In various aspects, the pharmaceutical compositions are formulated for systemic or local delivery. In an aspect, administration is systemic. In another aspect, it may be desirable to administer locally to the area in need of treatment.

Various methods can be used to formulate and/or deliver a pharmaceutical composition (e.g., comprising a bacterial mixture and/or additional therapeutic agent) described herein to a location of interest. For example, the pharmaceutical compositions can be formulated for delivery to the gastrointestinal (GI) tract. The GI tract includes organs of the digestive system such as mouth, esophagus, stomach, small intestine, duodenum, jejunum, ileum, large intestine and rectum and includes all subsections thereof (e.g. the small intestine may include the duodenum, jejunum and ileum; the large intestine may include the colon transversum, colon descendens, colon ascendens, colon sigmoidenum and cecum). For example, the compositions can be formulated for delivery of one or more active agents to one or more of the stomach, small intestine, large intestine and rectum, or any subsection thereof (e.g. duodenum, jejunum and ileum, colon transversum, colon descendens, colon ascendens, colon sigmoidenum and cecum). In some aspects, the compositions described herein can be formulated for delivery of one or more active agents to the upper or lower GI tract. In an aspect, a composition can be administered to a subject, by, for example, directly or indirectly contacting the mucosal tissues of the GI tract with the composition.

In various aspects, the administration of the pharmaceutical compositions is into the GI tract via, for example, oral delivery, nasogastral tube, intestinal intubation (e.g. an enteral tube or feeding tube such as, for example, a jejunal tube or gastro-jejunal tube, etc.), direct infusion (e.g., duodenal infusion), endoscopy, colonoscopy, or enema.

In one aspect, a method comprises administering a pharmaceutical composition orally, by enema, or via rectal suppository. In one aspect, a pharmaceutical composition administered herein is formulated as an enteric coated (and/or acid-resistant) capsule or microcapsule, or formulated as part of or administered together with a food, a food additive, a dairy-based product, a soy-based product or a derivative thereof, a jelly, a gelatin-based chewable (e.g., gummy), flavored liquid, ice block, ice cream, or a yogurt. In another aspect, a pharmaceutical composition administered herein is formulated as an acid-resistant enteric coated capsule. A pharmaceutical composition can be provided as a powder for sale in combination with a food or drink. A food or drink can be a dairy-based product or a soy-based product. In another aspect, a food or food supplement contains enteric-coated and/or acid-resistant microcapsules containing a pharmaceutical composition.

In an aspect, a pharmaceutical composition comprises a liquid culture. In another aspect, a pharmaceutical composition is homogenized, lyophilized, pulverized and powdered. It can then be infused, dissolved such as in saline, as an enema. Alternatively, the powder can be encapsulated as enteric-coated and/or acid-resistant delayed release capsules for oral administration. In an aspect, the powder can be double encapsulated with acid-resistant/delayed release capsules for oral administration. These capsules can take the form of enteric-coated and/or acid-resistant delayed release microcapsules. A powder can be provided in a palatable form for reconstitution for drinking or for reconstitution as a food additive. In a further aspect, a food is yogurt. In one aspect, a powder can be reconstituted to be infused via naso-duodenal infusion.

In another aspect, a pharmaceutical composition administered herein is in a liquid, frozen, freeze-dried, spray-dried, foam-dried, lyophilized, or powder form. In a further aspect, a pharmaceutical composition administered herein is formulated as a delayed or gradual enteric release form. In another aspect, a pharmaceutical composition administered herein comprises an excipient, a saline, a buffer, a buffering agent, or a fluid-glucose-cellobiose agar (RGCA) media. In another aspect, a pharmaceutical composition administered herein comprises a cryoprotectant. In one aspect, a cryoprotectant comprises polyethylene glycol, skim milk, erythritol, arabitol, sorbitol, glucose, fructose, alanine, glycine, proline, sucrose, lactose, ribose, trehalose, dimethyl sulfoxide (DMSO), glycerol, or a combination thereof.

In various aspects, provided herein are modified-release formulations comprising a bacterial mixture (e.g., comprising a bacterial isolate and/or a consortium of fecal bacteria), wherein the formulation releases a substantial amount of the bacterial mixture (and optionally additional therapeutic agents) into one or more regions of the GI tract. For example, the formulation can release at least about 60% of the bacterial isolates after the stomach and into one or more regions of the GI tract.

In various aspects, the modified-release formulation can release at least 60% of the bacterial mixture (and optionally additional therapeutic agents) after the stomach into one or more regions of the intestine. For example, the modified-release formulation can release at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the bacterial mixture (and optionally additional therapeutic agents) in the intestines.

In various aspects, the modified-release formulation can release at least 60% of the bacterial mixture (and optionally additional therapeutic agents) in the small intestine. For example, the modified-release formulation can release at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the bacterial mixture (and optionally additional therapeutic agents) in the small intestine (e.g., one or more of duodenum, jejunum, ileum, and ileocecal junction).

In various aspects, the modified-release formulation can release at least 60% of the bacterial mixture (and optionally additional therapeutic agents) in the large intestine. For example, the modified-release formulation can release at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the bacterial isolates (and/or additional therapeutic agents) in the large intestine (e.g., one or more of cecum, ascending, transverse, descending or sigmoid portions of the colon, and rectum).

In some aspects, the pharmaceutical composition is formulated for release in the stomach. In other aspects, the pharmaceutical composition is formulated so as to not substantially release the bacterial mixture in the stomach.

In certain aspects, the modified-release formulation releases the bacterial mixture (and optionally additional therapeutic agents) at a specific pH. For example, in some aspects, the modified-release formulation is substantially stable in an acidic environment and substantially unstable (e.g., dissolves rapidly or is physically unstable) in a near neutral to alkaline environment. In some aspects, stability is indicative of not substantially releasing while instability is indicative of substantially releasing. For example, in some aspects, the modified-release formulation is substantially stable at a pH of about 7.0 or less, or about 6.5 or less, or about 6.0 or less, or about 5.5 or less, or about 5.0 or less, or about 4.5 or less, or about 4.0 or less, or about 3.5 or less, or about 3.0 or less, or about 2.5 or less, or about 2.0 or less, or about 1.5 or less, or about 1.0 or less. In some aspects, the present formulations are stable in lower pH areas and therefore do not substantially release in, for example, the stomach. In some aspects, modified-release formulation is substantially stable at a pH of about 1 to about 4 or lower and substantially unstable at pH values that are greater. In these aspects, the modified-release formulation does not substantially release in the stomach. In these aspects, the modified-release formulation substantially releases in the small intestine (e.g. one or more of the duodenum, jejunum, and ileum) and/or large intestine (e.g. one or more of the cecum, ascending colon, transverse colon, descending colon, and sigmoid colon). In some aspects, modified-release formulation is substantially stable at a pH of about 4 to about 5 or lower and consequentially is substantially unstable at pH values that are greater and therefore is not substantially released in the stomach and/or small intestine (e.g. one or more of the duodenum, jejunum, and ileum). In these aspects, the modified-release formulation substantially releases in the large intestine (e.g. one or more of the cecum, ascending colon, transverse colon, descending colon, and sigmoid colon). In various aspects, the pH values recited herein can be adjusted as known in the art to account for the state of the subject, e.g. whether in a fasting or postprandial state.

In some aspects, the modified-release formulation is substantially stable in gastric fluid and substantially unstable in intestinal fluid and, accordingly, is substantially released in the small intestine (e.g. one or more of the duodenum, jejunum, and ileum) and/or large intestine (e.g. one or more of the cecum, ascending colon, transverse colon, descending colon, and sigmoid colon).

In some aspects, the modified-release formulation is stable in gastric fluid or stable in acidic environments. These modified-release formulations release about 30% or less by weight of the pharmaceutical composition (e.g., comprising a bacterial mixture) in the modified-release formulation in gastric fluid with a pH of about 4 to about 5 or less, or simulated gastric fluid with a pH of about 4 to about 5 or less, in about 15, or about 30, or about 45, or about 60, or about 90 minutes. Modified-release formulations of can release from about 0% to about 30%, from about 0% to about 25%, from about 0% to about 20%, from about 0% to about 15%, from about 0% to about 10%, about 5% to about 30%, from about 5% to about 25%, from about 5% to about 20%, from about 5% to about 15%, from about 5% to about 10% by weight of the composition in the modified-release formulation in gastric fluid with a pH of 4-5, or less or simulated gastric fluid with a pH of 4-5 or less, in about 15, or about 30, or about 45, or about 60, or about 90 minutes. Modified-release formulations can release about 1%, about 2%, about 3%, about 4%, about 5%, about 6%, about 7%, about 8%, about 9%, or about 10% by weight of the total composition in the modified-release formulation in gastric fluid with a pH of 5 or less, or simulated gastric fluid with a pH of 5 or less, in about 15, or about 30, or about 45, or about 60, or about 90 minutes.

In some aspects, the modified-release formulation is unstable in intestinal fluid. These modified-release formulations release about 70% or more by weight of the bacterial mixture and/or additional therapeutic agent in the modified-release formulation in intestinal fluid or simulated intestinal fluid in about 15, or about 30, or about 45, or about 60, or about 90 minutes. In some aspects, the modified-release formulation is unstable in near neutral to alkaline environments. These modified-release formulations release about 70% or more by weight of the bacterial mixture and/or additional therapeutic agent in the modified-release formulation in intestinal fluid with a pH of about 4-5 or greater, or simulated intestinal fluid with a pH of about 4-5 or greater, in about 15, or about 30, or about 45, or about 60, or about 90 minutes. A modified-release formulation that is unstable in near neutral or alkaline environments can release 70% or more by weight of the pharmaceutical composition (e.g., comprising a microbial cocktail) in the modified-release formulation in a fluid having a pH greater than about 5 (e.g., a fluid having a pH of from about 5 to about 14, from about 6 to about 14, from about 7 to about 14, from about 8 to about 14, from about 9 to about 14, from about 10 to about 14, or from about 11 to about 14) in from about 5 minutes to about 90 minutes, or from about 10 minutes to about 90 minutes, or from about 15 minutes to about 90 minutes, or from about 20 minutes to about 90 minutes, or from about 25 minutes to about 90 minutes, or from about 30 minutes to about 90 minutes, or from about 5 minutes to about 60 minutes, or from about 10 minutes to about 60 minutes, or from about 15 minutes to about 60 minutes, or from about 20 minutes to about 60 minutes, or from about 25 minutes to about 90 minutes, or from about 30 minutes to about 60 minutes.

Examples of simulated gastric fluid and simulated intestinal fluid include, but are not limited to, those disclosed in the 2005 Pharmacopeia 23NF/28USP in Test Solutions at page 2858 and/or other simulated gastric fluids and simulated intestinal fluids known to those of skill in the art, for example, simulated gastric fluid and/or intestinal fluid prepared without enzymes.

In various aspects, the modified-release formulation can be substantially stable in chyme. For example, there is, in some aspects, a loss of less about 50% or about 40%, or about 30%, or about 20%, or about 10% of the activity or viability of the bacteria in the bacterial mixture in about 10, or 9, or 8, or 7, or 6, or 5, or 4, or 3, or 2, or 1 hour from administration.

In various aspects, the modified-release formulations can be designed for immediate release (e.g. upon ingestion). In various aspects, the modified-release formulations can have sustained-release profiles, i.e. slow release of the active ingredient(s) in the body (e.g., GI tract) over an extended period of time. In various aspects, the modified-release formulations can have a delayed-release profile, i.e. not immediately release the active ingredient(s) upon ingestion; rather, postponement of the release of the active ingredient(s) until the composition is lower in the GI tract; for example, for release in the small intestine (e.g., one or more of duodenum, jejunum, ileum) or the large intestine (e.g., one or more of cecum, ascending, transverse, descending or sigmoid portions of the colon, and rectum). For example, a composition can be enteric coated to delay release of the active ingredient(s) until it reaches the small intestine or large intestine.

In various aspects, the modified-release formulations can utilize one or more modified-release coatings such as delayed-release coatings to provide for effective, delayed yet substantial delivery of the bacterial mixture to the GI tract together with, optionally, additional therapeutic agents.

In an aspect, the delayed-release coating includes an enteric agent that is substantially stable in acidic environments and substantially unstable in near neutral to alkaline environments. In an aspect, the delayed-release coating contains an enteric agent that is substantially stable in gastric fluid. The enteric agent can be selected from, for example, solutions or dispersions of methacrylic acid copolymers, cellulose acetate phthalate, hydroxypropylmethyl cellulose phthalate, polyvinyl acetate phthalate, carboxymethylethylcellulose, and EUDRAGIT®-type polymer (poly(methacrylic acid, methylmethacrylate), hydroxypropyl methylcellulose acetate succinate, cellulose acetate trimellitate, shellac or other suitable enteric coating polymers. The EUDRAGIT®-type polymers include, for example, EUDRAGIT® FS 30D, L 30 D-55, L 100-55, L 100, L 12,5, L 12,5 P, RL 30 D, RL PO, RL 100, RL 12,5, RS 30 D, RS PO, RS 100, RS 12,5, NE 30 D, NE 40 D, NM 30 D, S 100, S 12, 5, and S 12,5 P. Similar polymers include Kollicoat® MAE 30 DP and Kollicoat® MAE 100 P. In some aspects, one or more of EUDRAGIT® FS 30D, L 30 D-55, L 100-55, L 100, L 12,5, L 12,5 P RL 30 D, RL PO, RL 100, RL 12,5, RS 30 D, RS PO, RS 100, RS 12,5, NE 30 D, NE 40 D, NM 30 D, S 100, S 12,5 S 12,5 P, Kollicoat® MAE 30 DP and Kollicoat® MAE 100 P is used. In various aspects, the enteric agent can be a combination of the foregoing solutions or dispersions.

In certain aspects, one or more coating system additives are used with the enteric agent. For example, one or more PlasACRYL™ additives can be used as an anti-tacking agent coating additive. Illustrative PlasACRYL™ additives include, but are not limited to, PlasACRYL™ HTP20 and PlasACRYL™ T20.

In another aspect, the delayed-release coating can degrade as a function of time when in aqueous solution without regard to the pH and/or presence of enzymes in the solution. Such a coating can comprise a water insoluble polymer. Its solubility in aqueous solution is therefore independent of the pH. The term “pH independent” as used herein means that the water permeability of the polymer and its ability to release pharmaceutical ingredients is not a function of pH and/or is only very slightly dependent on pH. Such coatings can be used to prepare, for example, sustained release formulations. Suitable water insoluble polymers include pharmaceutically acceptable non-toxic polymers that are substantially insoluble in aqueous media, e.g., water, independent of the pH of the solution. Suitable polymers include, but are not limited to, cellulose ethers, cellulose esters, or cellulose ether-esters, i.e., a cellulose derivative in which some of the hydroxy groups on the cellulose skeleton are substituted with alkyl groups and some are modified with alkanoyl groups. Examples include ethyl cellulose, acetyl cellulose, nitrocellulose, and the like. Other examples of insoluble polymers include, but are not limited to, lacquer, and acrylic and/or methacrylic ester polymers, polymers or copolymers of acrylate or methacrylate having a low quaternary ammonium content, or mixture thereof and the like. Other examples of insoluble polymers include EUDRAGIT RS®, EUDRAGIT RL®, and EUDRAGIT NE®. Insoluble polymers can include polyvinyl esters, polyvinyl acetals, polyacrylic acid esters, butadiene styrene copolymers, and the like. In an aspect, colonic delivery is achieved by use of a slowly eroding wax plug (e.g., various PEGS, including for example, PEG6000).

In a further aspect, the delayed-release coating can be degraded by a microbial enzyme present in the gut flora. In an aspect, the delayed-release coating can be degraded by bacteria present in the small intestine. In another aspect, the delayed-release coating can be degraded by bacteria present in the large intestine.

In various aspects, the modified release formulation can be designed for release in the colon. Various colon-specific delivery approaches can be utilized. For example, the modified release formulation can be formulated using a colon-specific drug delivery system (CODES) as described for example, in Li et al., AAPS PharmSciTech (2002), 3(4). 1-9, the entire contents of which are incorporated herein by reference. Drug release in such a system is triggered by colonic microflora coupled with pH-sensitive polymer coatings. For example, the formulation can be designed as a core tablet with three layers of polymer. The first coating is an acid-soluble polymer (e.g., EUDRAGIT E), the outer coating is enteric, along with a hydroxypropyl methylcellulose barrier layer interposed in between. In another aspect, colon delivery can be achieved by formulating the pharmaceutical composition (e.g., comprising a microbial cocktail) with specific polymers that degrade in the colon such as, for example, pectin. The pectin can be further gelled or crosslinked with a cation such as a zinc cation. In an aspect, the formulation is in the form of ionically crosslinked pectin beads which are further coated with a polymer (e.g., EUDRAGIT polymer). Additional colon specific formulations include, but are not limited to, pressure-controlled drug delivery systems (prepared with, for example, ethylcellulose) and osmotic controlled drug delivery systems (i.e., ORDS-CT).

Formulations for colon specific delivery of the bacterial mixture (and/or additional therapeutic agents), as described herein, can be evaluated using, for example, in vitro dissolution tests. For example, parallel dissolution studies in different buffers can be undertaken to characterize the behavior of the formulations at different pH levels. Alternatively, in vitro enzymatic tests can be carried out. For example, the formulations can be incubated in fermenters containing suitable medium for bacteria, and the amount of drug released at different time intervals is determined. Drug release studies can also be done in buffer medium containing enzymes or rat or guinea pig or rabbit cecal contents and the amount of drug released in a particular time is determined. In a further aspect, in vivo evaluations can be carried out using animal models such as dogs, guinea pigs, rats, and pigs. Further, clinical evaluation of colon specific drug delivery formulations can be evaluated by calculating drug delivery index (DDI) which considers the relative ratio of RCE (relative colonic tissue exposure to the drug) to RSC (relative amount of drug in blood i.e. that is relative systemic exposure to the drug). Higher drug DDI indicates better colon drug delivery. Absorption of drugs from the colon can be monitored by colonoscopy and intubation.

In various aspects, the present formulations provide for substantial uniform delivery of the bacterial mixture (and/or additional therapeutic agent) in the area of release in the GI tract. In an aspect, the present formulations minimize patchy or heterogeneous release of the bacterial mixture.

In various aspects, the present formulations provide for release of multiple doses of one or more bacterial mixtures along the GI tract. For example, the composition and/or formulation can release multiple doses of the same bacterial mixture at different locations along the intestines, at different times, and/or at different pH. Alternatively, the composition and/or formulation can release a dose of different bacterial mixtures at different locations along the intestines, at different times, and/or at a different pH. In an aspect, the pharmaceutical composition comprises a first bacterial mixture comprising one or more bacterial isolates that is released at a first location in the intestine, and a second bacterial mixture comprising a consortium of fecal bacteria that is released at a second location in the intestine. In an aspect, the first bacterial mixture is released in the ileum, and the second bacterial mixture is released in the colon.

The overall release profile of such a formulation can be adjusted using, for example, multiple particle types or multiple layers. For example, in an aspect, a first bacterial mixture (or first dose of a bacterial mixture) can be formulated for release in, for example, the small intestine (e.g., one or more of duodenum, jejunum, ileum), whereas the second bacterial mixture (or second dose of the bacterial mixture) is formulated for delayed release in, for example, the large intestine (e.g., one or more of cecum, ascending, transverse, descending or sigmoid portions of the colon, and rectum). In another example, the first bacterial mixture (or first dose of a bacterial mixture) can be formulated for release in, for example, the small intestine (e.g., one or more of duodenum, jejunum, ileum), whereas the second bacterial mixture (or second dose of a bacterial mixture) is formulated for delayed release in, for example, another part of the small intestine (e.g., one or more of duodenum, jejunum, ileum). In another aspect, the first bacterial mixture (or first dose of a bacterial mixture) can be formulated for release in, for example, the large intestine (e.g., one or more of cecum, ascending, transverse, descending or sigmoid portions of the colon, and rectum), whereas the second bacterial mixture (or second dose of the bacterial mixture) is formulated for delayed release in, for example, another part of the large intestine (e.g., one or more of cecum, ascending, transverse, descending or sigmoid portions of the colon, and rectum). In various aspects, the composition and/or formulation can release at least one dose, at least two doses, at least three doses, at least four doses, or at least five doses of the bacterial mixture at different locations along the intestines, at different times, and/or at different pH. Likewise, in various aspects, the composition and/or formulation can release at least one bacterial mixture, at least two bacterial mixtures, at least three bacterial mixtures, at least four bacterial mixtures, or at least five bacterial mixtures at different locations along the intestines, at different times, and/or at different pH.

In another aspect, a delayed or gradual enteric release formulation comprises the use of a bilayer tablet or capsule which comprises a first layer comprising a polyalkylene oxide, a polyvinylpyrrolidone, a lubricant, or a mixture thereof, and a second osmotic push layer comprising polyethylene oxide, carboxy-methylcellulose, or both. In an aspect, a delayed or gradual enteric release formulation comprises the use of a release-retarding matrix material selected from the group consisting of an acrylic polymer, a cellulose, a wax, a fatty acid, shellac, zein, hydrogenated vegetable oil, hydrogenated castor oil, polyvinylpyrrolidine, a vinyl acetate copolymer, a vinyl alcohol copolymer, polyethylene oxide, an acrylic acid and methacrylic acid copolymer, a methyl methacrylate copolymer, an ethoxyethyl methacrylate polymer, a cyanoethyl methacrylate polymer, an aminoalkyl methacrylate copolymer, a poly(acrylic acid), a poly(methacrylic acid), a methacrylic acid alkylamide copolymer, a poly(methyl methacrylate), a poly(methacrylic acid anhydride), a methyl methacrylate polymer, a polymethacrylate, a poly(methyl methacrylate) copolymer, a polyacrylamide, an aminoalkyl methacrylate copolymer, a glycidyl methacrylate copolymer, a methyl cellulose, an ethylcellulose, a carboxymethylcellulose, a hydroxypropylmethylcellulose, a hydroxymethyl cellulose, a hydroxyethyl cellulose, a hydroxypropyl cellulose, a crosslinked sodium carboxymethylcellulose, a crosslinked hydroxypropylcellulose, a natural wax, a synthetic wax, a fatty alcohol, a fatty acid, a fatty acid ester, a fatty acid glyceride, a hydrogenated fat, a hydrocarbon wax, stearic acid, stearyl alcohol, beeswax, glycowax, castor wax, carnauba wax, a polylactic acid, polyglycolic acid, a co-polymer of lactic and glycolic acid, carboxymethyl starch, potassium methacrylate/divinylbenzene copolymer, crosslinked polyvinylpyrrolidone, poly inylalcohols, polyvinylalcohol copolymers, polyethylene glycols, non-crosslinked polyvinylpyrrolidone, polyvinylacetates, polyvinylacetate copolymers, or any combination thereof. In an aspect, a delayed or gradual enteric release formulation comprises the use of a microenvironment pH modifier.

It will be understood that a pharmaceutical composition described herein can comprise multiple distinct bacterial mixtures, for example to achieve different delivery location profiles for each bacterial mixture. In an aspect, a pharmaceutical composition comprises at least two bacterial mixtures, such that a first bacterial mixture comprises one or more bacterial isolates and a second bacterial mixture comprises a consortium of fecal bacteria. In an aspect, the second bacterial mixture further comprises one or more bacterial isolates that are different than the bacterial isolate(s) in the first bacterial mixture. Alternatively, the second bacterial mixture can consist essentially of the consortium of fecal bacteria. In another aspect, the first bacterial mixture can comprise only one bacterial isolate. A pharmaceutical composition can comprise any number of bacterial mixtures, for example one, two, three, four, five, six, seven, eight, nine, ten, or more than ten bacterial mixtures that each contain a different bacterial isolate, a different combination of bacterial isolates, a consortium of fecal bacteria, or a different combination of a consortium of fecal bacteria with one or more bacterial isolates.

In an aspect, a pharmaceutical composition can be a drench. In one aspect, a drench is prepared by choosing a saline-suspended form of a pharmaceutical composition. A water-soluble form of one ingredient can be used in conjunction with a water-insoluble form of the other by preparing a suspension of one with an aqueous solution of the other. Water-insoluble forms of either active ingredient may be prepared as a suspension or in some physiologically acceptable solvent such as polyethylene glycol. Suspensions of water-insoluble forms of either active ingredient can be prepared in oils such as peanut, corn, sesame oil or the like; in a glycol such as propylene glycol or a polyethylene glycol; or in water depending on the solubility of a particular active ingredient. Suitable physiologically acceptable adjuvants may be necessary in order to keep the active ingredients suspended. Adjuvants can include and be chosen from among the thickeners, such as carboxymethylcellulose, polyvinyl pyrrolidone, gelatin and the alginates. Surfactants generally will serve to suspend the active ingredients, particularly the fat-soluble propionate-enhancing compounds. Most useful for making suspensions in liquid nonsolvents are alkylphenol polyethylene oxide adducts, naphthalenesulfonates, alkylbenzene-sulfonates, and the polyoxyethylene sorbitan esters. In addition many substances, which affect the hydrophilicity, density and surface tension of the liquid, can assist in making suspensions in individual cases. For example, silicone anti-foams, glycols, sorbitol, and sugars can be useful suspending agents.

In some aspects, one or more bacterial isolates described herein are in the form of live, vegetative cells. In some aspects, one or more bacterial isolates described herein are in the form of spores. In some aspects, one or more bacterial isolates described herein are lyophilized. By way of non-limiting example, lyophilization can be via methods known in the art, including those described in U.S. Pat. No. 7,799,328, the contents of which are hereby incorporated by reference in their entirety. In some aspects, lyophilized bacterial mixtures described herein are placed in an enterically coated soft gel or capsule.

In various aspects, formulations can take the form of those described in one or more of U.S. Pat. Nos. 8,535,713 and 8,911,777 and US Patent Publication Nos. 20120141585, 20120141531, 2006/001896, 2007/0292523, 2008/0020018, 2008/0113031, 2010/0203120, 2010/0255087, 2010/0297221, 2011/0052645, 2013/0243873, 2013/0330411, 2014/0017313, and 2014/0234418, the contents of which are hereby incorporated by reference in their entirety.

In various aspects, formulations can take the form of those as described in International Patent Publication No. WO 2008/135090, the contents of which are hereby incorporated by reference in their entirety.

In various aspects, formulations can take the form of those described in one or more of U.S. Pat. Nos. 4,196,564; 4,196,565; 4,247,006; 4,250,997; 4,268,265; 5,317,849; 6,572,892; 7,712,634; 8,074,835; 8,398,912; 8,440,224; 8,557,294; 8,646,591; 8,739,812; 8,810,259; 8,852,631; and 8,911,788 and US Patent Publication Nos. 2014/0302132; 2014/0227357; 20140088202; 20130287842; 2013/0295188; 2013/0307962; and 20130184290, the contents of which are hereby incorporated by reference in their entirety.

### Administration and Dosage

It will be appreciated that the dose of a pharmaceutical composition or the bacterial cells therein (e.g., a bacterial mixture comprising one or more bacterial isolates and/or a consortium of fecal bacteria) will vary according to, for example, the particular dosage form, the mode of administration to a subject, the identity of a bacterial isolate, if any, in the composition, the number of bacterial isolates, if any, in the composition. These factors, as well as variables that may modify the activity of the bacteria in a bacterial mixture (e.g., subject body weight, sex and diet, time of administration, route of administration, rate of excretion, condition of the subject, drug combinations, genetic disposition and reaction sensitivities) can be taken into account by those skilled in the art to generate an effective dose or dosage regime for treatment or prevention of at least one symptom of a disorder described herein (e.g., a disorder associated with a gut dysbiosis that can be treated or prevented by administering one or more bile acid transforming bacteria). Administration can be carried out continuously or in one or more discrete doses within the maximum tolerated dose. Optimal administration rates for a given set of conditions can be ascertained by those skilled in the art using conventional dosage administration tests.

In various aspects, the dose of the pharmaceutical composition or the bacterial cells therein (e.g., a bacterial mixture comprising one or more bacterial isolates and/or a consortium of fecal bacteria) is effective to modulate a patient's microbiome to favor an ecological balance, so as to treat or prevent one or more symptoms of a disorder associated with a gut dysbiosis (e.g., by delivering one or more bile acid transforming bacteria to the intestine of the patient).

In one aspect, a pharmaceutically active or therapeutically effective dose of a bacterial isolate administered to a subject (i.e., in single or multiple administrations) to treat at least one symptom of a disorder (e.g., a disorder associated with a gut dysbiosis that can be treated or prevented by administration of at least one bile acid transforming bacteria) comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ CFUs of the bacterial isolate. In another aspect, a pharmaceutically active or therapeutically effective dose of a bacterial isolate administered to a subject (i.e., in single or multiple administrations) to treat at least one symptom of a disorder (e.g., a disorder associated with a gut dysbiosis that can be treated or prevented by administration of at least one bile acid transforming bacteria) comprises at most 10⁵, at most 10⁶, at most 10⁷, at most 10⁸, at most 10⁹, at most 10¹⁰, at most 10¹¹, at most 10¹², at most 10¹³, at most 10¹⁴, or at most 10¹⁵ CFUs of the bacterial isolate. In a further aspect, a pharmacologically active or therapeutically effective dose of a bacterial isolate administered to a subject (i.e., in single or multiple administrations) to treat at least one symptom of a disorder (e.g., a disorder associated with a gut dysbiosis that can be treated or prevented by administration of at least one bile acid transforming bacteria) is selected from the group consisting of: from 10⁸ CFUs to 10¹⁴ CFUs, from 10⁹ CFUs to 10¹³ CFUs, from 10¹⁰ CFUs to 10¹² CFUs, from 10¹⁰ CFUs to 10¹¹ CFUs, from 10⁹ CFUs to 10¹⁴ CFUs, from 10⁹ CFUs to 10¹² CFUs, from 10⁹ CFUs to 10¹¹ CFUs, from 10⁹ CFUs to 10¹⁰ CFUs, from 10¹⁰ CFUs to 10¹⁴ CFUs, from 10¹⁰ CFUs to 10¹³ CFUs, from 10¹¹ CFUs to 10¹⁴ CFUs, from 10¹¹ CFUs to 10¹³ CFUs, from 10¹² CFUs to 10¹⁴ CFUs, and from 10¹³ CFUs to 10¹⁴ CFUs of the bacterial isolate.

In an aspect, a pharmaceutical composition comprises one or more bacterial isolates, with each bacterial isolate present in each unit dose at one of the foregoing pharmaceutically active or therapeutically effective doses in a unit weight of about 0.2, 0.4, 0.6, 0.8 or 1.0 gram, or a unit volume of about 0.2, 0.4, 0.6, 0.8 or 1.0 milliliter.

In one aspect, a pharmaceutically active or therapeutically effective dose of a bacterial isolate administered to a subject (i.e., in single or multiple administrations) to treat at least one symptom of a disorder comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ cells or spores of the bacterial isolate. In another aspect, a pharmaceutically active or therapeutically effective dose of a bacterial isolate administered to a subject i.e. in single or multiple administrations) to treat at least one symptom of a disorder comprises at most 10⁵, at most 10⁶, at most 10⁷, at most 10⁸, at most 10⁹, at most 10¹⁰, at most 10¹¹, at most 10¹², at most 10¹³, at most 10¹⁴, or at most 10¹⁵ total cells or spores of the bacterial isolate. In a further aspect, a pharmacologically active or therapeutically effective dose of a bacterial isolate administered to a subject (i.e., in single or multiple administrations) to treat at least one symptom of a disorder (e.g., a disorder associated with a gut dysbiosis that can be treated or prevented by administration of at least one bile acid transforming bacteria) is selected from the group consisting of: from 10⁸ to 10¹⁴, from 10⁹ to 10¹³, from 10¹⁰ to 10¹², from 10¹⁰ to 10¹¹, from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores of the bacterial isolate.

In an aspect, the pharmaceutically active or therapeutically effective dose cell count of a bacterial isolate is directed to live cells. In one aspect, a pharmaceutical composition comprises one or more bacterial isolates, with each bacterial isolates present in each dosage unit at one of the foregoing pharmaceutically active or therapeutically effective doses in a unit weight of about 0.2, 0.4, 0.6, 0.8 or 1.0 gram, or a unit volume of about 0.2, 0.4, 0.6, 0.8 or 1.0 milliliter.

In an aspect, a pharmaceutical composition described herein is in the form of a capsule, and each capsule comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ cells or spores of a bacterial isolate. In an aspect, a pharmaceutical composition described herein is in the form of a capsule, and each capsule comprises from 10⁸ to 10¹⁴, from 10⁹ to 10¹³, from 10¹⁰ to 10¹², from 10¹⁰ to 10¹¹, from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, or from 10¹³ to 10¹⁴ cells or spores of a bacterial isolate.

In one aspect, a pharmaceutically active or therapeutically effective dose of a consortium of fecal bacteria administered to a subject (i.e., in single or multiple administrations) to treat at least one symptom of a disorder (e.g., associated with a gut dysbiosis) comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ CFUs of the consortium of fecal bacteria. In another aspect, a pharmaceutically active or therapeutically effective dose of a consortium of fecal bacteria administered to a subject (i.e., in single or multiple administrations) to treat at least one symptom of a disorder (e.g., associated with a gut dysbiosis) comprises at most 10⁵, at most 10⁶, at most 10⁷, at most 10⁸, at most 10⁹, at most 10¹⁰, at most 10¹¹, at most 10¹², at most 10¹³, at most 10¹⁴, or at most 10¹⁵ CFUs of the consortium of fecal bacteria. In a further aspect, a pharmacologically active or therapeutically effective dose of a consortium of fecal bacteria administered to a subject (i.e., in single or multiple administrations) to treat at least one symptom of a disorder (e.g., associated with a gut dysbiosis) is selected from the group consisting of: from 10⁸ CFUs to 10¹⁴ CFUs, from 10⁹ CFUs to 10¹³ CFUs, from 10¹⁰ CFUs to 10¹² CFUs, from 10¹⁰ CFUs to 10¹¹ CFUs, from 10⁹ CFUs to 10¹⁴ CFUs, from 10⁹ CFUs to 10¹² CFUs, from 10⁹ CFUs to 10¹¹ CFUs, from 10⁹ CFUs to 10¹⁰ CFUs, from 10¹⁰ CFUs to 10¹⁴ CFUs, from 10¹⁰ CFUs to 10¹³ CFUs, from 10¹¹ CFUs to 10¹⁴ CFUs, from 10¹¹ CFUs to 10¹³ CFUs, from 10¹² CFUs to 10¹⁴ CFUs, and from 10¹³ CFUs to 10¹⁴ CFUs of the consortium of fecal bacteria.

In an aspect, a consortium of fecal bacteria are present in each unit dose of a pharmaceutical composition at one of the foregoing pharmaceutically active or therapeutically effective doses in a unit weight of about 0.2, 0.4, 0.6, 0.8 or 1.0 gram, or a unit volume of about 0.2, 0.4, 0.6, 0.8 or 1.0 milliliter.

In one aspect, a pharmaceutically active or therapeutically effective dose of a consortium of fecal bacteria administered to a subject (i.e., in single or multiple administrations) to treat at least one symptom of a disorder (e.g., associated with a gut dysbiosis) comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ cells or spores of the consortium of fecal bacteria. In another aspect, a pharmaceutically active or therapeutically effective dose of a consortium of fecal bacteria administered to a subject i.e. in single or multiple administrations) to treat at least one symptom of a disorder (e.g., associated with a gut dysbiosis) comprises at most 10⁵, at most 10⁶, at most 10⁷, at most 10⁸, at most 10⁹, at most 10¹⁰, at most 10¹¹, at most 10¹², at most 10¹³, at most 10¹⁴, or at most 10¹⁵ total cells or spores of the preparation of a consortium of fecal bacteria. In a further aspect, a pharmacologically active or therapeutically effective dose of a preparation of a consortium of fecal bacteria administered to a subject (i.e., in single or multiple administrations) to treat at least one symptom of a disorder (e.g., associated with a gut dysbiosis) is selected from the group consisting of: from 10⁸ to 10¹⁴, from 10⁹ to 10¹³, from 10¹⁰ to 10¹², from 10¹⁰ to 10¹¹, from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, and from 10¹³ to 10¹⁴ cells or spores of the consortium of fecal bacteria.

In an aspect, the pharmaceutically active or therapeutically effective dose cell count of a consortium of fecal bacteria is directed to live cells. In one aspect, a consortium of fecal bacteria is present in each unit dose of a pharmaceutical composition at one of the foregoing pharmaceutically active or therapeutically effective doses in a unit weight of about 0.2, 0.4, 0.6, 0.8 or 1.0 gram, or a unit volume of about 0.2, 0.4, 0.6, 0.8 or 1.0 milliliter.

In an aspect, a pharmaceutical composition described herein is in the form of a capsule, and each capsule comprises at least 10⁵, at least 10⁶, at least 10⁷, at least 10⁸, at least 10⁹, at least 10¹⁰, at least 10¹¹, at least 10¹², at least 10¹³, at least 10¹⁴, or at least 10¹⁵ cells or spores of a consortium of fecal bacteria. In an aspect, a pharmaceutical composition described herein is in the form of a capsule, and each capsule comprises from 10⁸ to 10⁴, from 10⁹ to 10¹³, from 10¹⁰ to 10¹², from 10¹⁰ to 10¹¹, from 10⁹ to 10¹⁴, from 10⁹ to 10¹², from 10⁹ to 10¹¹, from 10⁹ to 10¹⁰, from 10¹⁰ to 10¹⁴, from 10¹⁰ to 10¹³, from 10¹¹ to 10¹⁴, from 10¹¹ to 10¹³, from 10¹² to 10¹⁴, or from 10¹³ to 10¹⁴ cells or spores of a consortium of fecal bacteria.

A subject can be administered one or more bacterial isolates (e.g., comprising at least one bile acid transforming bacteria) combined with a consortium of fecal bacteria for treatment of one or more symptoms of a disorder (e.g., a disorder associated with a gut dysbiosis that can be treated or prevented by administration of at least one bile acid transforming bacteria). In such cases, the bacterial isolate(s) and consortium of fecal bacteria can be administered to the subject together in the same pharmaceutical composition, or in separate compositions. Further, a pharmaceutical composition (e.g., comprising one or more bacterial isolates, a consortium of fecal bacteria, or both) can be administered to the subject in a single unit dose or multiple unit doses, for example as part of a dosage regime. In an aspect, the dosage of the consortium of fecal bacteria (e.g. measured by CFU or cell/spore count) administered to a subject is greater than the dosage of the bacterial isolate. Alternatively, the consortium of fecal bacteria (e.g. measured by CFU or cell/spore count) administered to the subject can be less than the dosage of the bacterial isolate. In another aspect, the dosage of the consortium of fecal bacteria (e.g. measured by CFU or cell/spore count) can be about the same as the dosage of the bacterial isolate. For example, in an aspect a subject can be administered a bacterial isolate (e.g. comprising at least one bile acid transforming bacteria) at a dosage of about 10¹⁰ cells and a consortium of fecal bacteria at a dosage of about 10¹⁰ cells to treat or prevent one or more symptoms of a disorder described herein.

In an aspect, the number of cells of a bacterial isolate (e.g., comprising at least one bile acid transforming bacteria) administered to a subject to treat one or more symptoms of a disorder described herein (e.g., a disorder associated with a gut dysbiosis that can be treated or prevented by administration of at least one bile acid transforming bacteria) is about the same or greater than the total number of cells of a consortium of fecal bacteria administered to the subject. Alternatively, the number of cells of a bacterial isolate administered to a subject to treat one or more symptoms of a disorder (e.g., a disorder associated with a gut dysbiosis that can be treated or prevented by administration of at least one bile acid transforming bacteria) can be about the same or less than the total number of cells of a consortium of fecal bacteria administered to the subject.

In an aspect, a pharmaceutical composition comprises a bacterial mixture that comprises multiple bacterial isolates (e.g., at least one of which comprises one or more bile acid transforming bacteria). In another aspect, at least two bacterial isolates are present at about the same amount or dosage (e.g., about the same number of viable cells or spores, or about the same CFUs). In another aspect, at least three bacterial isolates, at least four bacterial isolates, at least five bacterial isolates, at least six bacterial isolates, at least seven bacterial isolates, at least eight bacterial isolates, at least nine bacterial isolates, at least ten bacterial isolates, or more than ten bacterial isolates are present in the pharmaceutical composition at about the same amount or dosage (e.g., about the same number of viable cells or spores, or about the same CFUs). In another aspect, all of the bacterial isolates in a bacterial mixture are present in about the same amounts.

In an aspect, a pharmaceutical composition comprises a bacterial mixture comprising multiple bacterial isolates, and at least two of the multiple bacterial isolates are present at different amounts or dosages (e.g., different numbers of viable cells or spores, or different CFUs). In another aspect, at least three, at least four, at least five, at least six, at least seven, at least eight, at least nine, at least ten, or more than ten bacterial isolates are present in the bacterial mixture at different amounts or dosages.

A pharmaceutical composition can comprise a bacterial mixture comprising multiple bacterial isolates (e.g., at least one of which comprising a bile acid transforming bacteria) in combination with a consortium of fecal bacteria. In an aspect, each bacterial isolate is present in the composition at an amount or dosage that is greater than the amount or dosage of the consortium of fecal bacteria (e.g., measured as numbers of viable cells or spores, or CFUs). In another aspect, each bacterial isolate is present in the composition at an amount or dosage that is less than the amount or dosage of the consortium of fecal bacteria (e.g., measured as numbers of viable cells or spores, or CFUs). In another aspect, at least one bacterial isolate is present in the composition at an amount or dosage that is greater than the amount or dosage of the consortium of fecal bacteria, and at least one bacterial isolate is present in the composition at an amount or dosage that is less than the amount or dosage of the consortium of fecal bacteria (e.g., measured as numbers of viable cells or spores, or CFUs).

In an aspect, a pharmaceutical composition comprises one or more bacterial isolates at an amount or dosage which is at or above the minimum amount or dosage of the bacterial isolate required to be administered to a subject for engraftment of the bacterial isolate to occur in the intestine of the subject. For example, a minimum dosage of the bacterial isolate required for engraftment of the bacterial isolate into the intestine of the subject can be at least 10⁶ cells, at least 10⁷ cells, at least 10⁸ cells, at least 10⁹ cells, at least 10¹⁰ cells, at least 10¹¹ cells, or at least 10¹² cells. In an aspect a first and second bacterial isolate of a microbial cocktail engraft in the intestine of a subject at different minimal dosages or amounts, and a dosage or amount of each of the first and second bacterial isolate in the microbial cocktail varies corresponding to the respective minimal dosage or amount required for engraftment of the respective bacterial isolate.

Individual doses of the pharmaceutical composition (e.g., comprising a bacterial mixture) can be administered in unit dosage forms (e.g., tablets or capsules) containing, for example, from about 0.01 mg to about 5,000 mg, from about 0.01 mg to about 4,000 mg, from about 0.01 mg to about 3,000 mg, from about 0.01 mg to about 2,000 mg, from about 0.01 mg to about 1,000 mg, from about 0.01 mg to about 950 mg, from about 0.01 mg to about 900 mg, from about 0.01 mg to about 850 mg, from about 0.01 mg to about 800 mg, from about 0.01 mg to about 750 mg, from about 0.01 mg to about 700 mg, from about 0.01 mg to about 650 mg, from about 0.01 mg to about 600 mg, from about 0.01 mg to about 550 mg, from about 0.01 mg to about 500 mg, from about 0.01 mg to about 450 mg, from about 0.01 mg to about 400 mg, from about 0.01 mg to about 350 mg, from about 0.01 mg to about 300 mg, from about 0.01 mg to about 250 mg, from about 0.01 mg to about 200 mg, from about 0.01 mg to about 150 mg, from about 0.01 mg to about 100 mg, from about 0.1 mg to about 90 mg, from about 0.1 mg to about 80 mg, from about 0.1 mg to about 70 mg, from about 0.1 mg to about 60 mg, from about 0.1 mg to about 50 mg, from about 0.1 mg to about 40 mg, from about 0.1 mg to about 30 mg, from about 0.1 mg to about 20 mg, from about 0.1 mg to about 10 mg, from about 0.1 mg to about 5 mg, from about 0.1 mg to about 3 mg, from about 0.1 mg to about 1 mg of the active ingredient per unit dosage form, or from about 5 mg to about 80 mg per unit dosage form. For example, a unit dosage form can include about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 2,000 mg, about 3,000 mg, about 4,000 mg, or about 5,000 mg of the active ingredient, inclusive of all values and ranges therebetween.

In an aspect, the pharmaceutical composition (e.g., comprising a bacterial mixture) is administered at an amount of from about 0.01 mg to about 100 mg daily, an amount of from about 0.01 mg to about 5,000 mg daily, about 0.01 mg to about 4,000 mg daily, about 0.01 mg to about 3,000 mg daily, about 0.01 mg to about 2,000 mg daily, about 0.01 mg to about 1,000 mg daily, from about 0.01 mg to about 950 mg daily, from about 0.01 mg to about 900 mg daily, from about 0.01 mg to about 850 mg daily, from about 0.01 mg to about 800 mg daily, from about 0.01 mg to about 750 mg daily, from about 0.01 mg to about 700 mg daily, from about 0.01 mg to about 650 mg daily, from about 0.01 mg to about 600 mg daily, from about 0.01 mg to about 550 mg daily, from about 0.01 mg to about 500 mg daily, from about 0.01 mg to about 450 mg daily, from about 0.01 mg to about 400 mg daily, from about 0.01 mg to about 350 mg daily, from about 0.01 mg to about 300 mg daily, from about 0.01 mg to about 250 mg daily, from about 0.01 mg to about 200 mg daily, from about 0.01 mg to about 150 mg daily, from about 0.1 mg to about 100 mg daily, from about 0.1 mg to about 95 mg daily, from about 0.1 mg to about 90 mg daily, from about 0.1 mg to about 85 mg daily, from about 0.1 mg to about 80 mg daily, from about 0.1 mg to about 75 mg daily, from about 0.1 mg to about 70 mg daily, from about 0.1 mg to about 65 mg daily, from about 0.1 mg to about 60 mg daily, from about 0.1 mg to about 55 mg daily, from about 0.1 mg to about 50 mg daily, from about 0.1 mg to about 45 mg daily, from about 0.1 mg to about 40 mg daily, from about 0.1 mg to about 35 mg daily, from about 0.1 mg to about 30 mg daily, from about 0.1 mg to about 25 mg daily, from about 0.1 mg to about 20 mg daily, from about 0.1 mg to about 15 mg daily, from about 0.1 mg to about 10 mg daily, from about 0.1 mg to about 5 mg daily, from about 0.1 mg to about 3 mg daily, from about 0.1 mg to about 1 mg daily, or from about 5 mg to about 80 mg daily. In various aspects, the bacterial mixture (and/or additional therapeutic agents) is administered at a daily dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1,000 mg, about 2,000 mg, about 3,000 mg, about 4,000 mg, or about 5,000 mg inclusive of all values and ranges therebetween.

In some aspects, a suitable dosage of the pharmaceutical composition (e.g., comprising a bacterial mixture) is in a range of about 0.01 mg/kg to about 100 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, about 20 mg/kg body weight, about 30 mg/kg body weight, about 40 mg/kg body weight, about 50 mg/kg body weight, about 60 mg/kg body weight, about 70 mg/kg body weight, about 80 mg/kg body weight, about 90 mg/kg body weight, or about 100 mg/kg body weight, inclusive of all values and ranges therebetween. In other aspects, a suitable dosage of the composition in a range of about 0.01 mg/kg to about 100 mg/kg of body weight, in a range of about 0.01 mg/kg to about 90 mg/kg of body weight, in a range of about 0.01 mg/kg to about 80 mg/kg of body weight, in a range of about 0.01 mg/kg to about 70 mg/kg of body weight, in a range of about 0.01 mg/kg to about 60 mg/kg of body weight, in a range of about 0.01 mg/kg to about 50 mg/kg of body weight, in a range of about 0.01 mg/kg to about 40 mg/kg of body weight, in a range of about 0.01 mg/kg to about 30 mg/kg of body weight, in a range of about 0.01 mg/kg to about 20 mg/kg of body weight, in a range of about 0.01 mg/kg to about 10 mg/kg of body weight, in a range of about 0.01 mg/kg to about 9 mg/kg of body weight, in a range of about 0.01 mg/kg to about 8 mg/kg of body weight, in a range of about 0.01 mg/kg to about 7 mg/kg of body weight, in a range of 0.01 mg/kg to about 6 mg/kg of body weight, in a range of about 0.05 mg/kg to about 5 mg/kg of body weight, in a range of about 0.05 mg/kg to about 4 mg/kg of body weight, in a range of about 0.05 mg/kg to about 3 mg/kg of body weight, in a range of about 0.05 mg/kg to about 2 mg/kg of body weight, in a range of about 0.05 mg/kg to about 1.5 mg/kg of body weight, or in a range of about 0.05 mg/kg to about 1 mg/kg of body weight.

In accordance with certain aspects, the pharmaceutical composition (e.g., comprising a bacterial mixture) can be administered, for example, more than once daily, about once per day, about every other day, about every third day, about once a week, about once every two weeks, about once every month, about once every two months, about once every three months, about once every six months, or about once every year.

In an aspect, a pharmaceutical composition can be administered to a patient in need thereof at least once daily for at least two consecutive days. In another aspect, a pharmaceutical composition is administered at least once daily for at least 3, 4, 5, 6, 7, 8, 9, 10, 11, 12, 13, 14, or 15 consecutive days. In another aspect, a pharmaceutical composition is administered at least once daily for at least 1, 2, 3, 4, 5, 6, 7, 8, 9, 10, 11, or 12 consecutive weeks. In another aspect, a pharmaceutical composition is administered at least twice, three times, four times, or five times per week for at least 1, 2, 3, 4, 5, 6, 7, 8, 9, 10, 11, or 12 consecutive weeks. In another aspect, a pharmaceutical composition is administered at least once daily for at most 4, 5, 6, 7, 8, 9, 10, 11, 12, 13, 14, 15, 16, 17, 18, 19, or 20 consecutive days or weeks. In a further aspect, a pharmaceutical composition is administered at least once daily for at most 1, 2, 3, 4, 5, 6, 7, 8, 9, 10, 11, or 12 consecutive weeks or months. In yet another aspect, a pharmaceutical composition is administered at least once for at least 1, 2, 3, 4, 5, 6, 7, 8, 9, 10, 11, or 12 consecutive months or years, chronically for a subject's entire life span, or an indefinite period of time.

In an aspect, a pharmaceutical composition can be administered to a patient in need thereof at least twice daily for at least two consecutive days. In an aspect, a pharmaceutical composition is administered at least twice daily for at least 3, 4, 5, 6, 7, 8, 9, 10, 11, 12, 13, 14, or 15 consecutive days. In another aspect, a pharmaceutical composition is administered at least twice daily for at least 1, 2, 3, 4, 5, 6, 7, 8, 9, 10, 11, or 12 consecutive weeks. In another aspect, a pharmaceutical composition is administered at least twice daily for at most 4, 5, 6, 7, 8, 9, 10, 11, 12, 13, 14, 15, 16, 17, 18, 19, or 20 consecutive days or week. In another aspect, a pharmaceutical composition is administered at least twice daily for at most 1, 2, 3, 4, 5, 6, 7, 8, 9, 10, 11, or 12 consecutive weeks or months. In another aspect, a pharmaceutical composition is administered at least twice for at least 1, 2, 3, 4, 5, 6, 7, 8, 9, 10, 11, or 12 consecutive months or years, chronically for a subject's entire life span, or an indefinite period of time.

In an aspect of the present disclosure, a pharmaceutical composition can be administered to a patient in need thereof at least three times daily for at least two consecutive days. In an aspect, a pharmaceutical composition is administered at least three times daily for at least 3, 4, 5, 6, 7, 8, 9, 10, 11, 12, 13, 14, or 15 consecutive days. In an aspect, a pharmaceutical composition is administered at least three times daily for at least 1, 2, 3, 4, 5, 6, 7, 8, 9, 10, 11, or 12 consecutive weeks. In an aspect, a pharmaceutical composition is administered at least three times daily for at most 4, 5, 6, 7, 8, 9, 10, 11, 12, 13, 14, 15, 16, 17, 18, 19, or 20 consecutive days or weeks. In an aspect, a pharmaceutical composition is administered at least three times daily for at most 1, 2, 3, 4, 5, 6, 7, 8, 9, 10, 11, or 12 consecutive weeks or months. In an aspect, a pharmaceutical composition is administered at least three times for at least 1, 2, 3, 4, 5, 6, 7, 8, 9, 10, 11, or 12 consecutive months or years, chronically for a subject's entire life span, or an indefinite period of time.

In an aspect, a pharmaceutical composition can be administered to a patient in need thereof at a dosing schedule of at least once or twice daily for at least three consecutive days or weeks. In an aspect, a dose is administered at least once, twice, or three times daily for a period between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

In an aspect, a pharmaceutical composition can be administered to a patient in need thereof at a dosing schedule of once-a-week, twice-a-week, or thrice-a-week. The term “once-a-week” means that a dose is administered typically only once in a week, for example, on the same day of each week. “Twice-a-week” means that a dose is administered typically only two times in a week, for example, on the same two days of each weekly period. “Thrice-a-week” means that a dose is administered typically only three times in a week, for example, on the same three days of each weekly period.

In an aspect, a pharmaceutical composition can be administered to a patient in need thereof, wherein the administration comprises a first dosing schedule followed by a second dosing schedule. In an aspect, a first dosing schedule comprises a treatment or induction dose. In an aspect, a second dosing schedule comprises a maintenance dose. For example, a pharmaceutically active maintenance dose of a second dosage schedule can be lower than or equal to a pharmaceutically active induction dose of a first dosing schedule. In other examples, a maintenance dose of a second dosing schedule can be higher than an induction dose of a first dosing schedule.

At least one of a first and second dosing schedule for administering a pharmaceutical composition can comprise administration of the composition at least once daily for at least one day. In an aspect, at least one of a first or second dosing schedule comprises administration of the composition at least once daily for at least 2, 3, 4, 5, 6, 7, 8, 9, 10, 11, 12, 13, 14, or 15 consecutive days. In an aspect, at least one of a first or second dosing schedule comprises administration of the composition at least once daily for at least 1, 2, 3, 4, 5, 6, 7, 8, 9, 10, 11, or 12 consecutive weeks. In an aspect, at least one of a first or second dosing schedule comprises administration of the composition for at most 4, 5, 6, 7, 8, 9, 10, 11, 12, 13, 14, 15, 16, 17, 18, 19, or 20 consecutive days or weeks. In an aspect, at least one of a first or second dosing schedule comprises administration of the composition for at most 1, 2, 3, 4, 5, 6, 7, 8, 9, 10, 11, or 12 consecutive weeks or months. In an aspect, at least one of a first or second dosing schedule comprises administration of the composition for at least 1, 2, 3, 4, 5, 6, 7, 8, 9, 10, 11, or 12 consecutive months or years, chronically for a subject's entire life span, or an indefinite period of time.

In an aspect, at least one of a first or second dosing schedule used in a method can be once-a-week, twice-a-week, or thrice-a-week.

In an aspect, at least one of a first and second dosing schedule can last for at least about 2, 4, 6, 8, 10, 12, 18, 24, 36, 48, 72, or 96 months. In an aspect, a second dosing schedule lasts permanently, for a treated subject's entire life span, or an indefinite period of time. In an aspect, at least one of a first and second dosing schedule is a continuous dosing schedule. In an aspect, at least one of a first and second dosing schedule is an intermittent dosing schedule. In an aspect, at least one of a first and second dosing schedule is an intermittent dosing schedule comprising a treatment period of at least 1, 2, 3, 4, 5, 6, 7, 8, 9, 10, 11, 12, 13, or 14 days followed by a resting period of at least 1, 2, 3, 4, 5, 6, 7, 8, 9, 10, 11, 12, 13, or 14 days. In an aspect, at least one of a first and second dosing schedule comprises administering a dose every other day, every two days, or every 3, 4, 5, 6, 7, 8 days. In an aspect, a dose is administered for an extended period of time with or without titration (or otherwise changing the dosage or dosing schedule).

In an aspect, the interval between a first and a second dosing schedule is at least about 1, 2, 3, 4, 5, 6, or 7 days, or at least about 1, 2, 3, 4, 5, 6, 7, 8, 9, 10, 11, or 12 weeks, or at least about 1, 2, 3, 4, 6, 7, 8, 9, 10, 11, or 12 months.

In an aspect, a second dosing schedule (e.g., a maintenance dose) comprises a dosage about 2, 3, 4, 5, 6, 7, 8, 9, 10, 20, 30, 40, 50, 75, 100, 200, 400, 800, 1000, 5000 or more fold lower than the dosage used in a first dosing schedule (e.g., an initial induction dose). In another aspect, a second dosing schedule (e.g., a maintenance dosing schedule) has an equal or lower dosing frequency than a first dosing schedule (e.g., an initial treatment dosing schedule). In an aspect, a second dosing schedule (e.g., a maintenance dosing schedule) has a higher dosing interval than a first dosing schedule (e.g., an initial treatment dosing schedule).

In various aspects, methods described herein are useful in treatment of a human subject. In some aspects, the human is a pediatric human. In other aspects, the human is an adult human. In other aspects, the human is a geriatric human. In other aspects, the human may be referred to as a patient or subject. In some aspects, the human is a female. In some aspects, the human is a male.

In certain aspects, the human has an age in a range of from about 1 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In one aspect, a subject being treated is a human patient. In one aspect, a patient is a male patient. In one aspect, a patient is a female patient. In one aspect, a patient is a premature newborn. In one aspect, a patient is a term newborn. In one aspect, a patient is a neonate. In one aspect, a patient is an infant. In one aspect, a patient is a toddler. In one aspect, a patient is a young child. In one aspect, a patient is a child. In one aspect, a patient is an adolescent. In one aspect, a patient is a pediatric patient. In one aspect, a patient is a geriatric patient. In one aspect, a human patient is a child patient below about 18, 15, 12, 10, 8, 6, 4, 3, 2, or 1-year-old. In another aspect, a human patient is an adult patient. In another aspect, a human patient is an elderly patient. In a further aspect, a human patient is a patient above about 30, 35, 40, 45, 50, 55, 60, 65, 70, 75, 80, 85, 90, or 95 years old. In another aspect, a patient is about between 1 and 5, between 2 and 10, between 3 and 18, between 21 and 50, between 21 and 40, between 21 and 30, between 50 and 90, between 60 and 90, between 70 and 90, between 60 and 80, or between 65 and 75 years old. In one aspect, a patient is a young old patient (65-74 years). In one aspect, a patient is a middle old patient (75-84 years). In one aspect, a patient is an old patient (>85 years).

### Additional Therapeutic Agents and Co-Formulation

The pharmaceutical compositions described herein can include one or more therapeutic agents in addition to a bacterial mixture, which can be administered to a subject in need thereof in a method described herein. The additional therapeutic agent can be administered simultaneous or sequential with a bacterial mixture (e.g., comprising one or more bacterial isolates and/or a consortium of fecal bacteria) described herein. Further, the present compositions and formulations can comprise the additional therapeutic agent (e.g. via co-formulation). For example, the additional therapeutic agent, one or more bacterial isolates, and consortium of fecal bacteria can be combined into a single formulation.

In an aspect, the additional therapeutic agent and bacterial mixture are administered to a subject simultaneously. The term “simultaneously” as used herein, means that the additional therapeutic agent and the bacterial mixture are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and the bacterial mixture can be by simultaneous administration of a single formulation (e.g., a formulation comprising the additional therapeutic agent and a bacterial mixture) or of separate formulations (e.g., a first formulation including the additional therapeutic agent and a second formulation including the bacterial mixture).

Co-administration does not require an additional therapeutic agent to be administered simultaneously, if the timing of its administration is such that the pharmacological activities of the additional therapeutic agent and the bacterial mixture (e.g., comprising one or more bacterial isolates and/or a consortium of fecal bacteria) overlap in time. For example, the additional therapeutic agent and the bacterial mixture can be administered sequentially. The term “sequentially” as used herein means that the additional therapeutic agent and the bacterial mixture are administered with a time separation of more than about 60 minutes. For example, the time between the sequential administration of the additional therapeutic agent and the bacterial mixture can be more than about 60 minutes, more than about 2 hours, more than about 5 hours, more than about 10 hours, more than about 1 day, more than about 2 days, more than about 3 days, or more than about 1 week apart. The optimal administration times will depend on the rates of metabolism, excretion, and/or the pharmacodynamic activity of the additional therapeutic agent and the bacterial mixture being administered. Either of the additional therapeutic agent or the bacterial mixture can be administered first.

In a further aspect, the additional therapeutic agent and the bacterial mixture can be administered to a subject simultaneously but the release of additional therapeutic agent and the bacterial mixture from their respective dosage forms (or single unit dosage form if co-formulated) in the GI tract can occur sequentially.

Co-administration also does not require multiple additional therapeutic agents to be administered to the subject by the same route of administration as a bacterial mixture. Rather, each additional therapeutic agent can be administered by any appropriate route, for example, parenterally or non-parenterally.

In some aspects, the additional therapeutic agent is an agent used to treat or prevent one or more symptoms of a disorder described herein (e.g., a disorder associated with a gut dysbiosis that can be treated or prevented by administration of at least one bile acid transforming bacteria). In some aspects, the additional therapeutic agent is selected from the group consisting of isperidone, fluoxetine, aripiprazole, vitamin D, levocarnitine, and a combination thereof.

In some aspects, the additional therapeutic agent is an anti-inflammatory agent such as steroidal anti-inflammatory agents or non-steroidal anti-inflammatory agents (NSAIDS). Steroids, particularly the adrenal corticosteroids and their synthetic analogues, are well known in the art. Non-limiting examples of corticosteroids that can be administered to a subject as an additional therapeutic agent include hydroxyltriamcinolone, alpha-methyl dexamethasone, beta-methyl betamethasone, beclomethasone dipropionate, betamethasone benzoate, betamethasone dipropionate, betamethasone valerate, clobetasol valerate, desonide, desoxymethasone, dexamethasone, diflorasone diacetate, diflucortolone valerate, fluadrenolone, fluclorolone acetonide, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, clocortelone, clescinolone, dichlorisone, difluprednate, flucloronide, flunisolide, fluoromethalone, fluperolone, fluprednisolone, hydrocortisone, meprednisone, paramethasone, prednisolone, prednisone, beclomethasone dipropionate. (NSAIDS) that can be used, include but are not limited to, salicylic acid, acetyl salicylic acid, methyl salicylate, glycol salicylate, salicylmides, benzyl-2,5-diacetoxybenzoic acid, ibuprofen, fulindac, naproxen, ketoprofen, etofenamate, phenylbutazone, indomethacin, and a combination thereof. Additional anti-inflammatory agents are described, for example, in U.S. Pat. No. 4,537,776, the entire contents of which is incorporated by reference herein.

In some aspects, an additional therapeutic agent that can be incorporated into a pharmaceutical composition is a prebiotic. A prebiotic is a compound or compounds (e.g. comprising one or more nutrients) administered to a subject to promote the growth, proliferation, or activity of one or more microorganisms (e.g., bacteria) in the intestine of the subject (e.g., by providing a substrate to be metabolized by the one or more microorganisms). Without wishing to be bound by theory, prebiotics can be added to a pharmaceutical composition to nutritionally supplement bacteria in the endogenous microbiome of the subject and/or in the pharmaceutical composition itself, e.g., to stimulate the growth or activity of one or more species of a consortium of fecal bacteria and/or one or more bacterial isolates. Additionally, one or more prebiotics can be added to a composition to buffer against “shock” to bacteria cells when transitioning those cells to a new environment, for example, subsequent to the isolation, purification and/or culturing or fermentation of consortium of fecal bacteria, or before or after freezing, freeze-drying, spray-drying, reconstitution in solution and the like.

Non-limiting examples of prebiotics that can be added to a pharmaceutical composition include an amino acid (e.g., valine, leucine, isoleucine), lactic acid, ammonium nitrate, amylose, barley mulch, biotin, carbonate, cellulose, chitin, choline, fructooligosaccharides (FOSs), fructose, glucose, glycerol, heteropolysaccharide, histidine, homopolysaccharide, hydroxyapatite, inulin, isomaltulose, lactose, lactulose, maltodextrins, maltose, nitrogen, oligodextrose, oligofructose, oligofructose-enriched inulin, an oligosaccharide (e.g. comprising a galactooligosaccharide (GOS), trans-galactooligosaccharide, fructooligosaccharide (FOS), xylooligosaccharides (XOS), mannooligosaccharide, or chitooligosaccharide), pectin, phosphate salts, phosphorus, polydextroses, polyols, potash, potassium, sodium nitrate, starch, sucrose, sulfur, sun fiber, tagatose, thiamine, trehalose, vitamins, a water-soluble carbohydrate, a fermentable polysaccharide, a dietary fiber, resistant starch, barley, white navy bean powder, and a combination thereof. Illustrative prebiotics include complex carbohydrates, amino acids, peptides, or other essential nutritional components for the survival of the bacterial composition.

In an aspect, a subject is not pretreated with a prebiotic prior to treatment with a pharmaceutical composition. In another aspect, the pharmaceutical composition is not supplemented with a prebiotic.

In an aspect, a prebiotic can be included (e.g., in dry or liquid forms) in a pharmaceutical composition described herein, for example, comprising a bacterial mixture.

Alternately, or additionally, a prebiotic to be administered to a subject can be included (e.g., in dry or liquid forms) in a distinct pharmaceutical composition lacking a bacterial mixture.

A prebiotic can be administered to a subject before, contemporaneously with, and/or after administration of a pharmaceutical composition comprising a bacterial mixture, either in the same pharmaceutical composition or in a separate pharmaceutical composition.

A prebiotic can be provided and administered in a single dose or in multiple doses. When provided as a single dose, a single composition can comprise only one prebiotic or a mixture of prebiotics. When provided in multiple doses, each composition dosed to the subject can comprise a single prebiotic or a mixture of prebiotics, and/or a first composition dosed to the subject can comprise a different prebiotic or prebiotics than a second composition dosed to the subject.

As examples, when multiple doses are provided, a first composition comprising a prebiotic can include a first prebiotic, e.g., inulin, and a second composition can include a different prebiotic, e.g., fructooligosaccharide, with or without the first prebiotic. Alternately, a first composition can include a combination of prebiotics, e.g., inulin and fructooligosaccharide and a second composition can include a different combination of prebiotics, e.g., inulin and white navy bean powder. A first composition can include a combination of prebiotics and a second composition can include only one prebiotic.

The amount of prebiotic included in a composition depends on the specific prebiotic, the specific bacterial species targeted by the prebiotic, and/or the disease state of the subject/patient.

In some aspects, an additional therapeutic agent be incorporated into a pharmaceutical composition is an antidiarrheal agent. Non-limiting examples of antidiarrheal agents suitable for inclusion in a pharmaceutical composition described herein include, but are not limited to, DPP-IV inhibitors, natural opioids, such as tincture of opium, paregoric, and codeine, synthetic opioids, such as diphenoxylate, difenoxin and loperamide, bismuth subsalicylate, lanreotide, vapreotide and octreotide, motion antagonists, COX2 inhibitors like celecoxib, glutamine, thalidomide and traditional antidiarrheal remedies, such as kaolin, pectin, berberine and muscarinic agents, and a combination thereof.

In some aspects, the additional therapeutic agent incorporated into a pharmaceutical composition can be an analgesic. Analgesics useful in the compositions and methods described herein include, without limitation, morphine, codeine, heroine, methadone and related compounds, thebaine, orpiavine, and their derivatives, buprenorphine, the piperidines, morphinans, benzomorphans, tetrahydroisoquinolines, thiambutanes, benzylamines, tilidine, viminol, nefopam, capsaicin(8-methyl-N-vanillyl-6E-nonenamide), “synthetic” capsaicin(N-vanillylnonamide) and related compounds, and a combination thereof.

In some aspects, the additional therapeutic agent is an anti-bacterial agent, which includes, but is not limited to, cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); monobactam antibiotics (aztreonam); carbapenem antibiotics (ertapenem, doripenem, imipenem/cilastatin, and meropenem); and a combination thereof. In some aspects, the anti-bacterial agent can be any of the penicillin, cephalosporin, monobactam, and carbapenem antibiotics, or a combination thereof.

In one aspect, a method further comprises pretreating a subject with an antibiotic composition prior to administering a therapeutic bacterial mixture. In one aspect, an antibiotic composition administered herein comprises an antibiotic selected from the group consisting of rifabutin, clarithromycin, clofazimine, vancomycin, rifampicin, nitroimidazole, chloramphenicol, and a combination thereof. In another aspect, an antibiotic composition administered herein comprises an antibiotic selected from the group consisting of rifaximin, rifamycin derivative, rifampicin, rifabutin, rifapentine, rifalazil, bicozamycin, aminoglycoside, gentamycin, neomycin, streptomycin, paromomycin, verdamicin, mutamicin, sisomicin, netilmicin, retymicin, kanamycin, aztreonam, aztreonam macrolide, clarithromycin, dirithromycin, roxithromycin, telithromycin, azithromycin, bismuth subsalicylate, vancomycin, streptomycin, fidaxomicin, amikacin, arbekacin, neomycin, netilmicin, paromomycin, rhodostreptomycin, tobramycin, apramycin, and a combination thereof. In another aspect, a subject is not pretreated with an antibiotic composition prior to administering a bacterial mixture. In another aspect, the pharmaceutical composition is not supplemented with an antibiotic composition. In a further aspect, a method further comprises pretreating a subject with an anti-inflammatory drug prior to administration of a bacterial mixture. In yet another aspect, a subject is not pretreated with an anti-inflammatory drug prior to administering a bacterial or mixture. In another aspect, a bacterial mixture is not supplemented with an anti-inflammatory.

Delivery of an additional therapeutic agent can be targeted to various parts of the GI tract, as described herein.

### Use of Pharmaceutical Compositions

Disclosed herein are pharmaceutical compositions that can be administered to a subject to treat or prevent a condition, disorder or disease by increasing an amount of one or more bile acid transforming bacteria in the intestine of the subject, and as a result rescuing a deficiency in bile acid metabolism in the subject. In an aspect, administration of a composition described herein increases a concentration or level of a secondary bile acid in the intestine of the subject by administering bacteria in a bacterial mixture that have been selected to produce and/or secrete one or more secondary bile acids in the intestine of the subject following administration of the pharmaceutical composition. In an aspect, administration of a pharmaceutical composition comprising a bacterial mixture described herein treats or prevents a disorder by delivering one or more bile acid metabolizing bacteria to the intestine of the subject, via the engraftment in the intestine of bacteria contained within the administered bacterial mixture, and in certain embodiments the subsequent secretion of a secondary bile acid or intermediate or derivative thereof by the engrafted bacteria. Thus, the pharmaceutical compositions described herein are advantageous over compositions that administer a finite amount of a secondary bile acid alone (i.e., in the absence of a bile acid transforming bacteria), as administration of the bacterial mixtures and subsequent engraftment of the bacteria contained therein results in a continuous, sustained, long-term supply of secondary bile acids to a subject without the need for regular (e.g., daily) replenishing administrations of the applicable compound.

In aspects, a pharmaceutical composition can treat or prevent a disorder that is caused by a reduction or deficiency in a level of one or more secondary bile acids in the intestine of the subject. In such cases, administration of a composition can increase the level of the one or more secondary bile acids in the intestine to directly treat the disorder, resulting in a reduction in a severity of one or more symptoms associated with the disorder. In aspects, a pharmaceutical composition can treat or prevent a disorder that is not directly caused by a deficiency in a level of one or more secondary bile acids, but has one or more symptoms that are responsive to, or can benefit from, an increase in secondary bile acids in the intestine of the subject (e.g., the severity of one or more symptoms of the disorder is reduced as a result of an increase in one or more bile acid transforming bacteria in the intestine, even though the cause of the disorder may remain).

In various aspects, provided herein is a method of supplementing or modulating a microbiome of a subject in need thereof to provide or restore an ecological balance, comprising administering to the subject a composition described herein. For instance, in various aspects, there is provided methods of diminishing or inhibiting one or more pathogenic bacteria in the intestine of a patient by administering a composition described herein. In various aspects, administration of one or more bacterial isolates or a consortium of fecal bacteria described herein augments growth of at least one type of bacteria not detectably present in a patient's gastrointestinal (GI) tract prior to administration and, in various aspects, which is non-pathogenic.

In various aspects, provided herein is a method of restoring or enhancing ecological control over gut pathogens or pathobionts in a subject in need thereof, comprising administering to the subject a composition described herein.

In aspects, the pharmaceutical composition is for administration to a subject having a disorder related to an intestinal dysbiosis. In aspects, the disorder is selected from the group consisting of inflammatory bowel disease (IBD), irritable bowel syndrome (IBS), C. difficile infection (CDI), C. difficile-associated disease (CDAD), an antibiotic-induced adverse effect, and a combination thereof.

In various aspects, a method comprises administering a composition described herein to treat a disease or condition associated with GI dysbiosis in a subject in need thereof. In some aspects, the subject has inflammatory bowel disease (IBD), for example, Crohn's disease, colitis (e.g., ulcerative colitis or microscopic colitis), or pouchitis. IBD is a group of inflammatory conditions of the large intestine and, in some cases, the small intestine. Examples of LBD that can be treated by the compositions, formulations and methods described herein include, but are not limited to, Crohn's disease, ulcerative colitis, microscopic colitis, pouchitis, collagenous colitis, lymphocytic colitis, ischemic colitis, diversion colitis, Behçet's syndrome, infective colitis, and indeterminate colitis. In an aspect, provided herein is a method of treating ulcerative colitis comprising administering a composition described herein to a subject in need thereof. In another aspect, provided herein is a method of treating Crohn's Disease comprising administering a composition described herein to a subject in need thereof. In a further aspect, provided herein is a method of treating pouchitis comprising administering a composition described herein to a subject in need thereof.

In various aspects, a method comprises administering a composition described herein to treat ulcerative colitis (UC) in a subject in need thereof. UC is one form of IBD. It is a chronic disease of the colon, in which the lining of the colon becomes inflamed and develops tiny open sores, or ulcers, that produce pus and mucous. In some aspects, methods described herein can ameliorate, reduce, or eliminate the inflammation and/or ulceration associated with UC. In some aspects, methods described herein can ameliorate, reduce, or eliminate one or more symptoms associated with UC including but not limited to, abdominal discomfort or pain, frequent emptying of the colon, lose and urgent bowel movements, persistent diarrhea, bloody stool, loss of appetite, and weight loss. In some aspects, methods described herein can reduce or prevent the delay in growth and development in children afflicted with UC.

In various aspects, a method comprises administering a composition described herein to treat UC in a subject in need thereof. For example, a successful treatment of the subject can be measured using the indices below, e.g., the present methods cause a subject's activity score threshold to change from severe to moderate, mild, or remission; or cause a patient's score to change from moderate to mild or remission; or cause a patient's score to change from mild to remission (see Table 2).

TABLE 2

| | Parameters | Scoring | Activity score thresholds | | | |
|---|---|---|---|---|---|---|
| Index | assessed | system | Remission | Mild | Moderate | Severe |
| Mayo score | Stool frequency | Cumulative | 0-2 | 3-5 | 6-10 | 11-12 |
| | Rectal bleeding | score | | | | |
| | Physician's global | | | | | |
| | assessment | | | | | |
| | Sigmoidoscopy | | | | | |
| UCDAI | Stool frequency | Cumulative | 0-2 | 3-8 | | 9-12 |
| | Rectal bleeding | score | | | | |
| | Physician's global | | | | | |
| | assessment | | | | | |
| | Sigmoidoscopy | | | | | |
| Rachmilewitz | Bowel movement | Cumulative | 0-4 | 5-10 | 11-17 | >17 |
| score (CAI) | frequency | score | | | | |
| | Blood in stools | | | | | |
| | Physician's global | | | | | |
| | assessment | | | | | |
| | Abdominal | | | | | |
| | pain/cramps | | | | | |
| | Temperature | | | | | |
| | EIMs | | | | | |
| | Laboratory findings | | | | | |
| | (ESR, hemoglobin) | | | | | |
| Powell-Tuck | Well-being | Cumulative | ≤3 | 4-10 | 11-14 | >14 |
| index (St | Abdominal pain | score | | | | |
| Mark's index) | Bowel movement | | | | | |
| | frequency | | | | | |
| | Stool consistency | | | | | |
| | Bleeding | | | | | |
| | Anorexia | | | | | |
| | Nausea/vomiting | | | | | |
| | Abdominal | | | | | |
| | tenderness | | | | | |
| | Eye, joint, mouth, | | | | | |
| | or skin | | | | | |
| | complications | | | | | |
| | Temperature | | | | | |
| | Sigmoidoscopy | | | | | |
| SCCAI | Bowel movement | Cumulative | ≤2 | 3-20 | | |
| (Walmsley) | frequency (day) | score | <2.5 | | | |
| | Bowel movement | | | | | |
| | frequency (night) | | | | | |
| | Urgency of | | | | | |
| | defecation | | | | | |
| | Blood in stool | | | | | |
| | Well-being | | | | | |
| | Extracolonic | | | | | |
| | features | | | | | |
| Lichtiger index | Diarrhea frequency | Cumulative | ≤3 | 4-8 | 9-14 | >14 |
| | Nocturnal diarrhea | score | | | | |
| | Visible blood (% of | | | | | |
| | movements) | | | | | |
| | Fecal incontinence | | | | | |
| | Abdominal | | | | | |
| | pain/cramping | | | | | |
| | Well-being | | | | | |
| | Abdominal | | | | | |
| | tenderness | | | | | |
| | Need for | | | | | |
| | antidiarrheal | | | | | |
| | medications | | | | | |
| Seo index | Bowel movement | Cumulative | <108 | <150 | 150-220 | >220 |
| | frequency | score with | <120 | | | |
| | Blood in stool | components | | | | |
| | ESR | given | | | | |
| | Hemoglobin | different | | | | |
| | Albumin | weightings | | | | |
| | | (+ constant to | | | | |
| | | yield a mean | | | | |
| | | value as close | | | | |
| | | as possible to | | | | |
| | | Truelove- | | | | |
| | | Witts criteria) | | | | |

In some aspects, a method comprises administering a pharmaceutical composition described herein to treat irritable bowel syndrome (IBS) in a subject in need thereof. IBS is a common disorder that affects the colon and can cause cramping, abdominal pain, bloating, gas, diarrhea and constipation. IBS is classified based on the predominant symptom of diarrhea (IBS with predominant diarrhea, IBS-D), constipation (IBS with predominant constipation, IBS-C) or mixed symptoms (IBS with alternating constipation and diarrhea, IBS-A). Methods described herein can be effective in treating one or more of IBS-D, IBS-C, and/or IBS-A. In some aspects, methods described herein (e.g., comprising administering a composition described herein) can reduce or eliminate one or more symptoms associated with one or more of IBS-D, IBS-C, and/or IBS-A.

In aspects, a method comprises administering a pharmaceutical composition described herein to treat or prevent a disease/disorder associated with an abnormal enteric microflora (e.g. intestinal dysbiosis) in a subject in need thereof. The disease/disorder can be selected from a gastro-intestinal disorder including irritable bowel syndrome or spastic colon, Functional Bowel Disease (FBD), including constipation predominant FBD, pain predominant FBD, upper abdominal FBD, Nonulcer Dyspepsia (NUD), gastro-esophageal reflux, inflammatory bowel disease including Crohn's disease, ulcerative colitis, indeterminate colitis, collagenous colitis, microscopic colitis, chronic Clostridium difficile infection, pseudomembranous colitis, mucous colitis, antibiotic associated colitis, idiopathic or simple constipation, diverticular disease, AIDS enteropathy, small bowel bacterial overgrowth, coeliac disease, polyposis coil, colonic polyps, chronic idiopathic pseudo obstructive syndrome, and toxic megacolon.

In aspects, a method comprises administering a composition described herein to treat or prevent a disorder associated with a liver disorder in a subject in need thereof. Non-limiting examples of a liver disorder include nonalcoholic steatohepatitis (NASH), hepatic encephalopathy, hepatitis B, primary biliary cirrhosis, Primary Sclerosing Cholangitis (PSC), fatty liver, and cryptogenic cirrhosis. In aspects, such diseases/disorders are related to an intestinal dysbiosis of a subject.

In aspects, a method comprises administering a composition described herein to treat or prevent a rheumatic disorder in a subject in need thereof. Non-limiting examples of a rheumatic disorder include rheumatoid arthritis, non-rheumatoid arthritis, non-rheumatoid factor positive arthritis, ankylosing spondylitis, Lyme disease, and Reiter's syndrome. In aspects, such diseases/disorders are related to an intestinal dysbiosis of a subject.

In aspects, a method comprises administering a composition described herein to treat or prevent an immune-mediated disorder in a subject in need thereof. Non-limiting examples of an immune-mediated disorder include glomerulonephritis, hemolytic uraemic syndrome, juvenile diabetes mellitus, mixed cryoglobulinaemia, polyarteritis, familial Mediterranean fever, amyloidosis, scleroderma, systemic lupus erythematosus, and Beheets syndrome. In aspects, such diseases/disorders are related to an intestinal dysbiosis of a subject.

In aspects, a method comprises administering a composition described herein to treat or prevent an autoimmune disorder in a subject in need thereof. Non-limiting examples of an autoimmune disorder include Acute Disseminated Encephalomyelitis (ADEM), acute necrotizing hemorrhagic leukoencephalitis, Addison's disease, agammaglobulinemia, alopecia areata, amyloidosis, ankylosing spondylitis, anti-GBM/anti-TBM nephritis, Antiphospholipid Syndrome (APS), autoimmune angioedema, autoimmune aplastic anemia, autoimmune dysautonomia, autoimmune hemolytic anemia, autoimmune hepatitis, autoimmune hypedipidemia, autoimmune immunodeficiency, Autoimmune Inner Ear Disease (AIED), autoimmune myocarditis, autoimmune oophoritis, autoimmune pancreatitis, autoimmune retinopathy, Autoimmune Thrombocytopenic Purpura (ATP), autoimmune thyroid disease, autoimmune urticarial, axonal & neuronal neuropathies, Balo disease, Behcet's disease, bullous pemphigoid, cardiomyopathy, Castleman disease, celiac disease, Chagas disease, Chronic Inflammatory Demyelinating Polyneuropathy (CIDP), Chronic Recurrent Multifocal Ostomyelitis (CRMO), Churg-Strauss syndrome, cicatricial pemphigoid/benign mucosal pemphigoid, Crohn's disease, Cogan's syndrome, cold agglutinin disease, congenital heart block, Coxsackie myocarditis, CREST disease, essential mixed cryoglobulinemia, demyelinating neuropathies, dermatitis herpetiformis, dermatomyositis, Devic's disease (neuromyelitis optica), discoid lupus, Dressler's syndrome, endometriosis, eosinophilic esophagitis, eosinophilic fasciitis, erythema nodosum, experimental allergic encephalomyelitis, Evans syndrome, fibrosing alveolitis, giant cell arteritis (temporal arteritis), giant cell myocarditis, glomerulonephritis, Goodpasture's syndrome, Granulomatosis With Polyangiitis (GPA), Graves' disease, Guillain-Barre syndrome, Hashimoto's encephalitis, Hashimoto's thyroiditis, hemolytic anemia, Henoch-Schonlein purpura, herpes gestationis, hypogammaglobulinemia, idiopathic thrombocytopenic purpura (ITP), IgA nephropathy, IgG4-related sclerosing disease, immunoregulatory lipoproteins, inclusion body myositis, interstitial cystitis, juvenile arthritis, juvenile idiopathic arthritis, juvenile myositis, Kawasaki syndrome, Lambert-Eaton syndrome, leukocytoclastic vasculitis, lichen planus, lichen sclerosus, ligneous conjunctivitis, linear IgA disease (LAD), lupus (systemic lupus erythematosus), chronic Lyme disease, Meniere's disease, microscopic polyangiitis, Mixed Connective Tissue Disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, Paroxysmal Nocturnal Hemoglobinuria (PNH), Parry Romberg syndrome, Parsonnage-Turner syndrome, pars planitis (peripheral uveitis), pemphigus, peripheral neuropathy, perivenous encephalomyelitis, pernicious anemia, POEMS syndrome, polyarteritis nodosa, type I, II, & III autoimmune polyglandular syndromes, polymyalgia rheumatic, polymyositis, postmyocardial infarction syndrome, postpericardiotomy syndrome, progesterone dermatitis, primary biliary cirrhosis, Primary Sclerosing Cholangitis (PSC), psoriasis, psoriatic arthritis, idiopathic pulmonary fibrosis, pyoderma gangrenosum, pure red cell aplasia, Raynaud's phenomenon, reactive arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjögren's syndrome, sperm & testicular autoimmunity, stiff person syndrome, Subacute Bacterial Endocarditis (SBE), Susac's syndrome, sympathetic ophthalmia, Takayasu's arteritis, temporal arteritis/giant cell arteritis, Thrombocytopenic Purpura (TTP), Tolosa-Hunt syndrome, transverse myelitis, type 1 diabetes, asthma, ulcerative colitis, Undifferentiated Connective Tissue Disease (UCTD), uveitis, vasculitis, vesiculobullous dermatosis, vitiligo, and Wegener's granulomatosis. In aspects, such disorders are related to an intestinal dysbiosis of a subject.

In aspects, a method comprises administering a composition described herein to treat or prevent a neurological syndrome in a subject in need thereof. Non-limiting examples of a neurological syndrome include as chronic fatigue syndrome, migraine, multiple sclerosis, amyotrophic lateral sclerosis, myasthenia gravis, Gillain-Barré syndrome, Parkinson's disease, Alzheimer's disease, Chronic Inflammatory Demyelinating Polyneuropathy, and other degenerative disorders. In aspects, such syndromes are related to an intestinal dysbiosis of a subject.

In aspects, a method comprises administering a composition described herein to treat or prevent a psychiatric or mental disorder in a subject in need thereof. Non-limiting examples of a psychiatric or mental disorder include chronic depression, schizophrenia, psychotic disorders, manic depressive illness; regressive disorders including, Asperger's syndrome, Rett syndrome, Attention Deficit Hyperactivity Disorder (ADHD), Attention Deficit Disorder (ADD), the regressive disorder, autism, Sudden Infant Death Syndrome (SIDS), and anorexia nervosa. In aspects, such diseases/disorders are related to an intestinal dysbiosis of a subject.

In aspects, a method comprises administering a composition described herein to treat or prevent a dermatological condition in a subject in need thereof. Non-limiting examples of a dermatological condition include chronic urticaria, acne, eczema, atopic dermatitis, contact dermatitis, dermatitis herpetiformis and vasculitis disorders. In aspects, such diseases/disorders are related to an intestinal dysbiosis of a subject.

In aspects, a method comprises administering a composition described herein to treat or prevent a cardiovascular and/or vascular disorder in a subject in need thereof. In aspects, such diseases/disorders are related to an intestinal dysbiosis of a subject.

In aspects, a method comprises administering a composition described herein to treat or prevent a bloodstream infection (BSI) in a subject in need thereof. Patients at risk for such BSIs include but are not limited to solid organ transplant patients; chronic kidney disease patients, e.g., on hemodialysis; and oncology patients. In aspects, such BSIs are related to an intestinal dysbiosis of a subject.

In aspects, a method comprises administering a composition described herein to treat or prevent a catheter or intravascular-line infection (e.g., central-line infection) in a subject in need thereof. In aspects, such infections are related to an intestinal dysbiosis of a subject.

In aspects, a method comprises administering a composition described herein to treat or prevent a skin or soft tissue infection in a subject in need thereof. In aspects, such infections are related to an intestinal dysbiosis of a subject.

In aspects, a method comprises administering a composition described herein to treat or prevent a surgical-site infection in a subject in need thereof. In aspects, such infections are related to an intestinal dysbiosis of a subject.

In aspects, a method comprises administering a composition described herein to treat or prevent a urinary tract infection (e.g., antibiotic-resistant urinary tract infections and catheter-associated urinary tract infections) in a subject in need thereof. In aspects, such infections are related to an intestinal dysbiosis of a subject.

In aspects, a method comprises administering a composition described herein to treat or prevent a wound infection in a subject in need thereof. In aspects, such infections are related to an intestinal dysbiosis of a subject.

In aspects, a method comprises administering a composition described herein to treat or prevent an infection in a subject in need thereof. Non-limiting examples of an infection include an antibiotic-resistant infection and an antibiotic-sensitive infection. In aspects, such infections are related to an intestinal dysbiosis of a subject.

In aspects, the pharmaceutical compositions and methods described herein can treat or prevent meningitis. In aspects, the meningitis is related to an intestinal dysbiosis of a subject.

In aspects, a method comprises administering a composition described herein to treat or prevent pneumonia, e.g., ventilator-associated pneumonia in a subject in need thereof. In aspects, the pneumonia is related to an intestinal dysbiosis of a subject.

In aspects, the compositions, formulations and methods described herein can be used in patient populations who are in an outpatient setting, hospitalized, and/or in long-term care facilities. Such patient populations are at risk for nosocomial infections. In aspects, such infections are related to an intestinal dysbiosis of a subject.

In aspects, a method comprises administering a composition described herein to treat or prevent Primary Sclerosing Cholangitis (PSC) in a subject in need thereof. For example, one or more bacterial isolates provided in a pharmaceutical composition administered to the subject can replace a dysbiotic gut microbiome with a healthy community, thereby, at least, reducing bile duct inflammation and/or improving liver function.

In aspects, a method comprises administering a composition described herein to treat or prevent a diarrheal disease in a subject in need thereof. Non-limiting examples of a diarrheal disease include acute bloody diarrhea (e.g., dysentery), acute watery diarrhea (e.g., cholera), checkpoint inhibitor associated colitis, diarrhea due to food poisoning, persistent diarrhea, and traveler's diarrhea. In aspects, the diarrhea is related to an intestinal dysbiosis of a subject.

In various aspects, administration of a pharmaceutical composition described herein can reduce, ameliorate, or eliminate one or more symptom(s) associated with a herein-described condition, disease, or disorder. Exemplary symptoms include, but are not limited to, diarrhea, bloody stool, mouth sores, perianal disease, abdominal pain, abdominal cramping, fever, fatigue, weight loss, iron deficiency, anemia, appetite loss, weight loss, anorexia, delayed growth, delayed pubertal development, and inflammation of the skin, eyes, joints, liver, and bile ducts. In aspects, the symptom is related to an intestinal dysbiosis of a subject.

In some aspects, a method comprises administering a composition described herein to treat or prevent an infection by pathogenic bacteria and/or inhibit the growth or decrease the number of pathogenic bacteria in the GI tract of a subject in need thereof. In an aspect, the pathogenic bacteria is enterobacteria such as Salmonella. In various aspects, a method comprises administering a composition described herein to mitigate or prevent the overgrowth of various coliforms in a patient's gut (including coliforms that are virulent and/or antibiotic resistant). Illustrative coliforms include Citrobacter, Enterobacter, Hafnia, Kelbsiella, and Escherichia. In some aspects, the methods and compositions described herein prevent or diminish secondary infections with resistant organisms.

In still other aspects, a method comprises administering a composition described herein to treat or prevent an infectious disease of the intestines in a subject in need thereof. Non-limiting examples of an infectious disease of the intestine include CDI and/or a CDAD, nosocomial infection, secondary emergent infection, amebiasis, intestinal tuberculosis, or parasitic disorder. In some aspects, provided herein are methods for treating or preventing a CDI and/or a CDAD, comprising administering an effective amount of a pharmaceutical composition described herein to a subject or a patient need thereof. In various aspects, the CDI or CDAD comprises one or more of: C. difficile diarrhea (CDD), C. difficile intestinal inflammatory disease, colitis, pseudomembranous colitis, fever, abdominal pain, dehydration and disturbances in electrolytes, megacolon, peritonitis, and perforation and/or rupture of the colon.

In various aspects, a composition described herein is administered to a subject in need thereof to treat or prevent a disease or condition associated with GI dysbiosis in the context of initial onset or relapse/recurrence (e.g. due to continued or restarted antibiotic therapy). For example, in a subject that has previously suffered from a GI dysbiosis, the present pharmaceutical composition or formulation can be administered upon the first symptoms of recurrence in the subject. By way of non-limiting example, symptoms of recurrence include, in a mild case, about 5 to about 10 watery bowel movements per day, no significant fever, and only mild abdominal cramps while blood tests can show a mild rise in the white blood cell count up to about 15,000 (normal levels are up to about 10,000), and, in a severe case, more than about 12 watery stools per day, nausea, vomiting, high fever (e.g. about 102-104° F.), rectal bleeding, severe abdominal pain (e.g. with tenderness), abdominal distention, and a high white blood count (e.g. of about 15,000 to about 40,000).

In some aspects, the methods described herein can be used to treat a subject or patient who is suffering from, or is susceptible to, a disease or condition associated with GI dysbiosis. For example, the subject can be undergoing or have undergone an initial and/or adjunctive therapy that renders the subject susceptible to a disease or condition associated with GI dysbiosis. In some aspects, the subject is undergoing treatment, or has undergone treatment, with an antibiotic. For example, the subject can have taken an antibiotic during the past about 30 days and/or have an immune system that is weak (e.g. from a chronic illness). In another example, the patient can have recently been in the hospital, including in an intensive care unit. Accordingly, in some aspects, a method comprises administering a composition described herein to treat or prevent a nosocomial infection and/or a secondary emergent infection and/or a hospital acquired infection (HAI) in a subject in need thereof.

In various aspects, described herein are methods for treating antibiotic-induced adverse effects in the GI tract, comprising administering an effective amount of a microbial therapeutic (e.g., one or more bacterial isolates) to a subject in need thereof. In another aspect, provided herein are methods for preventing an antibiotic-induced adverse effect in the GI tract, comprising administering an effective amount of a microbial therapeutic to a subject in need thereof.

In another aspect, a pharmaceutical composition or a plurality of pharmaceutical compositions, as disclosed herein, can be used in the manufacture of a medicament, e.g., for treating a herein-described condition, disease, or disorder in a subject in need thereof. In various aspects, the bacterial isolates as described herein protect the intestinal microbiome from antibiotics-induced damage. In some aspects, the methods described herein can treat or prevent an antibiotics-associated adverse effect including but not limited to diarrhea, nausea, vomiting, dysgeusia, colitis, and pseudomembranous colitis disease and/or symptoms. In an aspect, methods described herein can be used to treat or prevent antibiotic-associated diarrhea (AAD).

Provided herein is a method of delivering one or more bile acid transforming bacteria to the intestine of a subject in need thereof, the method comprising administering to the subject a pharmaceutical composition comprising a bacterial mixture comprising one or more bile acid transforming bacteria, such that the one or more bile acid transforming bacteria engraft in the intestine of the subject and release a secondary bile acid or intermediate or derivative thereof into the subject's intestine. In an aspect, a secondary bile acid or intermediate or derivative thereof is taken up or absorbed by a cell of the host (e.g., a cell of the intestinal lining) where it used or metabolized by the host to alleviate a symptom of a disorder disclosed herein.

Provided herein is a method of delivering one or more bile acid transforming bacteria to the intestine of a subject in need thereof, the method comprising administering to the subject a bacterial mixture comprising a bacterial isolate and/or a consortium of fecal bacteria selected to produce one or more secondary bile acids, such that the bile acid transforming bacteria of the bacterial mixture engraft in the intestine of the subject and release the secondary bile acid or derivative or intermediate thereof into the subject's intestine. In an aspect, a relative abundance of one or more bile acid transforming bacteria in the intestine of the subject following administration of the pharmaceutical composition is greater than the relative abundance of the one or more bile acid transforming bacteria in the intestine of the subject prior to administration of the composition.

Provided herein is a method of delivering one or more bile acid transforming bacteria to the intestine of a subject in need thereof, the method comprising determining or diagnosing a level of a secondary bile acid produced by intestinal bacteria of the subject and administering to the subject a pharmaceutical composition based on the level of the secondary bile acid. In an aspect, the pharmaceutical composition is administered if the level of the secondary bile acid produced by the intestinal bacteria is below a threshold level.

In an aspect, a presence or level of secondary bile acid produced by the intestinal bacteria of the subject is determined directly from stool of the subject (e.g., in a direct secondary bile acid quantification assay using for example gas chromatography). In an aspect, a presence or level of secondary bile acid produced by the intestinal bacteria of the subject is determined using a TGR5 reporter cell assay as provided in the present disclosure. In another aspect, a presence or level of secondary bile acid produced by the intestinal bacteria of the subject is determined in a functional assay after extracting or harvesting the intestinal bacteria from stool of the subject (e.g., in an “ex vivo” assay that directly determines the capability of intestinal bacteria to produce one or more secondary bile acids). In another aspect, the functional assay includes a TGR5 reporter cell assay as provided in the present disclosure. In one aspect, the TGR5 reporter cell assay correlates the ability of an input substrate to activate the TGR5 receptor with a presence or level of secondary bile acid produced by the intestinal bacteria of the subject. In one aspect, the TGR5 reporter cell assay correlates the ability of an input substrate to activate the TGR5 receptor with a presence or level of bile acid transforming bacteria in the intestines of the subject. For example, fecal bacteria can be extracted from stool of a subject (e.g., by filtering and/or centrifuging), incubated for a period of time (e.g., at least 1 at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24 hours, or greater than 24 hours) with one or more primary bile acid or secondary bile acid substrates (e.g., one or more of the primary bile acids cholic acid or chenodeoxycholic acid or one or more of the secondary bile acids deoxycholic acid, isodeoxycholic acid, glycodeoxycholic acid, taurodeoxycholic acid, glycolithocholic acid, taurolithocholic acid, lithocholic acid, ursodeoxycholic acid, isoursodeoxycholic acid, glycoursodeoxycholic acid or tauroursodeoxycholic acid), and a rate of transformation of the bile acid substrate determined (e.g., by measuring directly or indirectly the reduction in the level of substrate or the amount of secondary bile acid produced).

In an aspect, a presence or level of secondary bile acid produced by the intestinal bacteria of a donor is determined directly from stool of the donor (e.g., in a direct secondary bile acid quantification assay using for example gas chromatography). In an aspect, a presence or level of secondary bile acid produced by the intestinal bacteria of the donor is determined using a TGR5 reporter cell assay as provided in the present disclosure. In another aspect, a presence or level of secondary bile acid produced by the intestinal bacteria of the donor is determined in a functional assay after extracting or harvesting the intestinal bacteria from stool of the donor (e.g., in an “ex vivo” assay that directly determines the capability of intestinal bacteria to produce one or more secondary bile acids). In another aspect, the functional assay includes a TGR5 reporter cell assay as provided in the present disclosure. In one aspect, the TGR5 reporter cell assay correlates the ability of an input substrate to activate the TGR5 receptor with a presence or level of secondary bile acid produced by the intestinal bacteria of the donor. In one aspect, the TGR5 reporter cell assay correlates the ability of an input substrate to activate the TGR5 receptor with a presence or level of bile acid transforming bacteria in the intestines of the donor. For example, fecal bacteria can be extracted from stool of a donor (e.g., by filtering and/or centrifuging), incubated for a period of time (e.g., at least 1 at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24 hours, or greater than 24 hours) with one or more primary bile acid or secondary bile acid substrates (e.g., one or more of the primary bile acids cholic acid or chenodeoxycholic acid or one or more of the secondary bile acids deoxycholic acid, isodeoxycholic acid, glycodeoxycholic acid, taurodeoxycholic acid, glycolithocholic acid, taurolithocholic acid, lithocholic acid, ursodeoxycholic acid, isoursodeoxycholic acid, glycoursodeoxycholic acid or tauroursodeoxycholic acid), and a rate or level of transformation of the bile acid substrate determined (e.g., by measuring directly or indirectly the reduction in the level of substrate or the amount of secondary bile acid produced).

In an aspect the at least one secondary bile acid is deoxycholic acid (DCA), and a subject is selected for treatment based on a level of DCA that is lower than a threshold value, for example in a stool of the subject or arising from a bile acid transforming functional assay incorporating fecal bacteria of the subject and a bile acid substrate, e.g., cholic acid. In an aspect, the at least one secondary bile acid is glycodeoxycholic acid (GDCA), and a subject is selected for treatment based on a level of GDCA that is lower than a threshold value, for example in a stool of the subject or arising from a bile acid transforming functional assay incorporating fecal bacteria of the subject and a bile acid substrate, e.g., DCA). In another aspect, the at least one secondary bile acid is taurodeoxycholic acid (TDCA), and a subject is selected for treatment based on a level of TDCA that is lower than a threshold value, for example in a stool of the subject or arising from a bile acid transforming functional assay incorporating fecal bacteria of the subject and a bile acid substrate, e.g., DCA. In another aspect, the at least one secondary bile acid is glycolithocholic acid (GLCA), and a subject is selected for treatment based on a level of GLCA that is lower than a threshold value, for example in a stool of the subject or arising from a bile acid transforming functional assay incorporating fecal bacteria of the subject and a bile acid substrate, e.g., LCA). In another aspect, the at least one secondary bile acid is lithocholic acid (LCA), and a subject is selected for treatment based on a level of LCA, for example in a stool of the subject or arising from a bile acid transforming functional assay incorporating fecal bacteria of the subject and a bile acid substrate, e.g., chenodeoxycholic acid. In another aspect, the at least one secondary bile acid is ursodeoxycholic acid (UDCA), and a subject is selected for treatment based on a level of UDCA, for example in a stool of the subject or arising from a bile acid transforming functional assay incorporating fecal bacteria of the subject and a bile acid substrate, e.g., LCA). In another aspect, the at least one secondary bile acid is glycoursodeoxycholic acid (GUDCA), and a subject is selected for treatment based on a level of GUDCA, for example in a stool of the subject or arising from a bile acid transforming functional assay incorporating fecal bacteria of the subject and a bile acid substrate, e.g., UDCA). In a still further aspect, the at least one secondary bile acid is tauroursodeoxycholic acid (TUDCA), and a subject is selected for treatment based on a level of TUDCA, for example in a stool of the subject or arising from a bile acid transforming functional assay incorporating fecal bacteria of the subject and a bile acid substrate, e.g., UDCA).

In an aspect, a pharmaceutical composition described herein can be administered to the subject based on a determination that the secondary bile acid produced by the fecal bacteria (e.g., directly from a stool or in a functional assay such as an ex vivo assay) is below a threshold level. For example, the threshold level can be 5 μM, 10 μM, 15 μM, 20 μM, 30 μM, 40 μM, 50 μM, 60 μM, 70 μM, 80 μM, 90 μM, 100 μM, 120 μM, 140 μM, 150 μM, 170 μM, 200 μM, 300 μM, 350 μM, 400 μM, 450 μM, or 500 μM. In another aspect, the threshold level is between 5 μM and 20 μM, 5 μM and 50 μM, 5 μM and 100 μM, and 5 μM and 150 μM, 5 μM and 200 μM, 5 μM and 800 μM, 10 μM and 20 μM, 10 μM and 50 μM, 10 μM and 100 μM, 10 μM and 150 μM, 10 μM and 200 μM, 50 μM and 100 μM, 50 μM and 200 μM, 100 μM and 200 μM, 200 μM and 400 μM, 200 μM and 600 μM, 400 μM and 800 μM, or 200 μM and 800 μM.

Herein a “subject in need thereof” can have a disease, disorder or condition disclosed herein, the treatment or alleviation of which can be facilitated by the administration of one or more bacterial species capable of transforming a primary bile acid to a secondary bile acid or converting between secondary bile acids. In an aspect, a subject is in need of treatment on the basis of the selection of such subject as a candidate who may benefit from supplementation with bile acid transforming bacteria by applying the methods disclosed herein. For example, a subject may be in need of treatment on the basis that a bile acid metabolism consensus sequence is absent in a microbiota of the subject or present in a number of bacterial clades that is below a threshold number, or if a level or rate of production of a secondary bile acid produced by fecal bacteria of the subject in a functional assay or present in stool of the subject is less than a threshold value.

In an aspect of the present disclosure, administration of a pharmaceutical composition to a subject in need results in one or more of (i) an increase in the absolute level or the concentration of one or more secondary bile acids in the intestine of the subject, or (ii) an increase in the secondary bile acid to primary bile acid ratio, without changing the overall abundance of bile acids, or (iii) an increase in the relative abundance of primary bile acid transforming bacteria compared to such relative abundance prior to treatment, or (iv) an increase in the relative abundance of secondary bile acid converting bacteria compared to such relative abundance prior to treatment, or (v) an increase in the level of expression of one or more of the genes FXR, PXR, LXR, TGR5 or ROR, compared to expression prior to treatment.

In a further aspect, the selection of a subject for treatment can vary depending on the presence, abundance or status of a characteristic or biomarker of the subject. Non-limiting examples of such characteristics or biomarkers include age, sex, medical history, comorbidity or history of drug or antibiotic administration, a level of gene expression of a host cell (e.g., FXR, PXR, LXR, TGR5 and ROR), the presence or abundance of a metabolite, a relative abundance of a non-secondary bile acid producing bacterial taxa in the fecal microbiota of the subject, and the abundance of a microbial or metabolic marker of an intestinal dysbiosis. Non-limiting examples of a marker of an intestinal dysbiosis include an abundance of the metabolite calprotectin, breath methane to hydrogen ratio, intestinal inflammation, Firmicutes/Bacteroidetes ratio, fecal microbial diversity, gut permeability, and fecal short-chain fatty acid (SCFA) level. In another aspect, a subject in need of a treatment comprises a reduced relative abundance of a bacterial taxa selected from the group consisting of Firmicutes, Bacteroidetes, Actinobacteria, Bacteroides, Faecalihacterium, Clostridium, Ruminococcus, Coprococcus, Eubacterium, Dorea, Blautia, Roseburia, Butyrivibrio, Subdoligramium, and Anaertostipes. In another aspect, a subject in need of a treatment comprises a reduced relative abundance of a bacterial taxa selected from the group consisting of Bacteroides, Parabacteroides, Butyricimonas, Odoribacter, Porphyromonas, Alistipes, and a combination thereof. In an aspect a subject in need of treatment comprises a lower ratio of secondary bile acids to primary bile acids compared to a healthy human donor, or a reduced abundance or concentration of secondary bile acids compared to a healthy human donor.

In an aspect, a subject administered a pharmaceutical composition (e.g., comprising a bacterial mixture) can be monitored and/or tested for remediation or alleviation of a bile acid deficiency following the administration. For example, metagenomic sequence data generated from the subject's microbiota following treatment can be analyzed for the presence of bacterial clades having a bile acid metabolism consensus sequence, and the number of bacterial clades having the consensus sequence compared to such number of bacterial clades prior to treatment. Alternatively or in addition, a level or presence of one or more secondary bile acids can be quantified directly from a stool of the subject, or a level or presence of one or more secondary bile acids produced by the fecal bacteria in a functional assay (e.g., an in vivo assay) can be determined. In various aspects, such analyses to verify or test the efficacy of a treatment in alleviating a deficiency in secondary bile acid metabolism can be performed at least 6 hours, at least 12 hours, at least 18 hours, at least 24 hours, at least 1 day, at least 2 days, at least 3 days, at least 4 days, at least 5 days, at least 6 days, at least 7 days, at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, or at least 16 weeks following administration of a pharmaceutical composition.

In an aspect, a method comprises determining using the methods provided herein that a subject has a deficiency in bile acid metabolism, administering a first dose of a pharmaceutical composition described herein based on such determination, determining again that the subject continues to have a deficiency in bile acid metabolism following administration, and administering a second dose of the pharmaceutical composition based on such second determination. In an aspect, the first dose is equal to or lower than the second dose. In another aspect, the second dose is equal to or lower than the first dose.

In one aspect of the present disclosure, methods provide for treating a subject in need thereof comprising: (1) administering to the subject a first pharmaceutically active dose of a pharmaceutical composition comprising a consortium of fecal bacteria from a single donor; (2) testing of the subject to determine efficacy, if an additional dose is necessary, or if the dose should be adjusted; (3) administration of a second pharmaceutical composition comprising a consortium of fecal bacteria blended from multiple donors; (4) optionally testing of the subject to determine efficacy, if an additional dose is necessary, or if the dose should be adjusted; and (5) optionally administrating a third pharmaceutical composition comprising a consortium of fecal bacteria blended from multiple donors, where the multiple donors (a) comprise all donors used to produce the second pharmaceutical composition and additional donors, (b) comprise donors not used to produce the second pharmaceutical composition, (c) comprise some but not all of the donors used to produce the second pharmaceutical composition, or (d) comprise only donors not used to produce the second pharmaceutical composition. In another aspect, the first, second, and third pharmaceutical compositions are administered in a different order (i.e., first, third, second; third, second, first; third, first, second; second, first, third, etc.).

Methods for measuring change and/or improvement in GI tract function can include, but are not limited to: endoscopy for direct examination of epithelium and mucosa; histological evaluation and/or tissue procurement for direct evaluation of structural changes and/or immune biomarkers; urine tests for assessment of permeability with non-absorbable sugars and LPS levels; stool tests for assessment of inflammation and/or microbiota changes (for example by PCR); and/or blood tests for assessment of specific markers, including CD4+ cell counts, Th17 cell counts, and/or LPS levels.

In another aspect, disclosed herein are a plurality of pharmaceutical compositions, e.g., two or more pharmaceutical compositions, as disclosed herein, for use in the prevention or treatment of a condition, disease, or disorder in a subject in need thereof. In aspects, a first composition comprises one or more bacterial isolates described herein. In aspects, a second composition comprises a consortium of fecal bacteria (e.g., a consortium of fecal bacteria or a fermented fecal microbiota). A subject can be treated with the first and second compositions in any order to treat or prevent a disorder. For example, in one aspect a subject is treated with a composition comprising a consortium of fecal bacteria, followed by a composition comprising one or more bacterial isolates. In another aspect, a subject is treated with a composition comprising one or more bacterial isolates followed by a composition comprising a consortium of fecal bacteria. In still other aspects, a subject can be treated with a composition comprising one or more bacterial isolates and a composition comprising a consortium of fecal bacteria simultaneously (for example, with a composition comprising both the bacterial isolate(s) and the consortium, or with multiple compositions each comprising one of the bacterial isolate(s) or a consortium of fecal bacteria).

In an aspect, a method for treating or preventing a condition, disease or disorder of a subject comprises administration to the subject of: (i) a pharmaceutical composition comprising one or more bacterial isolates; and (ii) a consortium of fecal bacteria. For example, the one or more bacterial isolates can be administered before or after the consortium of fecal bacteria, or at the same time (e.g., in different compositions or together in the same composition). In another aspect, a method for treating a disorder of a subject comprises administration to the subject of: (i) a pharmaceutical composition comprising a bacterial mixture (e.g., comprising a consortium of fecal bacteria); and (ii) one or more antibiotics. Typically, the antibiotic is administered to the subject prior to administration of the bacterial mixture, in order to purge the subject's intestine of harmful and/or pathogenic bacteria prior to replenishment of the gut with bacteria from the bacterial mixture. In an aspect, a method for treating a disorder of a subject comprises administration to the subject of: (i) a pharmaceutical composition comprising a bacterial mixture (e.g., comprising a consortium of fecal bacteria); and (ii) a prebiotic. For example, the bacterial mixture can be administered before or after the prebiotic, or at the same time (e.g., in different compositions or together in the same composition). For each of the above examples, it is further understood that any given component in a method of treatment can be administered multiple times. For example, a consortium of fecal bacteria can be administered to the subject, followed by one or more bacterial isolates, followed by a second administration of a consortium of fecal bacteria.

In an aspect, a method for treating or preventing a condition, disease or disorder of a subject comprises administration to the subject of: (i) a pharmaceutical composition comprising one or more bacterial isolates; (ii) a consortium of fecal bacteria; and (iii) one or more antibiotics. The different components of (i)-(iii) can be administered to the subject in any order. For example, a subject can be administered one or more antibiotics, followed by a consortium of fecal bacteria, followed by one or more bacterial isolates. In another example, the subject can be administered one or more antibiotics, followed by one or more bacterial isolates, followed by a consortium of fecal bacteria. For each of the above examples, it is further understood that any given component in a method of treatment can be administered multiple times. For example, an antibiotic can be administered to the subject, followed by a consortium of fecal bacteria, followed by one or more bacterial isolates, followed by a second administration of a consortium of fecal bacteria.

In an aspect, a method for treating or preventing a condition, disease or disorder of a subject comprises administration to the subject of: (i) a pharmaceutical composition comprising one or more bacterial isolates; (ii) a consortium of fecal bacteria; and (iii) one or more prebiotics. The different components of (i)-(iii) can be administered to the subject in any order. For example, a subject can be administered one or more prebiotics, followed by a consortium of fecal bacteria, followed by one or more bacterial isolates. In another example, the subject can be administered one or more prebiotics, followed by one or more bacterial isolates, followed by a consortium of fecal bacteria. In another example, the subject can be administered one or more prebiotics following administration of one or both of the one or more bacterial isolates and/or a consortium of fecal bacteria. For each of the above examples, it is further understood that any given component in a method of treatment can be administered multiple times. For example, a consortium of fecal bacteria can be administered to a subject, followed by a one or more bacterial isolates, followed by a prebiotic, followed by a second administration of a consortium of fecal bacteria.

In an aspect, a method for treating or preventing a condition, disease or disorder of a subject comprises administration to the subject of: (i) a pharmaceutical composition comprising one or more bacterial isolates; (ii) a consortium of fecal bacteria; (iii) one or more prebiotics; and (iv) one or more antibiotics. The different components of (i)-(iv) can be administered to the subject in any order. For example, a subject can be administered one or more antibiotics, followed by one or more prebiotics, followed by a consortium of fecal bacteria, followed by one or more bacterial isolates. In another example, the subject can be administered one or more antibiotics, followed by one or more prebiotics, followed by one or more bacterial isolates, followed by a consortium of fecal bacteria. In another example, the prebiotic can be administered after one or both of the one or more bacterial isolates and/or the consortium of fecal bacteria. For each of the above examples, it is further understood that any given component in a method of treatment can be administered multiple times. For example, an antibiotic can be administered to a subject, followed by a consortium of fecal bacteria, followed by one or more bacterial isolates, followed by a prebiotic, followed by a second administration of a consortium of fecal bacteria.

In each of the above combination treatments, the duration of time between different treatments (e.g., between administration of a consortium of fecal bacteria and one or more bacterial isolates) can be at least 1 hour, at least 2 hours, at least 6 hours, at least 12 hours, at least 1 day, at least 2 days, at least 3 days, at least 4 days, at least 5 days, at least 6 days, at least 1 week, at least 2 weeks, at least 3 weeks, at least 4 weeks, at least 5 weeks, at least 6 weeks, at least 7 weeks, at least 8 weeks, or greater than 8 weeks.

In one aspect, a subject being treated by administering a composition described herein is a subject already with a disorder (e.g., IBD, ulcerative colitis, Crohn's disease, ASD, hepatitis B or hepatic encephalopathy). In another aspect, a pharmaceutical composition is administered to a clinically asymptomatic human subject who is genetically predisposed or prone to a disorder (e.g., IBD, ulcerative colitis, Crohn's disease, ASD, hepatitis B or hepatic encephalopathy) is also useful in preventing the onset of clinical symptoms. A human subject genetically predisposed or prone to a disorder can be a human subject having a close family member or relative exhibiting or having suffered the disorder. In another aspect, a subject being treated by administering a composition described herein is a subject in which a disorder is to be prevented. In another aspect, a subject being treated by administering a composition described herein is predisposed or susceptible to a disorder. In another aspect, a subject being treated by administering a composition described herein is a subject diagnosed as having a disorder. In one aspect, a subject being treated by administering a composition described herein is a patient in need thereof.

In one aspect, the present disclosure provides a method for treating a subject in need thereof, where the method comprises administering to the subject multiple pharmaceutically active doses of a pharmaceutical composition comprising a bacterial mixture, such that different doses comprise a consortium of fecal bacteria derived from different carefully screened, healthy donors. In an aspect, a subject is administered a pharmaceutical composition over a dosing period wherein a first dose comprises at least one pharmaceutical composition comprising a consortium of fecal bacteria of a single donor, and a second dose of a pharmaceutical composition comprises a consortium of fecal bacteria of a single donor different from the donor of the first dose. In another aspect, a first dose comprises a pharmaceutical composition comprising a consortium of fecal bacteria of a single donor and a second dose comprises a consortium of fecal bacteria of a donor pool. The first and second dose do not indicate the order of administration to a subject, but rather that the consortium of fecal bacteria from different donors may be used in a non-blended form. In yet another aspect, the consortium of fecal bacteria from multiple carefully screened, healthy donors is provided in a blended form.

In an aspect, a pharmaceutical composition used herein comprises a bacterial mixture comprising a consortium of fecal bacteria derived from a donor with preselected desirable characteristics or receiving certain pre-treatment(s). In an aspect, a donor has no current or previous diagnosis or has no symptom of a disorder (e.g., IBD, ulcerative colitis, Crohn's disease, ASD, hepatitis B or hepatic encephalopathy) to be treated with a consortium of fecal bacteria derived from the donor. In another aspect, a donor has no family member or direct relative diagnosed with a disorder or exhibiting a symptom of a disorder to be treated with a consortium of fecal bacteria derived from the donor. In another aspect, a donor has no siblings, parents, or children diagnosed with or exhibiting a symptom of a disorder to be treated with a consortium of fecal bacteria derived from the donor. In an aspect, a donor has not previously received any fecal microbiota transplantation. In an aspect, a fecal donor previously donated a stool for treating a GI disorder, e.g., a C. difficile infection or IBD.

### Manufacture of Compositions

In a further aspect, provided herein is use of a bacterial mixture described herein for manufacture of a medicament for alleviating or rescuing a deficiency in bile acid metabolism, for treating a disorder described herein or for reducing the severity of one or more symptoms of a disorder described herein.

Provided herein are pharmaceutical compositions comprising fecal bacteria (e.g., a consortium of fecal bacteria or one or more bacterial isolates) selected to produce one or more secondary bile acids in an intestine of a subject administered the composition. In aspects, a method of manufacturing a composition comprises selecting fecal bacteria of stool of a donor on the basis of any one of the following: (i) determining that the fecal bacteria comprise one or more clades of bile acid producing bacteria, e.g., by identifying one or more bile acid metabolism consensus sequences in the fecal bacteria, (ii) determining that the relative abundance of fecal bacteria comprising one or more clades of bile acid producing bacteria is above a threshold value, or (iii) determining that the fecal bacteria are capable of producing one or more secondary bile acids at or above a threshold level, for example by quantifying a secondary bile acid from stool of the donor or via an ex vivo assay. For example, in the case of an ex vivo assay, where fecal bacteria extracted from stool of a donor produce one or more secondary bile acids at or above a threshold level, then fecal bacteria from stool of the donor (either the same stool used to extract bacteria for the assay or a different stool) can be selected for incorporation into the bacterial mixture.

The present disclosure contemplates that any method, assay, test or protocol described herein to select a subject for treatment can also be used to select a donor, or stool or a consortium of fecal bacteria of a donor comprising bacteria capable of producing one or more secondary bile acids when administered to a subject. For example, in an aspect, a method comprises determining the presence or an amount of one or more secondary bile acids in stool of a donor (i.e., harboring fecal bacteria), extracting fecal bacteria from stool of the donor (e.g., as a consortium of fecal bacteria extracted from the same stool or a different stool) to produce a consortium of fecal bacteria, and incorporating the fecal bacteria into the pharmaceutical composition based on the presence or the amount of the one or more secondary bile acids in the stool. In another aspect, a method comprises extracting fecal bacteria from stool of a donor, directly determining an ability or potential of the fecal bacteria to produce one or more secondary bile acids (e.g., at or above a threshold level), and incorporating the fecal bacteria (e.g., as a consortium of fecal bacteria extracted from the same stool or a different stool) into a pharmaceutical composition based on the ability of the fecal bacteria to produce the one or more secondary bile acids. In another aspect, a method comprises determining a quantity or relative abundance of one or more clades of bile acid transforming bacteria from stool of a donor, and incorporating fecal bacteria from stool of the donor (e.g., as a consortium of fecal bacteria extracted from the same stool or a different stool) into a pharmaceutical composition based on the quantity or relative abundance of the bacterial clades.

In an aspect, fecal bacteria of a stool of a donor can be incorporated into a pharmaceutical composition on the basis that the number of clades of bacteria comprising a bile acid metabolism consensus sequence is at or above a threshold value. In an aspect, the fecal bacteria are selected if the number of clades of bacteria is at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, or at least twenty. The number of clades of bile acid transforming bacteria in a consortium of fecal bacteria of a donor can be determined by identifying the number of clades of bacteria having a bile acid metabolism consensus sequence using metagenomics sequencing data derived from a consortium of fecal bacteria of a donor. In an aspect, the bile acid metabolism consensus sequence comprises or is indicative of or adjacent to one or more bai genes or a bai operon. In certain instances, fecal bacteria can be selected when the relative abundance of one or more bile acid transforming bacterial clades is at or above a threshold value.

Any method known in the art can be used to determine or quantify an amount of a secondary bile acid from a stool.

In various aspects, fecal bacteria of stool of a donor are selected for incorporation into a pharmaceutical composition on the basis of the presence or abundance of a secondary bile acid, wherein the secondary bile acid is selected from the group consisting of deoxycholic acid (DCA), isodeoxycholic acid, glycodeoxycholic acid (GDCA), taurodeoxycholic acid (TDCA), glycolithocholic acid (GLCA), taurolithocholic acid (TLCA), lithocholic acid (LCA), isoalloLCA, ursodeoxycholic acid (UDCA), isoursodeoxycholic acid, glycoursodeoxycholic acid (GUDCA), tauroursodeoxycholic acid (TUDCA) and a combination thereof. In various aspects, fecal bacteria of stool of a donor are selected for incorporation into a pharmaceutical composition on the basis of the presence or abundance of a secondary bile acid, wherein the secondary bile acid is isoalloLCA. In aspects, the fecal bacteria can be incorporated on the basis that the one or more secondary bile acids are present in the stool at or above a threshold level. For example, the threshold level can be 200 μM, 250 μM 300 μM, 350 μM, 400 μM, 450 μM, 500 μM, 550 μM, 600 uM, 650 μM, 700 μM, 750 μM, 800 μM, 850 μM, 900 μM, 950 μM, 1000 μM, 1100 μM, 1200 μM, 1300 μM, 1400 μM, 1500 μM, 1600 μM, 1700 μM, 1800 μM, 1900 μM, 2000 μM or greater than 2000 μM.

In an aspect, a method comprises directly determining an ability or potential of fecal bacteria from stool of a donor to produce one or more secondary bile acids (e.g., at or above a threshold level), and incorporating a preparation of uncultured fecal bacteria prepared from stool of the same donor (e.g., the same stool or a different stool) into a pharmaceutical composition based on the ability or potential of the fecal bacteria to produce the one or more secondary bile acids. In this aspect, intestinal bacteria are screened or assayed in real time for their ability to produce a secondary bile acid by metabolizing one or more substrates during an incubation period. Any functional assay or ex vivo assay known to a person skilled in the art can be used to assay the bacteria for their ability to produce the bile acid. Typically in such functional assays the fecal bacteria are at least partially purified, extracted or harvested from non-bacterial stool matter (e.g., fiber) prior to incubating the bacteria with a substrate. This method for screening fecal bacteria for an ability to produce a secondary bile acid is advantageous in that it identifies a source of fecal bacteria (e.g., the stool from which a sample of bacteria is extracted) known to be viable and functionally capable of generating a secondary bile acid. Bacterial cells from a remaining portion of the stool (i.e., not used in performing the assay), or from a different stool of the same donor, can then be incorporated into a bacterial mixture for incorporation into a pharmaceutical composition. In a non-limiting example of an ex vivo assay, a sample of raw stool from a donor (e.g., about 10 ml) can be homogenized and centrifuged, the pellet resuspended in buffer (e.g., sodium phosphate buffer), and the fecal bacteria incubated with one or more primary or secondary bile acid substrates, as described above.

In an aspect, a functional assay comprises exposing fecal bacteria of a donor (e.g., a consortium of fecal bacteria) to a primary bile acid for a period of time (e.g., at least 1 at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24 at least 30, at least 36 hours, at least 42 hours, at least 48, at least 54, at least 60, or greater than 60 hours) and determining the rate of conversion of the primary bile acid to its corresponding bile acid. In an aspect, the primary bile acid is CA and the secondary bile acid is DCA. In another aspect, the primary bile acid is CDCA and the secondary bile acid is LCA. In an aspect the secondary bile acid is isoalloLCA. In various aspects, a determination of the rate of conversion or transformation comprises (i) comparing the level of primary bile acid substrate at the beginning and end of the assay, (ii) determining a level of secondary bile acid at the end of the assay, and/or comparing a ratio of the secondary bile acid to the primary bile acid at the beginning and end of the assay. In an aspect, a donor or stool from a donor can be selected on the basis that a ratio of secondary to primary bile acids existing at the end of the functional assay (e.g., during a period of less than 24 hours or less than 12 hours) is greater than 1. In another aspect, a subject can benefit from treatment or can be selected for treatment on the basis that a ratio of secondary to primary bile acids existing at the end of the functional assay (e.g., during a period of less than 24 hours or less than 12 hours) is greater than 0.5. In another aspect, a subject can benefit from treatment or can be selected for treatment on the basis that a ratio of secondary to primary bile acids existing at the end of the functional assay (e.g., during a period of less than 24 hours or less than 12 hours) is greater than 3, greater than 2.5, greater than 2, greater than 1.9, greater than 1.8, greater than 1.7, greater than 1.6, greater than 1.5, greater than 1.4, greater than 1.3, greater than 1.2, greater than 1.1, greater than 1, greater than 0.9, greater than 0.8, greater than 0.7, greater than 0.6, or greater than 0.5.

In an aspect, a subject is selected as potentially benefiting from administration with a composition described herein, or for treatment with a composition described herein, based on determining that a level or concentration of at least one secondary bile acid in a stool of the subject or arising from a functional assay (or a ratio of a secondary to primary bile acid in a stool or arising from a functional assay) is greater than a threshold level, wherein the secondary bile acid is selected from the group consisting of deoxycholic acid (DCA), isodeoxycholic acid, glycodeoxycholic acid (GDCA), taurodeoxycholic acid (TDCA), glycolithocholic acid (GLCA), taurolithocholic acid (TLCA), lithocholic acid (LCA), isoalloLCA, ursodeoxycholic acid (UDCA), isoursodeoxycholic acid, glycoursodeoxycholic acid (GUDCA), tauroursodeoxycholic acid (TUDCA) or a combination thereof.

In an aspect, a donor or a stool or a donor is selected for manufacturing a composition described herein on the basis of a ratio of total secondary bile acids to total primary bile acids in a stool being greater than a threshold value. In an aspect, the subject is selected if the ratio of total secondary bile acids to total primary bile acids is greater than 3, greater than 2.5, greater than 2, greater than 1.9, greater than 1.8, greater than 1.7, greater than 1.6, greater than 1.5, greater than 1.4, greater than 1.3, greater than 1.2, greater than 1.1, greater than 1, greater than 0.9, greater than 0.8, greater than 0.7, greater than 0.6, or greater than 0.5.

In another aspect, a donor or a stool of a donor is selected if the ratio of a secondary bile acid to its corresponding primary bile acid in a stool of the subject is greater than a threshold value. In an aspect, the ratio of secondary bile acid to primary bile acid is selected from the group consisting of DCA:CA, LCA:CDCA, 12-oxoLCA:CA, 3-oxoCA:CA, 7-oxoDCA:CA, 3-oxoLCA:CDCA, 7-oxoLCA:CDCA, and a combination thereof. In an aspect, a donor or stool of a donor is selected if the ratio of the secondary bile acid to its corresponding primary bile acid in a stool of the subject is greater than 3, greater than 2.5, greater than 2, greater than 1.9, greater than 1.8, greater than 1.7, greater than 1.6, greater than 1.5, greater than 1.4, greater than 1.3, greater than 1.2, greater than 1.1, greater than 1, greater than 0.9, greater than 0.8, greater than 0.7, greater than 0.6, or greater than 0.5.

In a further aspect, a donor or a stool of a donor is selected if the ratio of a second secondary bile acid to a first secondary bile acid in a stool of the subject is greater than a threshold value. In an aspect, the ratio of second secondary bile acid to first secondary bile acid is selected from the group consisting of IDCA:DCA, GDCA:DCA, TDCA:DCA, GLCA:LCA, TLCA:LCA, UDCA:LCA, isoLCA:3-oxoLCA, isoalloLCA:3-oxoLCA, 3-oxoalloLCA:3-oxoLCA, or a combination thereof. In an aspect, the subject is selected if the ratio of the secondary bile acid to its corresponding primary bile acid in a stool of the subject is greater than 3, greater than 2.5, greater than 2, greater than 1.9, greater than 1.8, greater than 1.7, greater than 1.6, greater than 1.5, greater than 1.4, greater than 1.3, greater than 1.2, greater than 1.1, greater than 1, greater than 0.9, greater than 0.8, greater than 0.7, greater than 0.6, or greater than 0.5.

In various aspects, fecal bacteria of stool of a donor are selected for incorporation into a pharmaceutical composition on the basis of a determination (e.g., using a functional assay) that fecal bacteria of stool of the donor are capable of producing a secondary bile acid at or above a threshold level. In an aspect, the threshold level. For example, the threshold level can be 200 μM, 250 μM 300 μM, 350 μM, 400 μM, 450 μM, 500 μM, 550 μM, 600 uM, 650 μM, 700 μM, 750 μM, 800 μM, 850 μM, 900 μM, 950 μM, 1000 μM, 1100 μM, 1200 μM, 1300 μM, 1400 μM, 1500 μM, 1600 μM, 1700 μM, 1800 μM, 1900 μM, 2000 μM or greater than 2000 μM of the secondary bile acid.

In an aspect, a bacterial mixture is prepared from the same stool that is assayed to determine a suitability of fecal bacteria of the stool to be incorporated into a composition (e.g., directly by quantification of the secondary bile acids in the stool, via a functional assay that determines the level of secondary bile acids produced by fecal bacteria from the stool, or by determining a quantity or relative abundance of one or more clades of bile acid transforming bacteria). For example, a stool received from a donor can be divided into two or more portions, of which a first portion can be subjected to an assay for determining an ability of fecal bacteria of the stool to produce one or more bile acids, and a second portion (e.g., the remaining stool) can be stored and/or frozen until the determination is made. If the fecal bacteria of the first portion are determined to be capable of producing one or more secondary bile acids (e.g., by determining a level of the one or more secondary bile acids in the first portion to be at or above a threshold level, by determining a level of the one or more secondary bile acids produced by the fecal bacteria in a functional assay to be at or above a threshold level, or by determining a quantity or relative abundance of one or more clades or file acid transforming bacteria in the first portion to be at or above a threshold level), then the stored stool can be processed to produce a bacterial mixture for incorporation into a pharmaceutical composition. In another aspect, a bacterial mixture is prepared from fecal bacteria from a stool of the same donor, but a different stool than was used to determine the suitability of the fecal bacteria for incorporation into the pharmaceutical composition.

Disclosed herein is a method of selecting a stool donor comprising determining that fecal bacteria of stool of the donor can produce one or more secondary bile acids (e.g., at or above a threshold level), selecting stool of the donor (e.g., the same stool or different stool) based on the determination that the fecal bacteria can produce one or more secondary bile acids, and incorporating fecal bacteria of the selected stool into a pharmaceutical composition (e.g., as a consortium of fecal bacteria).

In an aspect, a method of selecting a human stool donor (or a method of selecting stool of a donor for producing a bacterial mixture) comprises determining that fecal bacteria of a plurality of stools of the donor can produce one or more secondary bile acids, for example by determining that a secondary bile acid is produced at or above a threshold level (e.g., in an ex vivo assay or by directly quantifying from stool) in multiple stools or by determining that a quantity or relative abundance of one or clades of bile acid transforming bacteria in multiple stools is at or above threshold level. In aspects, stool from the donor can be collected at different time points, such as 2, 3, 4, 5, 6, 7, 8, 9, 10, 11, 12, 13, 14, 15, 16, 17, 18, 19, 20, or greater than 20 time points, and the ability of fecal bacteria in each stool to produce one or more secondary bile acids determined at each time point. In an aspect, the plurality of stools collected from the donor are from successive bowel movements of the donor. In an aspect, the plurality of stools collected from the donor are collected on the same day. In an aspect, the plurality of stools collected from the donor are collected on different days. In an aspect, at least two of the plurality of stools collected from the donor are collected at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or greater than 24 hours apart. In an aspect, at least two of the plurality of stools collected from the donor are collected at least 1, at least 2, at least 3, at least 4, at least 5, at least 6, at least 7, or greater than 7 days apart. In an aspect, at least two of the plurality of stools collected from the donor are collected at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or greater than 20 weeks apart. In an aspect, at least two of the plurality of stools collected from the donor are collected at least 1, at least 2, at least 3, at least 4, at least 5, at least 6, at least 7, at least 8, at least 9, at least 10, at least 11, at least 12, or greater than 12 months apart.

Provided herein is a method of selecting a stool donor (or a method of selecting stool of a donor for producing a bacterial mixture) comprising determining that fecal bacteria from a first stool of an individual can produce one or more secondary bile acids, determining that fecal bacteria of a second stool of the individual (e.g., collected at a different time point) can produce one or more bile acids, and selecting the individual as the stool donor based on determining that fecal bacteria from the first and the second stools produce the one or more bile acids. The method can further comprise extracting fecal bacteria from stool of the selected donor for incorporating into a pharmaceutical composition (e.g., as a consortium of fecal bacteria).

In an aspect, the donor is selected based on determining from at least 2, at least 3, at least 4, at least 5, at least 6, at least 7, at least 8 at least 9, at least 10, at least 11, at least 12, at least 13, at least 14, at least 15, or greater than 15 stools that fecal bacteria of each stool can produce one or more bile acids. In an aspect, once a stool donor is selected, stool of the donor can be collected and fecal bacteria from the stool used to produce a consortium of fecal bacteria for incorporation into a pharmaceutical composition described herein.

In another aspect, prior to making a fecal donation, a stool donor can ingest one or more bacterial isolates (e.g., one or more bile acid transforming bacteria), for example in the form of one or more probiotics. In an aspect, a stool donor can ingest a bacterial isolate prior to donating a stool in order to introduce the bacterial isolate into fecal bacteria of the donated stool. Therefore, a bacterial isolate desirable for inclusion in a bacterial mixture of a pharmaceutical composition can be introduced into the fecal bacteria of a stool donor via ingestion of the bacterial isolate by the donor, thereby allowing a consortium of fecal bacteria to be prepared from the stool of the donor that is “ready-made” with, or already includes, a species or strain comprising bile acid transforming bacteria. A consortium of fecal bacteria prepared from the stool of a donor that has ingested a bacterial isolate (e.g., in the form of a probiotic) can be directly incorporated into a pharmaceutical composition described herein, without adding any additional bacterial isolate to the preparation, or alternatively can be further spiked or enriched with an additional dose of the bacterial isolate. Such “pre-spiking” of a stool donor's fecal microbiota with one or more desired bacterial strain or species originating as bacterial isolates dosed to a stool donor can be especially advantageous where a fecal microbiota of the donor does not endogenously comprise bacteria of the same taxonomic category as the bacterial isolate (e.g., phylum, class, order, family, genus, species, or strain), or does not endogenously comprise a bacterial strain or species having a genetic identity to the bacterial isolate that is above a threshold level (e.g., having a 16S rRNA sequence with greater than 97% identity, greater than 98% identity, greater than 99% identity, greater than 99.1% identity, greater than 99.2% identity, greater than 99.3% identity, greater than 99.4% identity, greater than 99.5% identity, greater than 99.6% identity, greater than 99.7% identity, greater than 99.8% identity, or greater than 99.9% identity to a 16S rRNA sequence of the bacterial isolate). In aspects, the probiotic or bacterial isolate comprises one or more of Clostridium, Bacillus, Bacteroides, Bifidobacterium, Catenibacterium, Collinsella, Eggerthella, Lachnospira, Lactobacillus, Parabacteroides, Peptoniphilus, Mediterraneibacter, Clostridium hiranonis, Clostridium hylemonae, Clostridium scindens, Ruminococcus gnavus, Coprococcus comes, Faecalibacterium prausnitzii, Eubacterium ventriosum, Bacteroides pectinophihs, Dorea formicigenerans, Eggerthela lenta, Parabacteroides merdae, Bacteroides dorei, Bacteroides vulgatus, and a combination thereof. In an aspect, the probiotic or bacterial isolate comprises one or more of Bacteroides, Parabacteroides, Butyricimonas, Odoribacter, Porphyromonas, Alistipes, and a combination thereof. In an aspect, the probiotic or bacterial isolate comprises one or more of Bacteroides uniformis, Bacteroides finegoldii, Bacteroides fragilis, Bacteroides dorei, Bacteroides vulgatus, Bacteroides caccae, Bacteroides ovatus, Bacteroides eggerthii, Bacteroides thetaiotaomicron, Parabacteroides goldsteinii, Parabacteroides distasonis, Parabacteroides merdae, Butyricimonas synergistica, Odoribacter laneus, Porphyromonas somerae, Alistipes indistinctus, Alistipes onderdonkii, Alistipes finegoldii, and a combination thereof.

In an aspect, a donor can ingest a probiotic comprising a bacterial isolate comprising bile acid transforming bacteria of a taxonomic category that is not detectable, or is present at a relative abundance below a threshold abundance, in a stool of the donor prior to ingestion of the probiotic. For example, the microbiota of a stool of a donor can be screened (e.g. using a nucleic acid hybridization technique such as PCR) for the presence of a particular taxa (phylum, class, order, family, genus, species, or strain) in the fecal microbiota of the donor. If the taxa either is not found in the fecal microbiota, or is present at a relative abundance below a threshold abundance, then the donor can be administered or ingest a probiotic comprising a bacterial isolate of that taxa.

In an aspect, a duration of time between ingestion of one or more bacterial isolates by a stool donor and collection of a stool from the donor (i.e., comprising a bacterial species originating from the one or more bacterial isolates) can vary; for example the duration can be at least 1 hour, at least 2 hours, at least 4 hours, at least 6 hours, at least 8 hours, at least 10 hours, at least 12 hours, at least 14 hours, at least 16 hours, at least 18 hours, at least 20 hours, at least 22 hours, at least 24 hours, at least 26 hours, at least 28 hours, at least 30 hours, at least 32 hours, at least 34 hours, at least 36 hours, at least 38 hours, at least 40 hours, at least 42 hours, at least 44 hours, at least 46 hours, at least 48 hours, at least 50 hours, at least 52 hours, at least 54 hours, at least 56 hours, at least 58 hours, at least 60 hours, at least 62 hours, at least 64 hours, at least 66 hours, at least 68 hours, at least 70 hours, at least 72 hours, or greater than 72 hours.

In an aspect, a donor can ingest a single or multiple doses of a bacterial isolate to facilitate the incorporation of the bacterial isolate into a fecal microbiota of the donor as a bacterial species. In one aspect, a dose of a bacterial isolate can be ingested by the donor at least once or twice daily for at least three consecutive days or weeks. In another aspect, a dose is ingested at least once, twice, or three times daily for a period between 1 and 16 weeks, between 2 and 16 weeks, between 3 and 16 weeks, between 4 and 16 weeks, between 5 and 16 weeks, between 6 and 16 weeks, between 7 and 16 weeks, between 8 and 16 weeks, between 10 and 16 weeks, between 12 and 16 weeks, between 1 and 12 weeks, between 2 and 12 weeks, between 3 and 12 weeks, between 4 and 12 weeks, between 5 and 12 weeks, between 6 and 12 weeks, between 7 and 12 weeks, between 8 and 12 weeks, between 9 and 12 weeks, between 10 and 12 weeks, between 1 and 2 weeks, between 2 and 3 weeks, between 3 and 4 weeks, between 4 and 5 weeks, between 5 and 6 weeks, between 6 and 7 weeks, between 7 and 8 weeks, between 8 and 9 weeks, between 9 and 10 weeks, or between 10 and 11 weeks.

Further provided herein are kits comprising any herein-disclosed pharmaceutical composition and instructions for use. For example, a kit can include one or more unit dosage forms comprising one or more bacterial mixtures. Such a kit could include for example one or more pharmaceutical compositions comprising a bacterial mixture (e.g., comprising a preparation of fecal bacteria), and optionally a delivery device to administer the composition to the subject, and instructions for administering the dosage to a subject via an appropriate delivery route. In some cases, the dosage form comprises any suitable form of live bacteria (fresh, frozen, lyophilized, etc.) and is formulated for administration to a human subject orally, by nasogastric tube, by colonoscopy, or anally. As described herein, dosage forms suitable for kits provided herein include, without limitation, liquid solutions, capsules, tablets, powders, granules, and lyophilized forms.

The instructions of a kit can describe, for example, dosing information of the one or more pharmaceutical compositions in the kit. As examples, the frequency of administration and dose of a composition, e.g., the number of capsules of a pharmaceutical composition to be administered at a given time, and the number of times of administration per day/week). In an aspect in which the kit comprises more than one composition (e.g., multiple bacterial mixtures or an additional pharmaceutical agent lacking a bacterial mixture), the instructions can describe the dosing of each composition. For example, one composition can be administered before another composition, e.g., sequential administration of the two pharmaceutical compositions separated by minutes, hours, days, weeks, months, or longer. Alternately, two compositions can be administered simultaneously.

It will be appreciated that compositions, dosage forms, and medicaments as described herein include combination pharmaceutical compositions in which one or more additional compounds or medications are added to or otherwise co-administered with a purified fecal microbiota composition.

In an aspect, the present disclosure provides for the following non-limiting embodiments:

1. A method of selecting a subject in need of supplementation with bile acid transforming bacteria that produce a secondary bile acid from a primary bile acid, the method comprising:
determining a number of bacterial clades or species in a microbiome of an individual that comprise a nucleic acid comprising a bile acid metabolism consensus sequence associated with production of the secondary bile acid from the primary bile acid; and
selecting the individual as the subject on the basis of determining that the number of bacterial clades or species is less than a threshold value.
   2. The method of embodiment 1, wherein the bile acid metabolism consensus sequence is at least 20 bp in length and includes all or a portion of a gene encoding a gene product that acts in a metabolic pathway that produces the secondary bile acid.
   3. The method of embodiment 2, wherein the gene is selected from the group consisting of a bai gene, a gene encoding 3α-hydroxysteroid dehydrogenase, a gene encoding 3β-hydroxysteroid dehydrogenase, a gene encoding 5β-reductase, a gene encoding 5α-reductase, a gene encoding 3β-HSDH, and a combination thereof.
   4. The method of any one of embodiments 1 to 3, further comprising the step of administering to the subject the bile acid transforming bacteria.
   5. The method of any one of embodiments 1 to 4, wherein the threshold value is less than five.
   6. The method of any one of embodiments 1 to 5, wherein the threshold value is one.
   7. A method of treating a deficiency in the metabolism of a primary bile acid to a secondary bile acid in a subject in need thereof, the method comprising:
administering a bacterial isolate to the subject to treat the deficiency, wherein the bacterial isolate comprises a bai gene.
   8. The method of embodiment 7, further comprising selecting the subject on the basis of the deficiency.
   9. The method of embodiment 8, wherein said selecting comprises determining from a functional assay that a level of the secondary bile acid is less than a threshold value.
   10. The method of embodiment 9, wherein the functional assay comprises exposing a consortium of fecal bacteria to a primary bile acid substrate and determining the ratio of secondary to primary bile acids.
   11. The method of embodiment 8, wherein said selecting comprises determining that a level of the secondary bile acid in a stool of the subject is less than a threshold value.
   12. The method of embodiment 8, wherein said selecting comprises determining that a level of the primary bile acid in a stool of the subject is greater than a threshold value.
   13. The method of embodiment 8, wherein said selecting comprises determining that no bacteria in a microbiome of the subject comprise a bai operon.
   14. The method of embodiment 8, wherein said selecting comprises determining that the number of bacterial clades or species in a microbiome of the subject comprising a bai operon is less than five.
   15. The method of any one of embodiments 7 to 14, wherein the administered bacterial isolate comprises a bai operon.
   16. The method of any one of embodiments 7 to 15, further comprising administering to the subject a consortium of fecal bacteria from a human donor.
   17. The method of embodiment 16, wherein the consortium of fecal bacteria comprises uncultured fecal bacteria.
   18. The method of embodiment 16, wherein the consortium of fecal bacteria comprises fermented fecal bacteria.
   19. The method of any one of embodiments 7 to 18, wherein the deficiency in metabolism of the primary bile acid to a secondary bile acid is associated with a disorder in the subject.
   20. The method of any one of embodiments 7 to 19, wherein the disorder is selected from the group consisting of inflammatory bowel disease, ulcerative colitis, Crohn's disease, hepatic encephalopathy, hepatitis B, an autism spectrum disorder and a C. difficile infection.
   21. The method of embodiment 16, wherein the bacterial isolate and the consortium of fecal bacteria from a human donor are administered simultaneously.
   22. A method of manufacturing a pharmaceutical composition for administration to a subject to alleviate a deficiency in bile acid metabolism, the method comprising:
selecting a bacterial isolate from a stool of a human donor on the basis of a sequence identity of a nucleic acid of the bacterial isolate to a nucleic acid of a bacterial clade or species in a fecal microbiota of a subject who does not have a history of or is in remission from a disorder related to an intestinal dysbiosis, wherein the nucleic acid comprises a bile acid metabolism consensus sequence that codes for all or a portion of an enzyme that functions in a metabolic pathway producing a primary or a secondary bile acid, and wherein the sequence identity is at least 97%; and
incorporating the bacterial isolate into the pharmaceutical composition.
   23. The method of embodiment 22, wherein the bile acid metabolism consensus sequence comprises a bai gene.
   24. The method of embodiment 22, wherein the subject is in remission as a result of being administered a fecal microbiota transplant (FMT).
   25. The method of embodiment 24, wherein the bacterial clade or species was either not present in the subject prior to the FMT, or was present at a reduced relative abundance in the subject prior to the FMT.
   26. The method of any one of embodiments 22 to 25, wherein the disorder is selected from the group consisting of inflammatory bowel disease, ulcerative colitis, Crohn's disease, hepatic encephalopathy, hepatitis B, an autism spectrum disorder and a C. difficile infection.
   27. The method of any one of embodiments 22 to 25, wherein the sequence identity is at least 99%.
   28. A method, comprising:
selecting a stool of a donor on the basis of the presence of one or more bile acid metabolizing bacteria in the donor, wherein the one or more bile acid metabolizing bacteria are capable of producing a bile acid by transforming a primary bile acid to a secondary bile acid or a first secondary bile acid to a second secondary bile acid;
isolating the one or more bile acid metabolizing bacteria; and
incorporating the isolated bile acid metabolizing bacteria into a pharmaceutical composition.
   29. The method of embodiment 28, wherein the bile acid metabolizing bacteria are capable of transforming a primary bile acid to a secondary bile acid, and the primary bile acid is selected from the group consisting of cholic acid, chenodeoxycholic acid and a combination thereof.
   30. The method of embodiment 28, wherein the bile acid metabolizing bacteria are capable of converting a first secondary bile acid to a second secondary bile acid, and the secondary bile acid is selected from the group consisting of deoxycholic acid (DCA), isodeoxycholic acid, glycodeoxycholic acid (GDCA), taurodeoxycholic acid (TDCA), glycolithocholic acid (GLCA), taurolithocholic acid (TLCA), lithocholic acid (LCA), ursodeoxycholic acid (UDCA), isoursodeoxycholic acid, glycoursodeoxycholic acid (GUDCA), tauroursodeoxycholic acid (TUDCA) or a combination thereof.
   31. The method of any one of embodiments 28 to 30, wherein the one or more bile acid metabolizing bacteria are determined to be present in the donor if a secondary bile acid in stool of the donor is above a threshold level.
   32. The method of any one of embodiments 28 to 30, wherein the one or more bile acid metabolizing bacteria are determined to be present in the donor if a secondary bile acid is produced in a functional assay above a threshold level, wherein the functional assay exposes fecal bacteria of stool of the donor to a bile acid substrate.
   33. The method of embodiment 32, wherein the bile acid substrate is a primary bile acid.
   34. The method of embodiment 33, wherein the bile acid substrate is a secondary bile acid.
   35. The method of embodiment 28, wherein the one or more bile acid metabolizing bacteria are determined to be present in the donor if stool of the donor comprises fecal bacteria having a bile acid metabolism consensus sequence comprising all or a portion of a sequence encoding an enzyme functioning in a metabolic pathway that produces the bile acid.
   36. The method of embodiment 35, wherein the bile acid metabolism consensus sequence comprises a bai gene.
   37. The method of any one of embodiments 28 to 36, wherein the one or more bile acid transforming bacteria are incorporated into the pharmaceutical composition as a consortium of fecal bacteria comprising multiple species of bacteria derived from a single human donor.
   38. A method of treating inflammatory bowel disease in a subject in need thereof, the method comprising:
determining that a fecal microbiota of the subject comprises one or fewer bacterial clades or species capable of transforming a primary bile acid to a secondary bile acid; and
administering to the subject a bacterial species comprising one or more bai genes.
   39. A method of treating a secondary bile acid deficiency in a subject comprising a microbiota lacking a functional bai operon, the method comprising administering to the subject a bacterial species comprising the functional bai operon.
   40. The method of embodiment 39, wherein the subject comprises an intestinal microbiota that lacks the functional bai operon.
   41. A method of restoring a bai operon to a microbiome of a subject lacking a functional copy of the bai gene operon, the method comprising administering to the subject a bacterial isolate comprising the bai gene operon.
   42. The method of any one of embodiments 39 to 41, wherein the bai operon converts CDCA to LCA or CA to DCA.
   43. The method of embodiment 39, wherein said species comprises one or more, two or more, three or more, four or more, five or more, ten or more, fifteen or more, twenty or more strains that comprise the bai operon.
   44. The method of embodiment 39, wherein said species is administered to the subject in the form of a bacterial isolate.
   45. The method of embodiment 39, wherein said species is administered to the subject in the form of multiple bacterial isolates.
   46. A method of treating a disorder related to an intestinal dysbiosis in a subject in need thereof, the method comprising:
determining that a fecal microbiota of the subject comprises three or fewer bacterial clades or species comprising a bile acid metabolism consensus sequence, wherein a bacterial clade or species comprising the bile acid metabolism consensus sequence produces a gene product that catalyzes a step in a metabolic pathway that converts a first secondary bile acid to a second secondary bile acid; and
administering to the subject one or more bile acid metabolizing bacteria comprising a gene encoding the gene product.
   47. A method of treating a subject in need of supplementation with bile acid converting bacteria, the method comprising:
determining a number of bacterial clades or species in a microbiome of an individual that comprise a nucleic acid comprising a bile acid metabolism consensus sequence;
selecting the individual as the subject on the basis of determining that the number of bacterial clades or species is less than a threshold value; and
administering to the subject the bile acid converting bacteria;
wherein the bile acid converting bacteria comprise bacteria that produce isoalloLCA.
   48. A method of treating a subject in need of supplementation with bile acid converting bacteria, the method comprising:
determining a deficiency of a secondary bile acid in the subject,
wherein the deficient secondary bile acid comprises at least isoalloLCA; and
administering to the subject bile acid converting bacteria, wherein following administration the bile acid converting bacteria produce the isoalloLCA in the subject.
   49. The method of embodiment 48, wherein the determining is based on a functional assay that determines a presence or level of a secondary bile acid.
   50. The method of embodiment 48, wherein the determining is based on a functional assay that determines a presence or level of a bile acid converting bacteria.
   51. The method of embodiment 49 or 50, wherein the functional assay is a TGR5 reporter cell assay.
   52. The method of any one of embodiments 47 to 51, wherein the bile acid converting bacteria comprise bacteria from one or more genus selected from the group consisting of Bacteroides, Parabacteroides, Butyricimonas, Odoribacter, Porphyromonas, Alistipes, and a combination thereof.
   53. The method of any one of embodiments 47 to 52, wherein the bile acid converting bacteria comprise bacteria from one or more species selected from the group consisting of Bacteroides uniformis, Bacteroides finegoldii, Bacteroides fragilis, Bacteroides dorei, Bacteroides vulgatus, Bacteroides caccae, Bacteroides ovatus. Bacteroides eggerthii, Bacteroides thetaiotaomicron, Parabacteroides goldsteinii, Parabacteroides distasonis, Parabacteroides merdae, Butyricimonas synergistica, Odoribacter laneus, Porphyromonas somerae, Alistipes indistinctus, Alistipes onderdonkii, Alistipes finegoldii, and a combination thereof.
   54. A method of manufacturing a pharmaceutical composition for administration to a subject to alleviate a deficiency in bile acid metabolism, the method comprising:
selecting a donor based on a presence of isoalloLCA or abundance of isoalloLCA above a threshold level in a stool of the donor;
obtaining at least one bacterial isolate from the stool of the donor, wherein the bacterial isolate comprises a DNA sequence coding for an enzyme that synthesizes isoalloLCA; and
incorporating the at least one bacterial isolate into the pharmaceutical composition.
   55. The method of embodiment 54 wherein the presence or abundance of isoalloLCA is determined based on a TGR5 reporter cell assay.
   56. The method of embodiment 54 or embodiment 55, wherein the bacterial isolate is from one or more genera selected from the group consisting of Bacteroides, Parabacteroides, Butyricimonas, Odoribacter, Porphyromonas, Alistipes, and a combination thereof.
   57. A method comprising:
selecting a stool on the basis of the presence of one or more bile acid metabolizing bacteria in a stool of a donor, wherein the one or more bile acid metabolizing bacteria are capable of producing isoalloLCA;
isolating the one or more bile acid metabolizing bacteria; and
incorporating the bile acid metabolizing bacteria into a pharmaceutical composition.
   58. The method of embodiment 57, wherein the presence of one or more bile acid metabolizing bacteria in a stool of a donor is determined based on a TGR5 reporter cell assay.
   59. The method of embodiment 57, wherein the bile acid metabolizing bacteria is from one or more genera selected from the group consisting of Bacteroides, Parabacteroides, Butyricimonas, Odoribacter, Porphyromonas, Alistipes, and a combination thereof.
   60. A method of treating dysbiosis in a subject in need thereof, the method comprising: determining that a fecal microbiota of the subject comprises one or fewer bacterial clades or species capable of producing isoalloLCA; and
administering to the subject at least one bacterial species comprising a gene selected from the group consisting of a gene encoding 5β-reductase, a gene encoding 5α-reductase, a gene encoding 3β-HSDH, and a combination thereof.
   61. The method of any of embodiments 1 to 21, wherein a TGR5-based cell assay is used to (i) determine a bile acid level, (ii) determine the abundance of the bile acid-transforming bacteria, and/or (iii) select for a bile acid-transforming bacterium.
   62. A method of supplementing a microbiome of a subject with isoalloLCA, the method comprising administering to the subject a DNA sequence that encodes an enzyme that functions in the metabolic pathway that synthesizes isoalloLCA.
   63. The method of embodiment 62, wherein the enzyme is selected from the group consisting of 5β-reductase, 5α-reductase, 3β-HSDH, and a combination thereof.

### EXAMPLES

### Example 1: Subpopulations of Crohn's and UC Patients are Deficient in Secondary Bile Acids

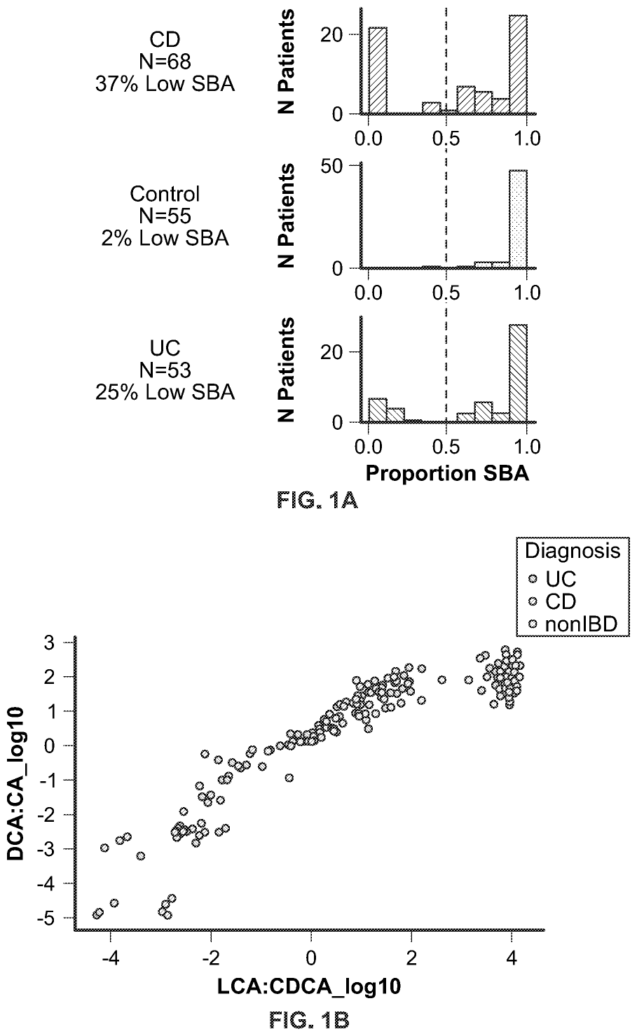

Metagenomic sequencing and untargeted metabolomics profiling data is downloaded from public archives Franzosa, E. A., et al., Gut microbiome structure and metabolic activity in inflammatory bowel disease, Nat Microbiol. 4(2): 293-305 (2018) (“Franzosa 2018”) and processed. The cohort includes 176 subjects: 68 with Crohn's disease (CD), 53 with ulcerative colitis (UC), and 55 non-CD and non-UC controls. FIG. 1A shows that non-CD and non-UC controls have only 2% of the cohort showing low secondary bile acid expression. Of the CD and UC cohorts, 37% and 25% of subjects show low secondary bile acid expression, respectively.

FIG. 1B shows that the ratio of the secondary bile acid DCA to its corresponding primary bile acid CA (DCA:CA_log 10) and the ratio of the secondary bile acid LCA to its corresponding primary bile acid CDCA (LCA:CDCA_log 10) are lower in patients with ulcerative colitis and Crohn's disease relative to non-IBD subjects.

### Example 2: Bile Acid Profiles can be Predicted from Microbial Gene Content

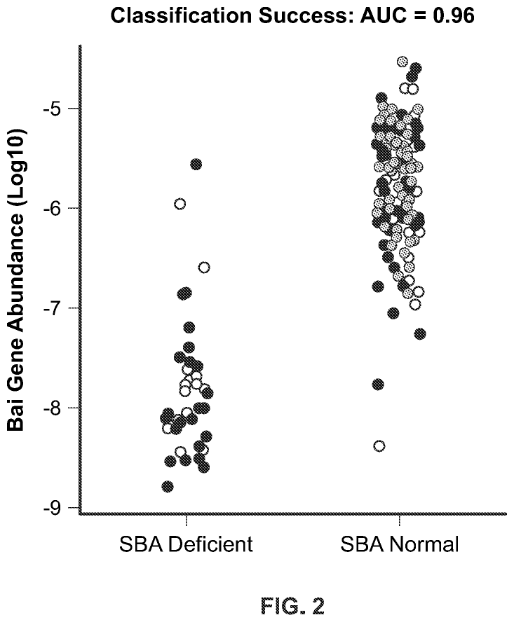

Metagenomic sequencing and untargeted metabolomics profiling data is downloaded from public archives, including Franzosa 2018 and processed. The metabolomic phenotype to distinguish secondary bile acid (SBA) deficient from SBA normal patients is defined as the ratio of the secondary bile acid deoxycholic acid (DCA) to the primary bile acid cholic acid (CA)(“SBA Deficient” corresponds to a DCA:CA ratio of less than 1; “SBA Normal” corresponds to a DCA:CA ratio of 1 or more). To predict this metabolomic phenotype, bile acid-inducible operon (bai) gene abundance is measured from metagenomic data using custom software. FIG. 2 shows a correlation between secondary bile acid expression and bai gene abundance. In the cohort of subjects, patients with normal secondary bile acid levels correlates with increased bai gene abundance.

### Example 3: Presence of Particular Strain in FMT Predicts Crohn's Patient Response to FMT

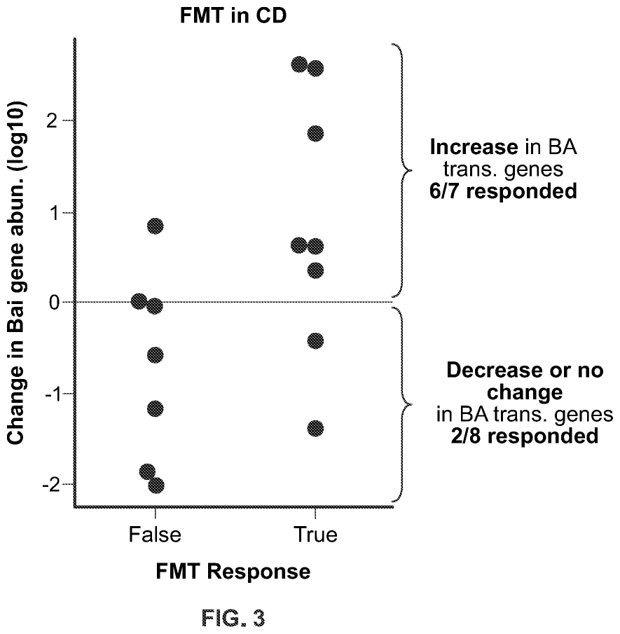

The supplementary figures of Vaughn, B. P, el al., Increased Intestinal Microbial Diversity Following Fecal Microbiota Transplant for Active Crohn's Disease, Inflamm Bowel Dis 22: 2182-2190 (2016), are reviewed to determine the response to FMT. FIG. 3 shows that the repletion of bile acid transforming bacteria predicts a subject's response to FMT. Six of eight subjects show increased bai gene abundance in response to FMT treatment. Whereas of 2 of 8 subjects show a decrease or no change in Bai gene abundance.

### Example 4: FMT Restores the Ability of Microbiome to Produce Secondary Bile Acids

CDI patients are treated with FMT. The number of bile acid conversion genes are detected in the patients prior to FMT treatment and during treatment using shotgun metagenomic data. Specifically, high-throughput sequencing is used to quantify bile acid metabolism genes and pathways (e.g., bile-acid inducible operon (bai) genes required for primary-to-secondary bile acid conversion; FIG. 4A as provided by Ridlon, J. M., et al., “Isolation and Characterization of a Bile Acid Inducible 7α-dehydroxylating operon in Clostridium hylemonae TN271” Anaerobe. 16(2): 137-146 (2010)). FIG. 4B shows that on average and after a single FMT treatment, the number of bai genes detected increase from 3 to 8. Accordingly, FMT treatment restores the genetic operon required for the production of secondary bile acids.

### Example 5: Shotgun Metagenomics to Determine the Abundance of Bile-Acid Transforming Genes in a Stool Sample

Reference sequences for the bai family of enzymes from Vital, M., et al., “Diversity of Bacteria Exhibiting Bile Acid-inducible 7α-dehydroxylation Genes in the Human Gut” Comput Struct Biotechnol. J. 17:1016-1019 (2019) (“Vital 2019”) are downloaded. Each of the genome's set of 8 bai genes are clustered at the 90% amino acid sequence level. The raw shotgun reads for each sample are mapped to a representative set of bai reference sequences representing each cluster, and each read is assigned to its best hit. From the mapping results the overall abundance of bai genes in each sample is calculated across all 8 bai genes of the proportion of reads that map to that gene category. This is repeated within each 90% bai cluster to estimate the abundance of each cluster. The proportion of bai genes out of a total of 8 possible genes are also detected in each sample, and the proportion of bai genes detected from each cluster.

As shown in FIGS. 5A and 5B, the detection of a single bile acid transforming clade can be a predictor of high secondary bile acids. In FIG. 5A, “SBA Deficient” and “SBA Normal” are defined as a ratio of DCA:CA of less than 1, and 1 or more, respectively. In FIG. 5B, “low_SBA” and “high_SBA” are defined as a ratio of LCA:CDCA of less than 1, and 1 or more, respectively. In secondary bile acid deficient samples the number of bile acid transforming clades are low relative to samples with normal levels of bile acids. For FIG. 5A, only one subject having a ratio of DCA:CA of less than 1 has 2 clades with the bai gene operon, whereas 9 subjects have 1 clade, and 25 subjects have 0 clades. For FIG. 5B, 6 subjects having a ratio of LCA:CDCSA have 2 clades, 11 subjects have 1 clade and 26 samples show 0 clades. This contrasts with subjects having a ratio of secondary bile acid:primary bile acid which is normalized (greater than 1). In both FIG. 5A and FIG. 5B, all such subjects have at least one clade of bacteria with the bai gene operon, and the great majority (more than 120 for each of FIGS. 5A and 5B) have 2 or more. This figure includes patients with CD, UC and non-IBD controls.

### Example 6: 16S Sequencing to Determine the Abundance of Bile-Acid Transforming Genes in a Stool Sample

Reference sequences for the bai family of enzymes from Vital 2019 are downloaded. Each of the genome's set of 8 bai genes are clustered at the 90% amino acid sequence level. The previously published genomes from various sources used by the Vital paper are found and the 16S sequence is extracted from each genome that has bai genes. 16S reads are mapped to the set of 16S reference sequences from bai genomes. The overall abundance of taxa with bai genes and the proportion of 16S reads that map to any of the reference sequences are calculated. The abundance of each bai cluster compared to the proportion of 16S reads that map to reference sequences from genomes in that cluster are also calculated.

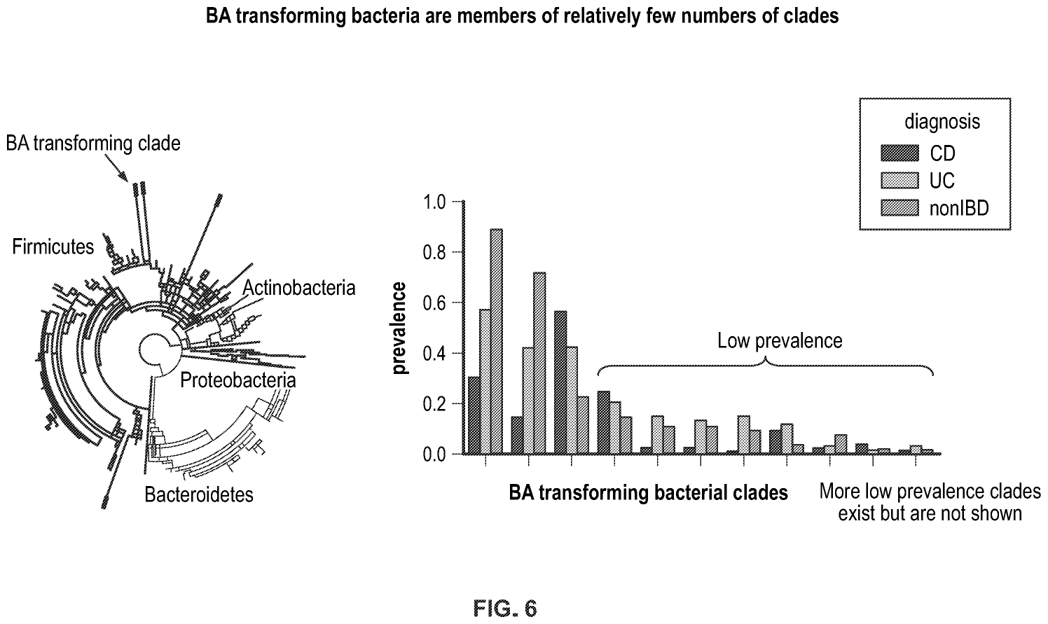

As shown in FIG. 6A bile acid transforming bacteria are members of relatively few numbers of clades in Firmicutes phylum. Also, the prevalence of bile acid transforming clades vary between stool from normal IBD donors and Crohn's Disease and ulcerative colitis diagnosed donors (FIG. 6B).

### Example 7: Depletion of Bile-Acid Transforming Bacteria Prevents Downstream Metabolism of Secondary Bile-Acids into an Array of Beneficial Compounds

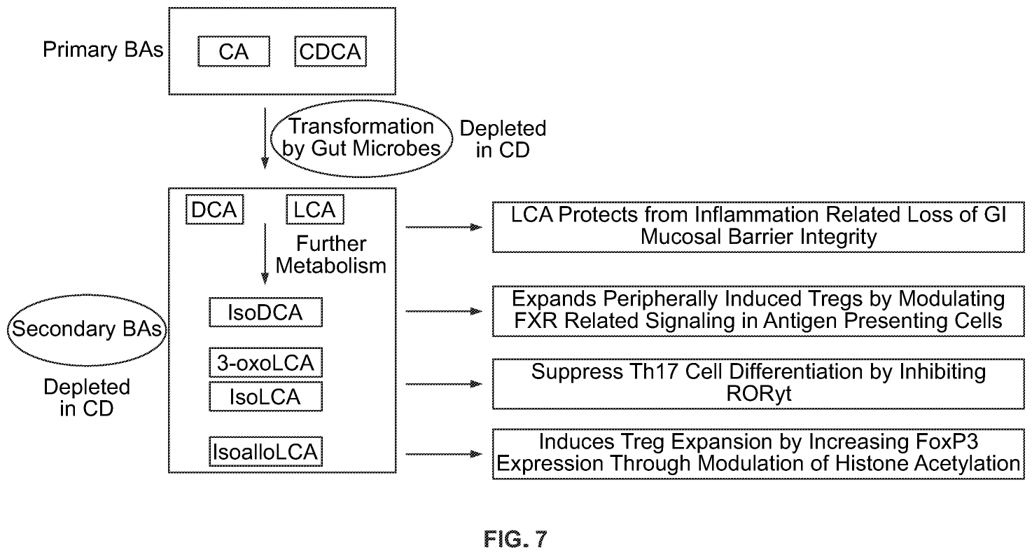

Primary bile-acids (CA and CDCA) are normally transformed by gut microbes to secondary bile-acids (DCA and LCA). The secondary bile-acids are further metabolized to isodeoxycholic acid (IsoDCA), dehydrolithocholic acid (3-oxoLCA), isolithocholic acid (isoLCA), and IsoalloLCA. LCA protects from inflammation related loss of gastrointestinal mucosal barrier integrity. IsoDCA expands peripherally induced regulatory T cells (Tregs) by modulating farnesoid X receptor (FXR) related signaling in antigen presenting cells. 3-oxoLCA and IsoLCA suppress Th17 cell differentiation by inhibiting RAR-related orphan receptor gamma (RORyt). IsoalloLCA induces Treg expansion by increasing Forkhead Box P3 (FoxP3) expression through modulation of histone acetylation. As shown in FIG. 7, the transformation of primary bile-acids to secondary bile-acids and further metabolism is depleted in Crohn's disease patients.

### Example 8: Crohn's Disease Patients Depleted of Bile-Acid Transforming Bacteria Lack Secondary Bile-Acids

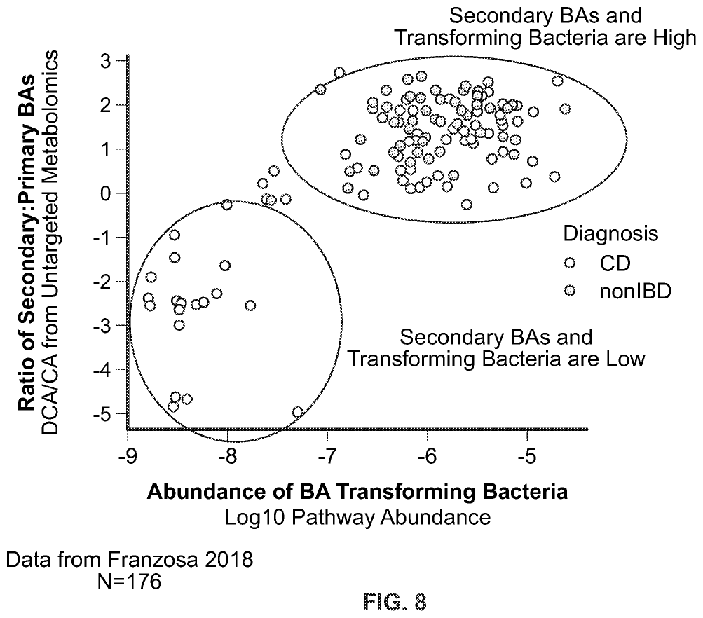

Metagenomic sequencing and untargeted metabolomics profiling data is downloaded from public archives Franzosa, E. A., et al., Gut microbiome structure and metabolic activity in inflammatory bowel disease, Nat Microbiol. 4(2): 293-305 (2018) (“Franzosa 2018”) and processed. The cohort includes 176 subjects: 68 with Crohn's disease (CD), 53 with ulcerative colitis (UC), and 55 non-CD and non-UC controls. FIG. 8 shows that non-CD and non-UC controls (collectively non-IBD) shows an increased ratio of secondary bile-acid and primary bile acids correlates with an increased abundance of bile-acid transforming bacteria, compared to Crohn's disease patients.

### Example 9: Thin Laver Chromatography Pilot Experiment

A limit of detection experiment is performed to determine the concentrations of bile acids cholic acid (CA), deoxygcholic acid (DCA), and lithocholic acid (LCA) that are detected by Thin Layer Chromatograph (TLC) in order to develop a screen of bile acid transformation during development and actual isolation experiments. The protocol is adapted from Doden et. al Applied and Env. Micro (2018). The utility of two visualization methods, phosphomolybdic acid (PMA) and sulfuric acid, is determined by preparing two plates with the same samples that are each sprayed with a different visualization agent. Visualization methods can be found in Pyka, A. J of Liquid Chromatography & Related Technologies (2008).

Materials include the following:

Bile acid stocks;

### Methanol;

hazard statements:
   highly flammable liquid and vapor
   toxic if swallowed
   toxic if in contact with skin
   toxic if inhaled
   causes damage to organs
      Microcentrifuge tubes;

### Cyclohexane;

hazard statements:
   highly flammable liquid and vapor
   causes skin irritation
   may cause drowsiness or dizziness
   may be fatal if swallowed and enters airways
   very toxic to aquatic life with long lasting effects
      Ethyl acetate;
hazard statements:
   highly flammable liquid and vapor
   causes skin irritation
   may cause drowsiness or dizziness
      Glacial acetic acid;
hazard statements:
   Corrosive, flammable liquid and vapor.
   Causes severe digestive and respiratory tract burns.
   Causes severe eye and skin burns.
   May be harmful if absorbed through the skin.
Acetic acid forms icelike solid below 17° C. (62° F.).
   TLC plates;

### Pencil;

### Ruler;

Capillary spotting tubes;
Phosphomolybdic acid;
hazard statements:
   Highly flammable liquid and vapor.
   May intensify fire; oxidizer.
   Causes severe skin burns and eye damage.
   May cause drowsiness or dizziness.
      Sulfuric acid;
hazard statements:
   may be corrosive to metals
   causes severe skin burns and eye damage
   causes serious eye damage
      Lidded glass containers;

### Forceps; and

Spray bottles.

First, 50 mM stocks of cholic acid (CA), chenodeoxycholic acid (CDCA), deoxycholic acid (DCA), and lithocholic acid (LCA) are prepared in methanol. The 50 mM stocks are diluted in methanol to 250 micromolar (μM) in a fume hood. The 250 μM stocks are then diluted 1:5 to prepared additional standards by adding methanol. The standards are 50 μM, 10 μM, and 2 μM.

Next, the hood service is covered with diaper paper. Two glass containers with lids are used to prepare 50 ml of solvent by adding 24 ml cyclohexane, 24 ml ethyl acetate, and 2 ml glacial acetic acid. Both visualization stains, phosphomolybdic acid (PMA) and sulfuric acid, are prepared. Phosphomolybdic acid (10 grams) is dissolved in 100 mL of 100% ethanol for PMA. Concentrated sulfuric acid (5 ml) is added to 95 ml of methanol.

The heat gun stand with a ring clamp is set up in the fume hood that has an accessible electrical outlet. The ring clamp is used to secure the head gun. The heat gun is positioned 10 cm from the TLC plate that is heated.

TLC plates (20×20 cm) are prepared by marking a horizontal line as the origin about 1 cm from the bottom of the plate and another horizontal line 25 mm from the top of the plate (to indicate where the solvent front travels to) with a pencil. Caution is taken to not touch the silica gel by using forceps to hold the plate in place. Twenty lanes are numbered along the origin line of the plate and spaced 1 cm apart. Using a clean capillary spotting tube each time, each BA stock is spotted on the origin line of both plates in the lane designated in a corresponding table and each spot is allowed to dry. Careful application is used so that capillary tubes only briefly touch the plate and the method is consistent between samples, spotting a similar amount of each sample. Once the spots have dried, the TLC plate is placed a glass container, covered tightly with a lid, and allowed to develop. The origin line of each spot is above the solvent and not submerged. The plate is removed from the glass container using forceps when the solvent reaches approximately 25 mm from the top of the plate. The plate is marked to shown where the solvent front stopped before it dries. The plate is sprayed with the PMA visualization solution. The plate is heated to 120° C. for 20 mins using the heat gun at the correct distance as provided above. After heating, visible spots in each lane are circled and the distance traveled from the origin is measured using a ruler.

A second plate is prepared as provided above. However, the second plate is sprayed with sulfuric acid visualization solution instead of PMA. The plate is heated to 90° C. for 20 min using the heat gun at the correct distance, as provided above. After heating, visible spots in each lane are circled and the distance traveled from the origin is measured using a ruler.

### Example 10: Selection of Bile Acids with Consensus Sequence

Donor stool is analyzed to determine the presence of bai proteins encoded by bacteria in donor stool. Examples of bai proteins that are encoded by bacteria added to a composition for treatment are provided in SEQ ID NOs: 1-252.

### Example 11: Bile Acid Reporter Assay

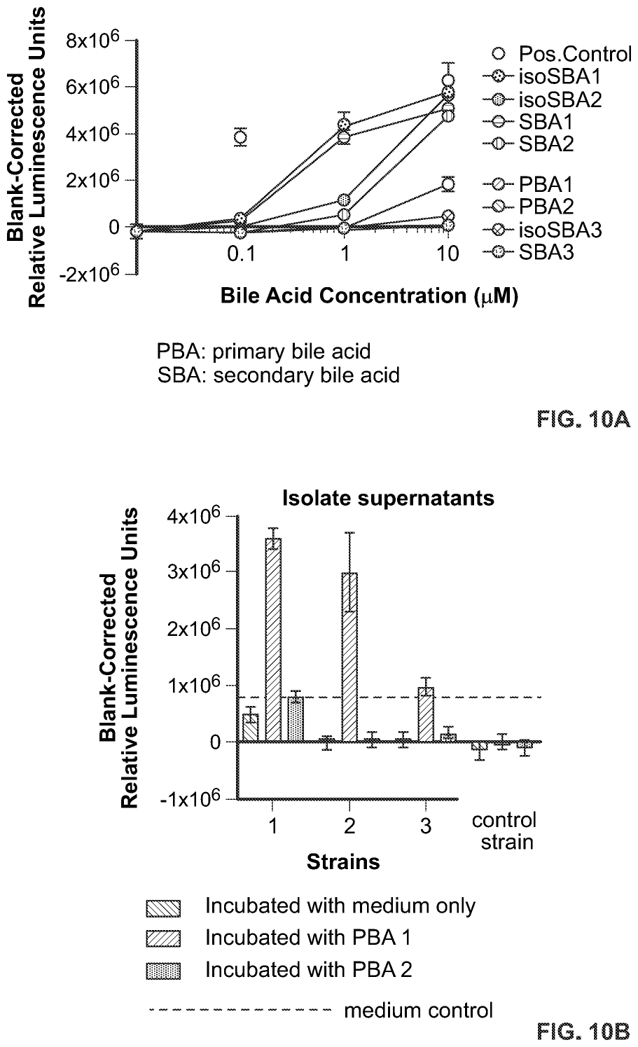

TGR5 is a G-protein-coupled receptor which exhibits a higher affinity for secondary bile acids than for primary bile acids. A protocol is developed for measuring TGR5 receptor activation with diverse complex bacterial media following fermentation of bacterial isolates with input substrates, i.e., various precursor bile acids and appropriate media. The TGR5 reporter cell assay uses a luminescence-based system to read out on the levels of TGR5 activation by stimulations of interest (TGR5 agonists, bile acids, bacterial supernatants, etc.). Using a 96-well-format kit (Cayman Chemicals, Item No. 601440), HEK293 cells are transfected with a complex to express TGR5 on the cell surface and a cyclic AMP response element regulating a secreted heat-resistant alkaline phosphatase reporter. Once expression occurs, HEK293 cells are treated with the stimulants of interest, allowing interaction with the TGR5 on the cell surface. This sets off a signaling cascade resulting in secretion of the heat-resistant alkaline phosphatase into the culture media, which is collected after 6-8 hours and heat-treated to inactivate any endogenous alkaline phosphatase. A substrate is then added to the supernatants collected from the HEK293 cells, resulting in luminescence upon interaction with the secreted heat-resistant alkaline phosphatase, allowing the luminescence of the supernatant to be read using a standard spectrophotometer as a metric of TGR5 activation. In this study bacterial isolates with bile acid transformation activity are incubated with primary bile acids (PBA) to allow for fermentation. Then, the supernatants from the bacterial isolates are collected and tested for the presence of secondary bile acids (SBA) and their ability to activate TGR5 using the TGR5 reporter cell assay. FIG. 10A depicts luminescence readings of cell assays testing medium containing primary bile acids chenodeoxycholic acid (CDCA) and cholic acid (CA) (PBA1 and PBA2 respectively) as controls and medium from bacterial isolate fermentations to secondary bile acids 3-oxo-lithocholic acid (3-oxo-LCA, 30LCA), isolithocholic acid (isoLCA, ILCA), lithocholic acid (LCA), deoxycholic acid (DCA), alloisolithocholic acid (alloisoLCA, AILCA, isoalloLCA, IALCA), and ursodeoxycholic acid (UDCA) (isoSBA1, isoSB2, SBA1, SBA2, isoSBA3, and SBA3 respectively). Additionally, 3-(2-chlorophenyl)-N-(4-chlorophenyl)-N,5-dimethylisoxazole-4-carboxamide was used as a positive control. FIG. 10B depicts luminescence readings from four bacterial isolates (1: Clostridium scindens (strain 1); 2: Clostridium scindens (strain 2); 3: Clostridium hylemonae; control: Bacteroides fragilis) incubated with medium only or with primary bile acids cholic acid (CA) and chenodeoxycholic acid (CDCA) (PBA1 and PBA2 respectively).

### Example 12: isoalloLCA as a Biomarker for Donor Selection and Patient Response

Metagenomic sequencing and untargeted metabolomics profiling data is downloaded from public archives including Franzosa, E. A., et al., Gut microbiome structure and metabolic activity in inflammatory bowel disease, Nat Microbiol. 4(2): 293-305 (2018) (“Franzosa 2018”) and Human Microbiome Project (HMP2), and processed. Data from 220 analyzed samples from the Franzosa 2018 archive is used, which includes data from 56 non-inflammatory bowel disease (IBD) samples, 76 ulcerative colitis (UC) samples, and 88 Crohn's disease (CD) samples. Additionally, metagenomic data from 130 analyzed samples from the HMP2 archive is used, which includes data from 27 non-LBD samples, 38 UC samples, and 65 CD samples.

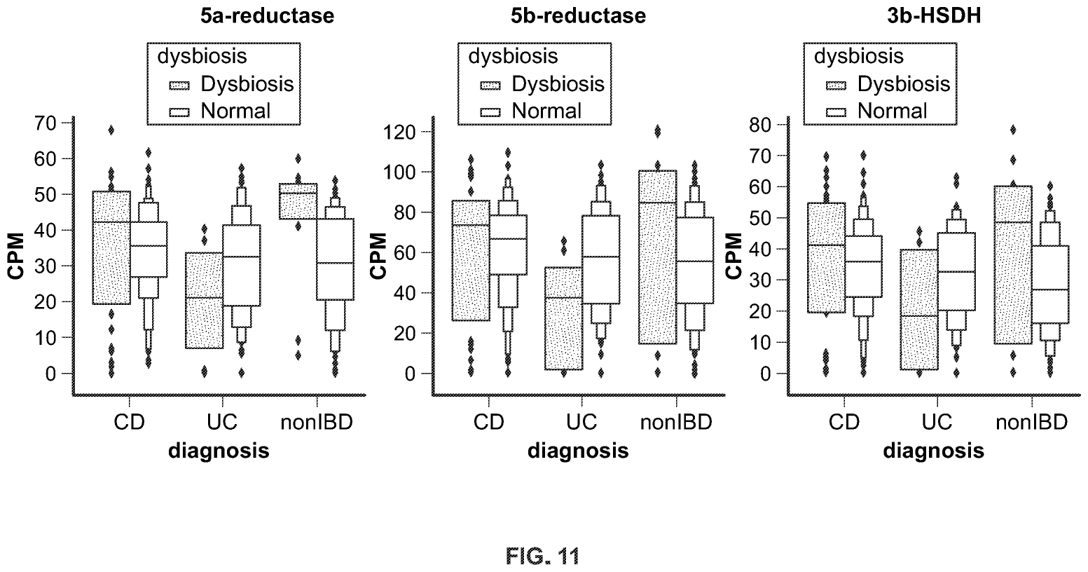

IsoalloLCA is a secondary bile acid which induces Treg expansion by increasing Forkhead Box P3 (FoxP3) expression through modulation of histone acetylation. Samples from subjects with CD and UC show secondary bile acid deficiency. At least 18 distinct isoalloLCA-producing bacterial species exist. See Li, Wei et al. “A bacterial bile acid metabolite modulates Treg activity through the nuclear hormone receptor NR4A1.” Cell host & microbe vol. 29, 9 (2021): 1366-1377; Sato, Yuko et al. “Novel bile acid biosynthetic pathways are enriched in the microbiome of centenarians.” Nature vol. 599, 7885 (2021): 458-464. The metagenomic data is analyzed for isoalloLCA sequences, and the abundance of isoalloLCA-producing bacterial species in non-IBD, UC, and CD samples is determined (data not shown). Additionally, an analysis of the abundance of characterized isoalloLCA-producing genes (5α-reductase, 5β-reductase, and 3β-HSDH) in non-IBD, UC, and CD samples where patients had been further classified as dysbiotic or normal, reveals that isoalloLCA-producing genes are differentially present in the microbiome of dysbiotic versus normal states regardless of the non-IBD, UC, or CD diagnosis (FIG. 11). These studies indicate that isoalloLCA can be used as a marker of dysbiosis, allowing for the selection of a subject in need of supplementation with bile acid transforming bacteria, particularly isoalloLCA-producing bacteria. These studies also indicate that isoalloLCA can be used as a marker to identify suitable stool donors whose microbiomes contain beneficial isoalloLCA-producing bacteria.

A variety of further modifications and improvements in and to the compositions and methods of the present disclosure will be apparent to those skilled in the art.

### Example 13: Bile Acid Stability Studies

Bile acid stability is investigated using clinically relevant raw stool sample collection procedures where stool samples are held for long times during shipment to a central laboratory for intake and freezer storage. Three stool donations are collected, and each is homogenized and split into two subsamples. One subsample is immediately placed in storage at 4° C. The other subsample is spiked with primary bile acids CA and CDCA prior to storage at 4° C. to better assess primary bile acid stability. Samples are collected from each subsample at 0, 6, 24, 48, and 72 hours to determine stability of bile acids during storage. At each time point 0.5 g aliquots are collected and frozen until shipment for quantitative bile acid metabolomics analysis. Large reductions in primary bile acid concentrations are observed with longer storage. Smaller relative changes in secondary bile acids are observed, suggesting greater stability of these metabolites. While an increase in secondary bile acids is observed in samples receiving primary bile acid spikes, higher concentration of secondary bile acids compared to primary bile acids in these stool samples minimizes the impact of conversion of primary bile acids on measured secondary bile acid concentrations. This study reveals that primary bile acid stability over timespans that are realistic for clinical sample collection is poor and alternate sampling methods to stabilize primary bile acid concentrations are required. While raw stool collection may still be reliable for revealing large, order of magnitude changes in primary bile acids that are not observed in this study, stabilizing at home collection methods may offer a potential solution to storage of raw stool samples.

1. A method of selecting a subject in need of supplementation with bile acid transforming bacteria that produce a secondary bile acid from a primary bile acid, the method comprising:
determining a number of bacterial clades or species in a microbiome of an individual that comprise a nucleic acid comprising a bile acid metabolism consensus sequence associated with production of the secondary bile acid from the primary bile acid; and
selecting the individual as the subject on the basis of determining that the number of bacterial clades or species is less than five.

2. The method of claim 1, wherein the bile acid metabolism consensus sequence is at least 20 bp in length and includes all or a portion of a gene encoding a gene product that acts in a metabolic pathway that produces the secondary bile acid.

3. The method of claim 2, wherein the gene is selected from the group consisting of a bai gene, a gene encoding 3α-hydroxysteroid dehydrogenase, a gene encoding 3β-hydroxysteroid dehydrogenase, a gene encoding 5β-reductase, a gene encoding 5α-reductase, a gene encoding 3β-HSDH, and a combination thereof.

4. The method of claim 1, further comprising the step of administering to the subject the bile acid transforming bacteria.

5. (canceled)

6. (canceled)

7. A method of treating a deficiency in the metabolism of a primary bile acid to a secondary bile acid in a subject in need thereof, the method comprising:
administering a bacterial isolate to the subject to treat the deficiency, wherein the bacterial isolate comprises a bai gene,
wherein the subject is selected by determining that
(a) a level of the secondary bile acid in a stool of the subject is less than a threshold value, or
(b) a level of the primary bile acid in a stool of the subject is greater than a threshold value.

8.-14. (canceled)

15. The method of claim 7, wherein the administered bacterial isolate comprises a bai operon.

16. The method of claim 7, further comprising administering to the subject a consortium of fecal bacteria from a human donor.

17. The method of claim 16, wherein the consortium of fecal bacteria comprises uncultured fecal bacteria.

18. The method of claim 16, wherein the consortium of fecal bacteria comprises fermented fecal bacteria.

19. (canceled)

20. The method of claim 19, wherein the deficiency in metabolism of the primary bile acid to a secondary bile acid is associated with a disorder is selected from the group consisting of inflammatory bowel disease, ulcerative colitis, Crohn's disease, hepatic encephalopathy, hepatitis B, an autism spectrum disorder and a C. difficile infection.

21.-28. (canceled)

29. The method of claim 1, wherein the bile acid transforming bacteria are capable of transforming a primary bile acid to a secondary bile acid, and (i) the primary bile acid is selected from the group consisting of cholic acid, chenodeoxycholic acid and a combination thereof or (ii) the secondary bile acid is selected from the group consisting of deoxycholic acid (DCA), isodeoxycholic acid, glycodeoxycholic acid (GDCA), taurodeoxycholic acid (TDCA), glycolithocholic acid (GLCA), taurolithocholic acid (TLCA), lithocholic acid (LCA), ursodeoxycholic acid (UDCA), isoursodeoxycholic acid, glycoursodeoxycholic acid (GUDCA), tauroursodeoxycholic acid (TUDCA) or a combination thereof.

30.-36. (canceled)

37. The method of claim 4, wherein the bile acid transforming bacteria are derived from a human donor.

38. The method of claim 4, wherein the bile acid transforming bacteria comprises
one or more bai genes.

39.-47. (canceled)

48. A method of treating a subject in need of supplementation with bile acid converting bacteria, the method comprising:
determining a deficiency of a secondary bile acid in the subject,
wherein the deficient secondary bile acid comprises at least isoalloLCA; and
administering to the subject bile acid converting bacteria, wherein following administration the bile acid converting bacteria produce the isoalloLCA in the subject.

49. The method of claim 48, wherein the determining is based on a functional assay that determines a presence or level of a secondary bile acid or bile acid converting bacteria.

50. (canceled)

51. The method of claim 49, wherein the functional assay is a TGR5 reporter cell assay.

52. The method of claim 48, wherein the bile acid converting bacteria comprise bacteria from one or more genus selected from the group consisting of Bacteroides, Parabacteroides, Butyricimonas, Odoribacter, Porphyromonas, Alistipes, and a combination thereof.

53. The method of claim 48, wherein the bile acid converting bacteria comprise bacteria from one or more species selected from the group consisting of Bacteroides uniformis, Bacteroides finegoldii, Bacteroides fragilis, Bacteroides dorei, Bacteroides vulgatus, Bacteroides caccae, Bacteroides ovatus, Bacteroides eggerthii, Bacteroides thetaiotaomicron, Parabacteroides goldsteinii, Parabacteroides distasonis, Parabacteroides merdae, Butyricimonas synergistica, Odoribacter laneus, Porphyromonas somerae, Alistipes indistinctus, Alistipes onderdonkii, Alistipes finegoldii, and a combination thereof.

54.-61. (canceled)

62. The method of claim 48, further comprising administering to the subject a DNA sequence that encodes an enzyme that functions in the metabolic pathway that synthesizes isoalloLCA.

63. The method of claim 62, wherein the enzyme is selected from the group consisting of 5β-reductase, 5α-reductase, 3β-HSDH, and a combination thereof.
